(19) United States
(12) Patent Application Publication
(10) Pub. No.: US 2026/0207777 A1
(43) Pub. Date: Jul. 23, 2026

(54) IMPROVED PEPTIDE INHIBITORS OF P53 BINDING PROTEIN 53BP1

(71) Applicant: KAMAU THERAPEUTICS, INC., South San Francisco, CA (US)

(72) Inventors: Juan PEREZ-BERMEJO, South San Francisco, CA (US); Oghene EFAGENE, South San Francisco, CA (US); William MATERN, South San Francisco, CA (US); James PARTRIDGE, South San Francisco, CA (US); Kristen SEIM, South San Francisco, CA (US); Vanessa SOROS, South San Francisco, CA (US); Beeke WIENERT, South San Francisco, CA (US)

(21) Appl. No.: 19/140,941

(22) PCT Filed: Dec. 18, 2023
(86) PCT No.: PCT/US2023/084675

§ 371 Date: Jun. 18, 2025

Related U.S. Application Data
(60) Provisional application No. 63/476,120, filed on Dec. 19, 2022.
(60) Provisional application No. 63/490,447, filed on Mar. 15, 2023.

(51) Int. Cl. A61K 48/00; A61K 31/4985; A61K 31/522; A61K 31/5377; C07K 14/47; C12N 9/22; C12N 15/11; C12N 15/86; C12N 15/90; G01N 33/68

(52) U.S. Cl. CPC A61K 48/005; A61K 31/4985; A61K 31/522; A61K 31/5377; A61K 48/0083; C07K 14/4702; C12N 9/226; C12N 15/111; C12N 15/86; C12N 15/907; G01N 33/6893; C12N 2310/20; C12N 2310/315; C12N 2310/321; C12N 2750/14143; G01N 2333/4703

(57) ABSTRACT

Compositions and methods are provided for enhancing homology-directed repair in gene-editing applications using improved inhibitors of 53BP1.

### CROSS REFERENCE TO RELATED APPLICATIONS

This application claims the benefit of, and priority to, U.S. Provisional Application No. 63/476,120, filed Dec. 19, 2022, and U.S. Provisional Application No. 63/490,447, filed Mar. 15, 2023, each of which is hereby incorporated herein by reference in its entirety for all purposes.

### FIELD

Provided herein are methods and compositions for increasing homology-directed repair including homologous recombination using inhibitors of 53BP1.

### BACKGROUND

Gene editing modifies the genomes of individual cells at one or more targeted loci. In some instances of therapeutic gene editing, such modification is made at one or more targeted loci associated with a disease. Given that each human cell has two copies of the genome, gene editing procedures such as those utilizing CRISPR/Cas systems can give rise to various genomic outcomes in a targeted cell population, including cells having no change to either allele of the targeted locus, changes to only one allele, or changes to both alleles. In those alleles bearing changes, modifications can include insertions or deletions (INDELS) of one or more nucleotides, or homology-directed repair (HDR)-mediated insertion of donor polynucleotides that aim to address (e.g. precisely correct) one or more mutation(s) of the targeted locus. Cell populations having undergone the editing procedure can then be administered to a patient, for example, a patient whose hematopoietic stem cells were harvested and edited ex vivo, as an aim to treat or cure the underlying disease.

Non-homologous end-joining (NHEJ) represents the dominant pathway by which DNA double-stranded breaks (DSBs) are repaired in a mammalian cell, and this pathway competes with repair via the homology-directed repair route. Repair of DSBs is regulated by p53-binding protein 1 (53BP1) which promotes NHEJ, and BRCA1 which promotes HR. 53BP1 blocks long-range DNA end resection and inhibits recruitment of BRCA1 to DSBs. 53BP1 also suppresses the formation of 3′ single-stranded DNA tails, the rate-limiting step for the initiation of HR. Inhibition of 53BP1 function can result in increased HR activity, which can improve gene editing efficiency where HR is needed. Thus, there is a need for improved 53BP1 inhibitors to improve the efficiency of HDR-mediated gene editing approaches.

Sickle cell disease (SCD) is a genetic condition caused by a single point mutation at codon 6 in both copies of the beta-globin (HBB) gene, resulting in an E6V mutation that gives rise to sickle(S) hemoglobin (HbS) production instead of adult (A) hemoglobin (HbA). Gene-edited autologous hematopoietic stem cell-based therapies in clinical development for SCD include those that are designed to directly correct the underlying point mutation via HDR-mediated integration of a corrective nucleic acid sequence, thereby decreasing HbS production and restoring HbA expression. Increasing the rate of HDR in such gene-editing methods is desirable to improve the efficiency or producing autologous cell products for the treatment of sickle cell disease (SCD) as well as any other condition to be treated by gene-edited autologous (or even allogenic) cell therapies.

### SUMMARY

The present disclosure provides compositions and methods for enhancing HDR-mediated integration of polynucleotides in gene editing applications. The methods utilize polypeptide compositions that bind to the p53-binding protein 1 (53BP1) and inhibit 53BP1's promotion of the NHEJ DNA repair pathway, in favor of HDR-mediated DNA repair.

Thus, in one aspect, provided herein is a polypeptide comprising an amino acid sequence having at least 60% sequence identity to the amino acid sequence of SEQ ID NO 1, and having one or more modifications relative to SEQ ID NO: 1, wherein the one or more modifications comprise a modification at position 67 of SEQ ID NO: 1, a modification at position 68 of SEQ ID NO: 1, or a combination thereof. In preferred embodiments, the polypeptide is capable of inhibiting the activity of 53BP1. In some embodiments, the polypeptide comprises the amino acid sequence of SEQ ID NO: 1 having one or more modifications relative to SEQ ID NO: 1, wherein the one or more modifications comprise a modification at position 67 of SEQ ID NO: 1, a modification at position 68 of SEQ ID NO: 1, or a combination thereof. In some embodiments, the modification at position 67 of SEQ ID NO: 1 is Arg (L67R), His (L67H), Lys (L67K), Ser (L67S), Thr (L67T), Gln (L67Q), Asn (L67N), or His (L67H). In some embodiments, the modification at position 68 of SEQ ID NO: 1 is Trp (H68W), Tyr (H68Y), or Phe (H68F).

In some embodiments, the polypeptide comprises one or more modifications selected from the group consisting of L67R, L67H, L67K, L67S, L67T, L67Q, L67N, L67H, H68W, H68Y, H68F, and combinations thereof. In some embodiments, the one or more modifications are selected from the group consisting of: (a) an Arg at position 67 of SEQ ID NO: 1 (L67R), and a Trp at position 68 of SEQ ID NO: 1 (H68W); (b) a His at position 67 of SEQ ID NO: 1 (L67H), and a Tyr at position 68 of SEQ ID NO: 1 (H68Y); (c) a His at position 67 of SEQ ID NO: 1 (L67H), and a Phe at position 68 of SEQ ID NO: 1 (H68F); (d) a His at position 67 of SEQ ID NO: 1 (L67H), and a Trp at position 68 of SEQ ID NO: 1 (H68W); (e) an Arg at position 67 of SEQ ID NO: 1 (L67R), and a Tyr at position 68 of SEQ ID NO: 1 (H68Y); (f) an Arg at position 67 of SEQ ID NO: 1 (L67R), and a Phe at position 68 of SEQ ID NO: 1 (H68F); (g) a Ser at position 67 of SEQ ID NO: 1 (L67S), and a Phe at position 68 of SEQ ID NO: 1 (H68F); (h) a Thr at position 67 of SEQ ID NO: 1 (L67T), and a Trp at position 68 of SEQ ID NO: 1 (H68W); (i) a Ser at position 67 of SEQ ID NO: 1 (L67S), and a Trp at position 68 of SEQ ID NO: 1 (H68W); (j) a Gln at position 67 of SEQ ID NO: 1 (L67Q), and a Tyr at position 68 of SEQ ID NO: 1 (H68Y); (k) an Asn at position 67 of SEQ ID NO: 1 (L67N), and a Tyr at position 68 of SEQ ID NO: 1 (H68Y); (1) an Asn at position 67 of SEQ ID NO: 1 (L67N), and a Trp at position 68 of SEQ ID NO: 1 (H68W); and (m) a Lys at position 67 of SEQ ID NO: 1 (L67K), and a Tyr at position 68 of SEQ ID NO: 1 (H68Y).

In some embodiments, the polypeptide comprises the sequence of any one of SEQ ID NOs: 2 to 72. In some embodiments, the polypeptide consists of the sequence of any one of SEQ ID NOs: 2 to 72. In particular embodiments, the polypeptide comprises or consists of the sequence of SEQ ID NO: 2. In other particular embodiments, the polypeptide comprises or consists of the sequence of SEQ ID NO: 3.

In some embodiments, the polypeptide further comprises one or more additional modifications relative to SEQ ID NO: 1. In some embodiments, the polypeptide the one or more additional modifications relative to SEQ ID NO: 1 comprise 1, 2, 3, 4, 5, 6, 7, 8, 9, 10, 11, 12, 13, 14, 15, 16, 17, 18, 19, 20 or more modifications at any amino acid position relative to the amino acid sequence of SEQ ID NO: 1. In some embodiments, the polypeptide the one or more additional modifications are selected from the group consisting of: (a) a Gln at position 2 of SEQ ID NO: 1 (L2Q); (b) an Ile at position 44 of SEQ ID NO: 1 (A44I); (c) a Gln at position 49 of SEQ ID NO: 1 (S49Q); (d) a Gln at position 62 of SEQ ID NO: 1 (L62Q); (e) a Glu at position 64 of SEQ ID NO: 1 (D64E); (f) a Thr at position 66 of SEQ ID NO: 1 (K66T); (g) a Leu at position 69 of SEQ ID NO: 1 (P69L); (h) a Val at position 70 of SEQ ID NO: 1 (L70V); and combinations thereof. In some embodiments, the polypeptide further comprises one or more modifications at: (a) position 12 of SEQ ID NO: 1; (b) position 14 of SEQ ID NO: 1; (c) position 65 of SEQ ID NO 1; (d) position 66 of SEQ ID NO: 1; (e) position 67 of SEQ ID NO:1; and/or (f) combinations thereof. In some embodiments, the modification at position 12 of SEQ ID NO: 1 is Tyr (T12Y). In some embodiments, the modification at position 12 of SEQ ID NO: 1 is Val (T12V). In some embodiments, the modification at position 14 of SEQ ID NO: 1 is Glu (T14E). In some embodiments, the modification at position 14 of SEQ ID NO: 1 is His (T14H). In some embodiments, the modification at position 65 of SEQ ID NO: 1 is Lys (S65K). In some embodiments, the modification at position 66 of SEQ ID NO: 1 is Gly (K66G). In some embodiments, the modification at position 67 of SEQ ID NO: 1 is His (L67H). In some embodiments, the polypeptide further comprises one or two Gly at the C-terminal end of the polypeptide.

In some embodiments, the polypeptide has a binding affinity to the 53BP1 Tudor domain of 0.5 to 500×10⁻⁹ M.

Also provided herein is a composition comprising the polypeptide in admixture with a carrier, excipient or diluent. Also provided herein a polynucleotide comprising a nucleic acid sequence encoding the polypeptide, as well as an expression vector comprising the polynucleotide.

In another aspect, provided herein is a composition comprising a polypeptide described herein (or a polynucleotide or expression vector thereof) and one or more components of a gene editing system. In some embodiments, the one or more components of the gene editing system comprise: (i) a nuclease capable of generating a double-strand break within a gene locus of a cell; and (ii) a donor polynucleotide. In some embodiments, the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR). In some embodiments, the nuclease comprises a CRISPR nuclease and a single guide RNA (sgRNA) capable of hybridizing to a target sequence within the gene locus, wherein the sgRNA guides the CRISPR nuclease to the target sequence. In some embodiments, the CRISPR nuclease is a Cas protein. In some embodiments, the Cas protein is Cas9 or a high-fidelity variant thereof. In some embodiments, the sgRNA and the CRISPR nuclease are formed in a ribonucleoprotein (RNP) complex. In some embodiments, the sgRNA comprises one or more chemically modified nucleotides. In some embodiments, the modified nucleotide is selected from the group consisting of: a 2′-O-methyl nucleotide, a 2′-O-methyl 3′-phosphorothioate nucleotide, and a 2′-O-methyl 3′-thioPACE nucleotide. In some embodiments, a 5′ end, a 3′ end, or a combination thereof of the modified sgRNA comprises a modified nucleotide. In some embodiments, the donor polynucleotide is comprised in a viral vector, a plasmid, or a single-stranded oligodeoxynucleotide (ssODN). In some embodiments, the donor polynucleotide is comprised in an adeno-associated viral (AAV) vector. In some embodiments, the AAV vector is selected from the group consisting of AAV1, AAV2, AAV3, AAV4, AAV3, AAV5, AAV6, AAV7, AAV8, AAV9, AAV10, AAV11, and AAV12. In some embodiments, the AAV vector is an AAV6 vector.

In another aspect, provided herein is a host cell comprising a polypeptide described herein (or a polynucleotide or expression vector thereof). In some embodiments, the host is a mammal. In some embodiments, the mammal is a human. In some embodiments, the cell is a primary cell. In some embodiments, the primary cell is selected from the group consisting of a primary blood cell and a primary mesenchymal cell. In some embodiments, the primary cell is selected from the group consisting of a primary stem cell, primary progenitor cell, and primary somatic cell. In some embodiments, the primary stem cell is selected from the group consisting of an embryonic stem cell, induced pluripotent stem cell, hematopoietic stem cell, mesenchymal stem cell, neural stem cell, and organ stem cell. In some embodiments, the primary progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell. In some embodiments, the primary somatic cell is selected from the group consisting of a fibroblast, a hepatocyte, a heart cell, a liver cell, a pancreatic cell, a muscle cell, a skin cell, a blood cell, a neural cell, and an immune cell. In some embodiments, the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell. In some embodiments, the primary cell is a CD34+ hematopoietic stem or progenitor cell.

In another aspect, provided herein is a kit comprising a polypeptide described herein (or a polynucleotide or expression vector thereof) and one or more components of a gene editing system. In some embodiments, the one or more components of a gene editing system comprise: (i) a nuclease capable of generating a double-strand break within a gene locus of a cell; and (ii) a donor polynucleotide. In some embodiments, the kit further comprises an inhibitor of DNA-dependent protein kinase catalytic subunit (DNA-PKcs). In some embodiments, the DNA-PKcs inhibitor is selected from the group consisting of AZD7648, M3814/nedesertib, CC-115 and BAY-8400.

In another aspect, provided herein is a method of increasing homologous recombination in a cell comprising administering a polypeptide described herein. In some embodiments, the method further comprises introducing to the cell one or more components of a gene editing system. In some embodiments, the one or more components of the gene editing system comprise: (i) a nuclease capable of generating a double-strand break within a gene locus of a cell; and (ii) a donor polynucleotide.

In another aspect, provided herein is a method of stably integrating an exogenous polynucleotide sequence into the genome of a cell, the method comprising introducing into the cell: (a) a nuclease capable of generating a double-strand break within a gene locus of the cell; (b) a donor polynucleotide; and a polypeptide described herein, whereupon generation of a double-strand break within the gene locus by the nuclease, the donor polynucleotide sequence is integrated into the gene locus by homology directed repair (HDR) to yield gene-edited cells. In some embodiments, the method is performed ex vivo. In some embodiments, the gene locus of the cell comprises one or more mutations associated with a disease or encodes an aberrant protein. In some embodiments, integration of the donor polynucleotide sequence into the host cell genome corrects a mutation in the cell that is associated with a disease. In some embodiments, integration of the donor polynucleotide sequence replaces a mutant allele in the cell with a wild-type allele. In some embodiments, the disease is selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. In some embodiments, the hemoglobinopathy is sickle cell disease, α-thalassemia, β-thalassemia, or δ-thalassemia. In some embodiments, the method further comprises administering the gene-edited cells to a patient in need thereof. In some embodiments, the administering comprises an autologous transplant of the gene-edited cells to the patient. In some embodiments, the administering comprises an allogeneic transplant of the gene-edited cells to the patient.

In another aspect, provided herein is a method of identifying a variant polypeptide of a 53BP1-inhibiting polypeptide having the amino acid sequence of SEQ ID NO:1, wherein the variant polypeptide is capable of mediating improved homology-directed repair (HDR)-mediated integration of a donor polynucleotide, the method comprising the steps of:
(a) contacting a CD34+ hematopoietic stem and progenitor cell (HSPC) population with: (i) a control polypeptide comprising the amino acid sequence of SEQ ID NO:1; (ii) one or more variant polypeptides, wherein each variant polypeptide comprises an amino acid sequence of SEQ ID NO: 1 having one or more modifications relative to SEQ ID NO: 1, wherein the one or more modifications comprise a modification at position 67 of SEQ ID NO: 1, a modification at position 68 of SEQ ID NO: 1, or a combination thereof; (iii) a nuclease capable of generating a double-strand break within a gene locus of the HSPCs; and (iv) a donor polynucleotide comprising a reporter gene;
(b) isolating (i) a first subpopulation of contacted HSPCs that expresses at least a threshold amount of the reporter gene; and (ii) a second subpopulation of contacted HSPCs that does not express the threshold amount of the reporter gene; and
(c) measuring the relative abundance of control and variant polypeptides in both the first and second HSPC subpopulations, respectively.

In some embodiments, a variant polypeptide is identified as capable of mediating improved homology-directed repair (HDR)-mediated integration relative to the control polypeptide if: (i) the variant polypeptide is more abundant than the control polypeptide in the first subpopulation; and/or (ii) the variant: control polypeptide ratio of the first subpopulation is higher than the variant:control polypeptide ratio of the second subpopulation. In some embodiments, each of the one or more variant polypeptides further comprises a modification at position 12 of SEQ ID NO: 1, a modification at position 14 of SEQ ID NO:1, or a combination thereof. In some embodiments, each of the one or more variant polypeptides further comprises a modification at position 65 of SEQ ID NO:1, a modification at position 66 of SEQ ID NO:1, or a combination thereof. In some embodiments, contacting of the first and second HSPC populations with the control and variant polypeptides, respectively, comprises contacting the first and second HSPC populations with lentiviral vectors encoding the control and variant polypeptides, respectively. In some embodiments, measuring the relative abundance of control and variant polypeptides comprises sequencing of the lentiviral vectors encoding the control and variant polypeptides. In some embodiments, each modification of the variant polypeptide comprises a single amino acid substitution. In some embodiments, each lentiviral vector encoding a variant polypeptide comprises a polynucleotide comprising an NNK codon encoding a modification of the variant polypeptide.

In some embodiments, the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR). In some embodiments, the nuclease comprises a CRISPR nuclease and a single guide RNA (sgRNA) capable of hybridizing to a target sequence within the gene locus, wherein the sgRNA guides the CRISPR nuclease to the target sequence. In some embodiments, the sgRNA and the CRISPR nuclease are formed in a ribonucleoprotein (RNP) complex. In some embodiments, the donor polynucleotide is comprised in a viral vector, a plasmid, or a single-stranded oligodeoxynucleotide (ssODN). In some embodiments, the donor polynucleotide is comprised in an adeno-associated viral (AAV) vector. In some embodiments, the method further comprises isolating a variant polypeptide having improved HDR activity from the second HSPC population.

### BRIEF DESCRIPTION OF THE FIGURES

FIG. 1 provides a schematic outlining the impact of inhibiting the DNA repair enzyme, 53BP1, can have on various editing outcomes that can occur after a nuclease-mediated site-specific double strand break (DSB). NHEJ: non-homologous end joining; MMEJ: microhomology-mediated end joining; HDR: homology-directed repair.

FIG. 2 provides a schematic outlining the development of a lentiviral-based pooled functional screening system in HSPCs used to identify HDR-enhancing proteins for a Cas9-mediated cut site of interest.

FIG. 3 provides sequences (A) and results (B) relating to validation of a functional screening system for identification of improved variants of i53. Sequences included: 153 (positive control), a previously reported dead mutant of i53 (“DM”=P67L L70V, negative control), and three mutants of i53 (“mut1”=L2Q, “mut2”=D64E, and “mut3”=L62Q) that were been previously reported to have decreased (but detectable) binding to the 53BP1 Tudor domain relative to i53.

FIG. 4 provides a schematic of the binding interface between i53 and 53BP1 Tudor domain at positions 67 and 68 of i53, which were targeted for mutagenesis (A). Results of a lentiviral-based pooled functional screen identifying single amino acid mutants of i53 that displayed enhanced HDR boosting capabilities relative to i53 at the HBB locus in HPSCs (B).

FIG. 5 provides results demonstrating validation of the two top hits from functional screening of an NNK library at residues 67 and 68 (L67R, L67H) via lentiviral expression.

FIG. 6 provides results from size exclusion chromatography demonstrating binding of i53 variants L67R and L67H with the 53BP1 Tudor domain.

FIG. 7 provides results from Biolayer Interferometry (BLI) demonstrating binding kinetics of i53 mutants with immobilized 53BP1 Tudor domain.

FIG. 8 provides structures obtained from crystallography studies demonstrating i53 variants L67R and L67H complexed to the 53bp1 Tudor domain.

FIG. 9 provides a schematic detailing the incorporation of purified i53 variants identified from functional screening as HDR boosters into an HSPC-based gene editing platform.

FIG. 10 provides results of editing of CD34+ HSPCs using an HBB-UBC-GFP AAV donor and purified L67R and L67H i53 variants, relative to i53 and the i53 DM mutant.

FIG. 11 provides results of HDR rates (A) and DNA repair outcomes (B) following editing of CD34+ HSPC cells using an HBB-SNP AAV donor (designed to correct the sequence encoding the E6V mutation in sickle cell disease) and purified L67R and L67H i53 variants, relative to i53 and the i53 DM mutant.

FIG. 12 provides dose response curves of purified i53 variants L67R and L67H relative to the parental control i53 following editing of CD34+ HSPCs using an HBB-UBC-GFP AAV donor.

FIG. 13 provides dose response curves of purified i53 variants L67R and L67H relative to the parental control i53 following editing of CD34+ HSPCs using an HBB-SNP AAV donor.

FIG. 14 provides HDR rates following editing of CD34+ HSPC cells using an MOI titration of an HBB-SNP AAV donor and purified i53 variant L67R.

FIG. 15 provides results of a lentiviral-based pooled functional screen of combinatorial libraries targeting residues at the 53bp1/i53 interface.

FIG. 16 provides results demonstrating validation of the top hits from functional screening of a combinatorial library at residues 67 and 68 via pooled lentiviral expression.

FIG. 17 provides dose response curves of purified i53 variants L67R, L67H and two representative purified double mutants of i53 (L67K H68F and L67H H68Y) following editing of CD34+ HSPC cells using an HBB-SNP AAV donor.

FIG. 18 provides additional HDR boosting mutants of i53 identified by iterating on the L67R i53 variant and screening combinatorial libraries targeting additional residues at the 53bp1/i53 interface.

FIG. 19 provides additional HDR boosting mutants of i53 were identified by iterating on the L67H i53 variant and screening combinatorial libraries targeting additional residues at the 53bp1/i53 interface.

FIG. 20 provides validation of the top hits from the combinatorial library at residues 12 and 14 using L67H as parent via pooled lentiviral expression.

FIG. 21 provides additional HDR boosting mutants of i53 identified by iterating on the L67H.H68Y i53 variant and screening combinatorial libraries targeting additional residues at the 53bp1/i53 interface.

FIG. 22 provides additional HDR boosting mutants of i53 identified by iterating on the L67K.H68F i53 variant and screening combinatorial libraries targeting additional residues at the 53bp1/i53 interface.

FIG. 23 provides validation of the top hits from the combinatorial library at residues 65 and 66 using T12Y.T14E.L67R as parent via pooled lentiviral expression.

FIG. 24 provides GFP knock-in to CD34+ HSPC cells using three representative purified “hit” mutants of i53 (T12Y.T14E.L67R, T12V.T14H.L67H, and T12Y.T14E.S65K.K66G.L67R) and an AAV donor targeted to the HBB, HBA, CCR5 and Il2RG12 locus, respectively.

FIG. 25 provides editing of CD34+ HSPC cells from three donors using three representative purified “hit” mutants of i53 identified in the above screens (T12Y.T14E.L67R, T12V.T14H.L67H, and T12Y.T14E.S65K.K66G.L67R) and an AAV donor template designed to correct the mutation encoding E6V in sickle cell disease.

FIG. 26 provides dose response curves of purified “hit” mutants of i53 (L67R, L67H.H68Y, T12Y.T14E.L67R, T12V.T14H.L67H, and T12Y.T14E.S65K.K66G.L67R) relative to parental i53 using an AAV donor template designed to correct the mutation encoding E6V in sickle cell disease.

FIG. 27 provides results of Biolayer Interferometry (BLI) analysis to measure binding kinetics of additional i53 mutants with immobilized 53BP1 Tudor domain.

FIG. 28 provides characterization of the stability and poly-reactivity of a subset of purified i53 variant proteins using dynamic light scattering (DLS).

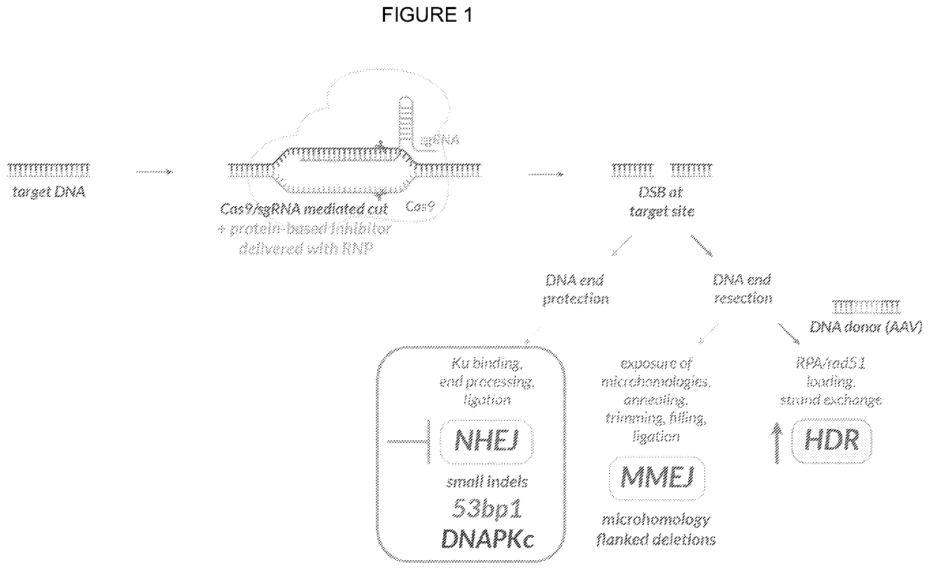

### DETAILED DESCRIPTION

The present disclosure provides compositions and methods for increasing homology-directed repair using inhibitors of 53BP1.

In certain gene-editing applications, the innate cellular repair mechanism homology-directed repair (HDR) enables precise templated repair of nuclease-induced double strand breaks (DSBs) in cells (e.g., hematopoietic stem and progenitor cells (HSPCs) using a donor sequence, which can be delivered via a vector such as adeno-associated virus (e.g., AAV6), to correct, replace, or insert genes anywhere in the genome. However, other DSB repair pathways exist within cells, and re-ligation of the DSB ends can also occur through pathways such as non-homologous end joining (NHEJ) and microhomology-mediated end joining (MMEJ). NHEJ and MMEJ compete with HDR and can yield undesired insertions and deletions (indels) in addition to the desired donor-templated outcome. Ratios of HDR over indel outcomes can be improved using increased levels of donor template; however, high titers of donor vectors such as AAV6 can induce DNA damage response (DDR) pathways within cells, limiting the proliferation, yield, and engraftment of edited cells in ex vivo editing applications. An alternative pathway for improving HDR without increasing viral vector titers (or alternatively, enabling a decrease in viral vector titer while maintaining desired HDR levels) is through the inhibition of key DNA repair enzymes involved in NHEJ and MMEJ-mediated repair. The polypeptides described herein have been identified to inhibit the recruitment of 53BP1 specifically and efficiently, a key enzyme involved in NHEJ repair, to Cas9-mediated DSBs in order to improve HDR and decrease NHEJ in HDR-based gene editing applications.

### Definitions

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the disclosure pertains.

As used herein, the singular forms “a,” “an,” and “the” include plural referents unless otherwise noted or contradicted within the context of the disclosure.

As used herein, “or” means “and/or” unless otherwise noted or contradicted within the context of the disclosure.

As used herein “comprising,” “including,” “containing,” “having” and the like are intended to be open-ended and inclusive such that they do not exclude additional, unrecited elements unless otherwise noted.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

“Ranges”: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Homologous recombination” and “HR” refer to a type of genetic recombination in which DNA strands of similar or identical nucleotide sequences are exchanged. HR can be used by cells to repair DNA double-strand breaks (DSB) by the following general steps. HR is initiated when the DSB is resected by nucleases and helicases, generating 3′ single-stranded DNA (ssDNA) overhangs onto which the RAD51 recombinase assembles as a nucleoprotein filament. This structure can invade homologous duplex DNA, which is used as a template for repair DNA synthesis. The resulting intermediates can be differentially metabolized to produce crossover or non-crossover products (San Filippo et al., Annu. Rev. Biochem. 2008. 77:229-57). Following a double-strand break, sections around the 5′ ends of the break are resected by nucleases and helicases to generate 3′ single-stranded DNA overhangs onto which RAD51 recombinase assembles as a nucleoprotein filament. This structure then invades homologous duplex DNA which is used as a template for DNA repair synthesis. The resulting intermediates can be metabolized to yield non-crossover products thereby restoring the damaged DNA molecule as it existed before the double-strand break. The terms also include recombination using single-stranded oligonucleotides (ssODNs), in particular recombination using single-stranded oligonucleotides (ssODNs) requiring resection and which may be activated by 53BP1 inhibitors.

“HDR”, or “homology-directed repair,” as used herein, refers to the process of repairing DNA damage using a homologous nucleic acid (e.g., an endogenous homologous sequence, e.g., a sister chromatid, or an exogenous nucleic acid, e.g., a template nucleic acid such as a donor polynucleotide described herein). Canonical HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded. This process can be induced is by a number of site-specific nuclease systems that create a double-strand break, such as meganucleases, zinc finger nucleases (ZFNs), transcription activator-like effector nucleases (TALENs), and the CRISPR-Cas gene editing systems. In particular embodiments, HDR involves double-stranded breaks induced by CRISPR-Cas nuclease, e.g. Cas9.

The terms “patient,” “subject,” “individual,” and the like are used interchangeably herein, and refer to any animal amenable to the methods described herein. In certain non-limiting embodiments, the patient, subject or individual is a human.

The term “diagnosis”, or “diagnosing” as used herein refers to the process of identifying a disease, such as cancer, by its signs, symptoms, and/or results of various tests. A conclusion reached through such a process is a diagnosis. Forms of testing commonly performed include blood tests, medical imaging, urinalysis, biopsy, and the like.

The term “therapeutically effective amount”, or simply “effective amount” refers to the amount of an agent or composition (e.g., composition comprising an agent) that will elicit a biological or medical response of a tissue, system, or subject that is being sought by the researcher, veterinarian, medical doctor or other clinician. The term “therapeutically effective amount” includes that amount of an agent, or a composition comprising an agent, that, when administered, is sufficient to prevent development of, or alleviate to some extent, one or more of the signs or symptoms of the disorder or disease (e.g., hematological or solid tumor) being treated. The therapeutically effective amount will vary depending on the composition, the disease and its severity and the age, weight, etc., of the subject to be treated.

To “treat” a disease as the term is used herein, means to decrease or reduce the frequency or severity of at least one sign or symptom of a disease or disorder experienced by a subject. In one example, a therapy (e.g., administration of a therapeutic agent of the present disclosure) treats a disease or condition by decreasing one or more signs or symptoms associated with the disease or condition, for example as compared to the response in the absence of the therapy. For example, administration of a therapeutic agent may provide an anti-tumor effect that decreases one or more signs or symptoms associated with cancer.

As used herein, the term “administration” means to provide or give a subject one or more agents, such as an agent that treats one or more signs or symptoms associated with a condition/disorder or disease including but not limited to cancer (e.g., lymphoma), viral infection, bacterial infection, etc., by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes. Administration “in combination with” one or more further therapeutic agents includes simultaneous (concurrent) and sequential administration in any order.

The term “pharmaceutically acceptable”, as used herein, refers to a material, including but not limited, to a salt, carrier, or diluent, which does not abrogate the biological activity or properties of the compound, and is relatively non-toxic, i.e., the material may be administered to an individual without causing undesirable biological effects or interacting in a deleterious manner with any of the components of the composition in which it is contained. The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more agents, such as one or more modulatory agents. In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol, or the like as a vehicle. In addition to biologically neutral carriers, pharmaceutical agents to be administered can contain minor amounts of non-toxic auxiliary substances, such as wetting or emulsifying agents, preservatives, and pH buffering agents and the like, for example sodium acetate or sorbitan monolaurate, sodium lactate, potassium chloride, calcium chloride, and triethanolamine oleate.

As used herein, the term “autologous” is meant to refer to any material derived from an individual which is later to be re-introduced into the same individual.

As used herein, the term “allogeneic” refers to material derived from an individual which is later introduced into a different individual of the same species.

As used herein, the term “donor polynucleotide” refers to a polynucleotide sequence comprising a donor sequence (including, for example, coding, non-coding and/or regulatory sequences) that is flanked by a 5′ and 3′ homology arm, respectively, having sequence similarity to a targeted nucleic acid sequence. In some embodiments, the donor polynucleotide can be comprised in a vector, for example, a circular plasmid, linear, or made to be linear through a cleavage process.

As used herein, the term “Cas molecule” refers to a Cas polypeptide, Cas protein or a nucleic acid encoding a Cas9 polypeptide. A “Cas polypeptide” or “Cas protein” is a polypeptide that can interact with a gRNA molecule and, in concert with the gRNA molecule, localize to a site comprising a target domain and, in certain embodiments, a PAM sequence. Cas molecules include both naturally occurring Cas molecules and Cas molecules and engineered, altered, or modified Cas molecules or Cas polypeptides that differ, e.g., by at least one amino acid residue, from a reference sequence, e.g., the most similar naturally occurring Cas molecule. (The terms altered, engineered or modified, as used in this context, refer merely to a difference from a reference or naturally occurring sequence, and impose no specific process or origin limitations.) A Cas molecule may be a Cas9 polypeptide or a nucleic acid encoding a Cas9 polypeptide. A Cas molecule may be a nuclease (an enzyme that cleaves both strands of a double-stranded nucleic acid), a nickase (an enzyme that cleaves one strand of a double-stranded nucleic acid), or an enzymatically inactive (or dead) Cas molecule. Exemplary Cas molecules include high-fidelity Cas variants having improved on-target specificity and reduced off-target activity. Examples of high-fidelity Cas9 variants include but are not limited to those described in PCT Publication Nos. WO/2018/068053 and WO/2019/074542, each of which is herein incorporated by reference in its entirety.

As used herein, the terms “gRNA molecule” and “gRNA” refer to a guide RNA which is capable of targeting a Cas molecule to a target nucleic acid. In one embodiment, the term “gRNA molecule” refers to a guide ribonucleic acid. In another embodiment, the term “gRNA molecule” refers to a nucleic acid encoding a gRNA. In one embodiment, a gRNA molecule is non-naturally occurring. In one embodiment, a gRNA molecule is a synthetic gRNA molecule. The phrase “gRNA molecule” as used herein encompasses single guide RNA molecules, which comprise on a single nucleic acid molecule a first nucleotide sequence that is complementary to a target nucleic acid (e.g. within a gene locus) and a second nucleotide sequence that interacts with a CRISPR/Cas polypeptide, wherein the modified sgRNA guides the CRISPR/Cas polypeptide to the target nucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Expression cassette” refers to a nucleic acid comprising expression control sequences operatively linked to a nucleic acid encoding a transcript or polypeptide to be expressed. An expression cassette comprises sufficient cis-acting elements for expression; other elements for expression can be supplied by the host cell or in an in vitro expression system. Expression cassettes can be a component of a vector such as a cosmid, a plasmid (e.g., naked or contained in a liposome), or a virus (e.g., lentivirus, retrovirus, adenovirus, and adeno-associated virus). An expression cassette can be in a host cell.

An “amino acid” includes natural and synthetic amino acids, and both D and L amino acids.

As used herein, a “gene editing system” is a system for the targeting and editing of nucleic acid sequences (e.g. within a genetic locus). Non-limiting, exemplary systems for gene editing include a CRISPR (Clustered Regulatory Interspaced Short Palindromic Repeat) system, a Zinc-Finger Nuclease (ZFN) system and a (Transcription Activator Like Effector Nucleases (TALEN) system. The components of such systems and methods of use are known in the art. An exemplary CRISPR system is disclosed in U.S. Pat. No. 11,193,141 which is incorporated herein by reference in its entirety.

A “polypeptide fragment” is a portion of a polypeptide that includes a region substantially identical to a polypeptide of the present disclosure and has at least 60%, 70%, 80%, 90%, 95%, 98%, 99% or more of the binding activity for the Tudor domain of 53BP1 as the polypeptide of which it is a fragment. By way of example, but not limitation, a polypeptide fragment can include at least 1, 5, 10, 15, 20, 25, 30, 40 or more fewer amino acids than the full-length polypeptide. For example, a polypeptide of the present disclosure can be a polypeptide fragment of SEQ ID NO: 1 or of a modified sequence thereof as described herein.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared. In certain embodiments, default parameters are used.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

A “modification” to a polypeptide of the present disclosure can include substitutions, insertions, deletions and chemical modifications. Techniques for the preparation of such polypeptide and polynucleotides encoding the same are known in the art.

“Binding affinity” or “binding” to the Tudor domain (residues 1484-1603) of 53BP1 can be measured by suitable assays in the art or by measuring 53BP1 recruitment to DSB sites as described in Canny, et al., “Inhibition of 53BP1 favors homology-dependent DNA repair and increases CRISPR-Cas9 genome-editing efficiency,” Nat Biotechnol. 36 (1): 95-102 (2018) which is incorporated herein by reference in its entirety.

It should be understood that reference to a “position” in a polypeptide refers to the amino acid residue corresponding to the position in the reference polypeptide sequence. For example, position 68 refers to the position of the polypeptide of the present disclosure corresponding to position 68 in SEQ ID NO: 1 regardless of whether the position in the polypeptide of the present disclosure is the 68th amino acid position.

### Polypeptides

In some embodiments, a polypeptide is provided that comprises an amino acid sequence having at least 60% identity to the amino acid sequence of SEQ ID NO: 1 and one or more modifications relative to SEQ ID NO: 1. In certain embodiments, the one or more modifications can include a modification at position 67 of SEQ ID NO: 1, a modification at position 68 of SEQ ID NO: 1, or a combination thereof. In certain embodiments, the polypeptide comprises a modification at position 67. In certain embodiments, the polypeptide comprises a modification at position 68. In certain embodiments, the polypeptide comprises a modification at position 67 and a modification at position 68. In certain embodiments, the polypeptide comprises one or more additional modifications relative to the amino acid sequence of SEQ ID NO: 1.

In any of the foregoing embodiments, the polypeptide can comprise an amino acid sequence having at least 60%, at least 70%, at least 75%, at least 80%, at least 85%, at least 90%, at least 95%, at least 98%, at least 99% or more identity to the amino acid sequence of SEQ ID NO: 1, and includes one or more modifications relative to SEQ ID NO:1.

In some embodiments, a polypeptide is provided that comprises the sequence of SEQ ID NO: 1 having one or more modifications relative to SEQ ID NO: 1, wherein the one or more modifications can include a modification at position 67 of SEQ ID NO: 1, a modification at position 68 of SEQ ID NO: 1, or a combination thereof. In certain embodiments, the polypeptide comprises a modification at position 67. In certain embodiments, the polypeptide comprises a modification at position 68. In certain embodiments, the polypeptide comprises a modification at position 67 and a modification at position 68. In certain embodiments, the polypeptide comprises one or more additional modifications relative to the amino acid sequence of SEQ ID NO: 1.

In any of the foregoing embodiments, the modification at position 67 of SEQ ID NO: 1 can be: (a) an Arg (L67R), (b) a His (L67H), (c) a Lys (L67K) provided that the polypeptide further comprises a modification at position 68 (L67K, H68*), (d) a Ser (L67S), (e) a Thr (L67T), (f) a Gln (L67Q), or (g) an Asn (L67N). In certain embodiments, the modification at position 67 of SEQ ID NO: 1 can be: (a) an Arg (L67R), (b) a His (L67H), (c) a Ser (L67S), (d) a Thr (L67T), (e) a Gln (L67Q), or (f) an Asn (L67N). In certain embodiments, the modification at position 67 of SEQ ID NO: 1 can be: (a) an Arg (L67R), (b) a His (L67H), or (c) a Lys (L67K), provided that the polypeptide further comprises a modification at position 68 (L67K, H68*). In certain embodiments, the modification at position 67 of SEQ ID NO: 1 can be: (a) an Arg (L67R), or (b) a His (L67H). In certain embodiments, the modification at position 67 of SEQ ID NO: 1 is not L67K.

In any of the foregoing embodiments, the modification at position 68 of SEQ ID NO: 1 can be: (a) a Trp (H68W), (b) a Tyr (H68Y), or a Phe (H68F).

In any of the foregoing embodiments, the one or more modifications can be: (a) an Arg at position 67 of SEQ ID NO: 1 (L67R), and a Trp at position 68 of SEQ ID NO: 1 (H68W); (b) a His at position 67 of SEQ ID NO: 1 (L67H), and a Tyr at position 68 of SEQ ID NO: 1 (H68Y); (c) a His at position 67 of SEQ ID NO: 1 (L67H), and a Phe at position 68 of SEQ ID NO: 1 (H68F), (d) a His at position 67 of SEQ ID NO: 1 (L67H), and a Trp at position 68 of SEQ ID NO: 1 (H68W); (e) an Arg at position 67 of SEQ ID NO: 1 (L67R), and a Tyr at position 68 of SEQ ID NO: 1 (H68Y); (f) an Arg at position 67 of SEQ ID NO: 1 (L67R), and a Phe at position 68 of SEQ ID NO: 1 (H68F); (g) a Ser at position 67 of SEQ ID NO: 1 (L67S), and a Phe at position 68 of SEQ ID NO: 1 (H68F); (h) a Thr at position 67 of SEQ ID NO: 1 (L67T), and a Trp at position 68 of SEQ ID NO: 1 (H68W); (i) a Ser at position 67 of SEQ ID NO: 1 (L67S), and a Trp at position 68 of SEQ ID NO: 1 (H68W); (j) a Gln at position 67 of SEQ ID NO: 1 (L67Q), and a Tyr at position 68 of SEQ ID NO: 1 (H68Y); (k) an Asn at position 67 of SEQ ID NO: 1 (L67N), and a Tyr at position 68 of SEQ ID NO: 1 (H68Y); (1) an Asn at position 67 of SEQ ID NO: 1 (L67N), and a Trp at position 68 of SEQ ID NO: 1 (H68W); or (m) a Lys at position 67 of SEQ ID NO: 1 (L67K), and a Tyr at position 68 of SEQ ID NO: 1 (H68Y).

In some embodiments, a polypeptide is provided comprising the sequence of SEQ ID NO: 2. In some embodiments, a polypeptide is provided consisting of the sequence of SEQ ID NO: 2.

In some embodiments, a polypeptide is provided comprising the sequence of SEQ ID NO: 3. In some embodiments, a polypeptide is provided consisting of the sequence of SEQ ID NO: 3.

In some embodiments, a polypeptide is provided comprising the sequence of SEQ ID NO: 4. In some embodiments, a polypeptide is provided consisting of the sequence of SEQ ID NO: 4.

In some embodiments, a polypeptide is provided comprising the sequence of SEQ ID NO: 5. In some embodiments, a polypeptide is provided consisting of the sequence of SEQ ID NO: 5.

In some embodiments, a polypeptide is provided comprising the sequence of SEQ ID NO: 6. In some embodiments, a polypeptide is provided consisting of the sequence of SEQ ID NO: 6.

In some embodiments, a polypeptide is provided comprising the sequence of SEQ ID NO: 7. In some embodiments, a polypeptide is provided consisting of the sequence of SEQ ID NO: 7.

In some embodiments, a polypeptide is provided comprising the sequence of SEQ ID NO: 8. In some embodiments, a polypeptide is provided consisting of the sequence of SEQ ID NO: 8.

In some embodiments, a polypeptide is provided comprising the sequence of SEQ ID NO: 9. In some embodiments, a polypeptide is provided consisting of the sequence of SEQ ID NO: 9.

In some embodiments, a polypeptide is provided comprising the sequence of SEQ ID NO: 10. In some embodiments, a polypeptide is provided consisting of the sequence of SEQ ID NO: 10.

In some embodiments, a polypeptide is provided comprising the sequence of SEQ ID NO: 11. In some embodiments, a polypeptide is provided consisting of the sequence of SEQ ID NO: 11.

In some embodiments, a polypeptide is provided comprising the sequence of SEQ ID NO: 12. In some embodiments, a polypeptide is provided consisting of the sequence of SEQ ID NO: 12.

In some embodiments, a polypeptide is provided comprising the sequence of SEQ ID NO: 13. In some embodiments, a polypeptide is provided consisting of the sequence of SEQ ID NO: 13.

In some embodiments, a polypeptide is provided comprising the sequence of SEQ ID NO: 14. In some embodiments, a polypeptide is provided consisting of the sequence of SEQ ID NO: 14.

In some embodiments, a polypeptide is provided comprising the sequence of SEQ ID NO: 15. In some embodiments, a polypeptide is provided consisting of the sequence of SEQ ID NO: 15.

In some embodiments, a polypeptide is provided comprising the sequence of SEQ ID NO: 16. In some embodiments, a polypeptide is provided consisting of the sequence of SEQ ID NO: 16.

In some embodiments, a polypeptide is provided comprising the sequence of SEQ ID NO: 17. In some embodiments, a polypeptide is provided consisting of the sequence of SEQ ID NO: 17.

In some embodiments, a polypeptide is provided comprising the sequence of SEQ ID NO: 18. In some embodiments, a polypeptide is provided consisting of the sequence of SEQ ID NO: 18.

In some embodiments, a polypeptide is provided comprising the sequence of SEQ ID NO: 19. In some embodiments, a polypeptide is provided consisting of the sequence of SEQ ID NO: 19.

In some embodiments, a polypeptide is provided comprising the sequence of SEQ ID NO: 20. In some embodiments, a polypeptide is provided consisting of the sequence of SEQ ID NO: 20.

In some embodiments, a polypeptide is provided comprising the sequence of SEQ ID NO: 21. In some embodiments, a polypeptide is provided consisting of the sequence of SEQ ID NO: 21.

In some embodiments, a polypeptide is provided comprising the sequence of SEQ ID NO: 22. In some embodiments, a polypeptide is provided consisting of the sequence of SEQ ID NO: 22.

In some embodiments, a polypeptide is provided comprising the sequence of SEQ ID NO: 23. In some embodiments, a polypeptide is provided consisting of the sequence of SEQ ID NO: 23.

In some embodiments, a polypeptide is provided comprising the sequence of SEQ ID NO: 24. In some embodiments, a polypeptide is provided consisting of the sequence of SEQ ID NO: 24.

In some embodiments, a polypeptide is provided comprising the sequence of SEQ ID NO: 25. In some embodiments, a polypeptide is provided consisting of the sequence of SEQ ID NO: 25.

In some embodiments, a polypeptide is provided comprising the sequence of SEQ ID NO: 26. In some embodiments, a polypeptide is provided consisting of the sequence of SEQ ID NO: 26.

In some embodiments, a polypeptide is provided comprising the sequence of SEQ ID NO: 27. In some embodiments, a polypeptide is provided consisting of the sequence of SEQ ID NO: 27.

In some embodiments, a polypeptide is provided comprising the sequence of SEQ ID NO: 28. In some embodiments, a polypeptide is provided consisting of the sequence of SEQ ID NO: 28.

In some embodiments, a polypeptide is provided comprising the sequence of SEQ ID NO: 29. In some embodiments, a polypeptide is provided consisting of the sequence of SEQ ID NO: 29.

In some embodiments, a polypeptide is provided comprising the sequence of SEQ ID NO: 30. In some embodiments, a polypeptide is provided consisting of the sequence of SEQ ID NO: 30.

In some embodiments, a polypeptide is provided comprising the sequence of SEQ ID NO: 31. In some embodiments, a polypeptide is provided consisting of the sequence of SEQ ID NO: 31.

In some embodiments, a polypeptide is provided comprising the sequence of SEQ ID NO: 32. In some embodiments, a polypeptide is provided consisting of the sequence of SEQ ID NO: 32.

In some embodiments, a polypeptide is provided comprising the sequence of SEQ ID NO: 33. In some embodiments, a polypeptide is provided consisting of the sequence of SEQ ID NO: 33.

In some embodiments, a polypeptide is provided comprising the sequence of SEQ ID NO: 34. In some embodiments, a polypeptide is provided consisting of the sequence of SEQ ID NO: 34.

In some embodiments, a polypeptide is provided comprising the sequence of SEQ ID NO: 35. In some embodiments, a polypeptide is provided consisting of the sequence of SEQ ID NO: 35.

In some embodiments, a polypeptide is provided comprising the sequence of SEQ ID NO: 36. In some embodiments, a polypeptide is provided consisting of the sequence of SEQ ID NO: 36.

In some embodiments, a polypeptide is provided comprising the sequence of SEQ ID NO: 37. In some embodiments, a polypeptide is provided consisting of the sequence of SEQ ID NO: 37.

In some embodiments, a polypeptide is provided comprising the sequence of SEQ ID NO: 38. In some embodiments, a polypeptide is provided consisting of the sequence of SEQ ID NO: 38.

In some embodiments, a polypeptide is provided comprising the sequence of SEQ ID NO: 39. In some embodiments, a polypeptide is provided consisting of the sequence of SEQ ID NO: 39.

In some embodiments, a polypeptide is provided comprising the sequence of SEQ ID NO: 40. In some embodiments, a polypeptide is provided consisting of the sequence of SEQ ID NO: 40.

In some embodiments, a polypeptide is provided comprising the sequence of SEQ ID NO: 41. In some embodiments, a polypeptide is provided consisting of the sequence of SEQ ID NO: 41.

In some embodiments, a polypeptide is provided comprising the sequence of SEQ ID NO: 42. In some embodiments, a polypeptide is provided consisting of the sequence of SEQ ID NO: 42.

In some embodiments, a polypeptide is provided comprising the sequence of SEQ ID NO: 43. In some embodiments, a polypeptide is provided consisting of the sequence of SEQ ID NO: 43.

In some embodiments, a polypeptide is provided comprising the sequence of SEQ ID NO: 44. In some embodiments, a polypeptide is provided consisting of the sequence of SEQ ID NO: 44.

In some embodiments, a polypeptide is provided comprising the sequence of SEQ ID NO: 45. In some embodiments, a polypeptide is provided consisting of the sequence of SEQ ID NO: 45.

In some embodiments, a polypeptide is provided comprising the sequence of SEQ ID NO: 46. In some embodiments, a polypeptide is provided consisting of the sequence of SEQ ID NO: 46.

In some embodiments, a polypeptide is provided comprising the sequence of SEQ ID NO: 47. In some embodiments, a polypeptide is provided consisting of the sequence of SEQ ID NO: 47.

In some embodiments, a polypeptide is provided comprising the sequence of SEQ ID NO: 48. In some embodiments, a polypeptide is provided consisting of the sequence of SEQ ID NO: 48.

In some embodiments, a polypeptide is provided comprising the sequence of SEQ ID NO: 49. In some embodiments, a polypeptide is provided consisting of the sequence of SEQ ID NO: 49.

In some embodiments, a polypeptide is provided comprising the sequence of SEQ ID NO: 50. In some embodiments, a polypeptide is provided consisting of the sequence of SEQ ID NO: 50.

In some embodiments, a polypeptide is provided comprising the sequence of SEQ ID NO: 51. In some embodiments, a polypeptide is provided consisting of the sequence of SEQ ID NO: 51.

In some embodiments, a polypeptide is provided comprising the sequence of SEQ ID NO: 52. In some embodiments, a polypeptide is provided consisting of the sequence of SEQ ID NO: 52.

In some embodiments, a polypeptide is provided comprising the sequence of SEQ ID NO: 53. In some embodiments, a polypeptide is provided consisting of the sequence of SEQ ID NO: 53.

In some embodiments, a polypeptide is provided comprising the sequence of SEQ ID NO: 54. In some embodiments, a polypeptide is provided consisting of the sequence of SEQ ID NO: 54.

In some embodiments, a polypeptide is provided comprising the sequence of SEQ ID NO: 55. In some embodiments, a polypeptide is provided consisting of the sequence of SEQ ID NO: 55.

In some embodiments, a polypeptide is provided comprising the sequence of SEQ ID NO: 56. In some embodiments, a polypeptide is provided consisting of the sequence of SEQ ID NO: 56.

In some embodiments, a polypeptide is provided comprising the sequence of SEQ ID NO: 57. In some embodiments, a polypeptide is provided consisting of the sequence of SEQ ID NO: 57.

In some embodiments, a polypeptide is provided comprising the sequence of SEQ ID NO: 58. In some embodiments, a polypeptide is provided consisting of the sequence of SEQ ID NO: 58.

In some embodiments, a polypeptide is provided comprising the sequence of SEQ ID NO: 59. In some embodiments, a polypeptide is provided consisting of the sequence of SEQ ID NO: 59.

In some embodiments, a polypeptide is provided comprising the sequence of SEQ ID NO: 60. In some embodiments, a polypeptide is provided consisting of the sequence of SEQ ID NO: 60.

In some embodiments, a polypeptide is provided comprising the sequence of SEQ ID NO: 61. In some embodiments, a polypeptide is provided consisting of the sequence of SEQ ID NO: 61.

In some embodiments, a polypeptide is provided comprising the sequence of SEQ ID NO: 62. In some embodiments, a polypeptide is provided consisting of the sequence of SEQ ID NO: 62.

In some embodiments, a polypeptide is provided comprising the sequence of SEQ ID NO: 63. In some embodiments, a polypeptide is provided consisting of the sequence of SEQ ID NO: 63.

In some embodiments, a polypeptide is provided comprising the sequence of SEQ ID NO: 64. In some embodiments, a polypeptide is provided consisting of the sequence of SEQ ID NO: 64.

In some embodiments, a polypeptide is provided comprising the sequence of SEQ ID NO: 65. In some embodiments, a polypeptide is provided consisting of the sequence of SEQ ID NO: 65.

In some embodiments, a polypeptide is provided comprising the sequence of SEQ ID NO: 66. In some embodiments, a polypeptide is provided consisting of the sequence of SEQ ID NO: 66.

In some embodiments, a polypeptide is provided comprising the sequence of SEQ ID NO: 67. In some embodiments, a polypeptide is provided consisting of the sequence of SEQ ID NO: 67.

In some embodiments, a polypeptide is provided comprising the sequence of SEQ ID NO: 68. In some embodiments, a polypeptide is provided consisting of the sequence of SEQ ID NO: 68.

In some embodiments, a polypeptide is provided comprising the sequence of SEQ ID NO: 69. In some embodiments, a polypeptide is provided consisting of the sequence of SEQ ID NO: 69.

In some embodiments, a polypeptide is provided comprising the sequence of SEQ ID NO: 70. In some embodiments, a polypeptide is provided consisting of the sequence of SEQ ID NO: 70.

In some embodiments, a polypeptide is provided comprising the sequence of SEQ ID NO: 71. In some embodiments, a polypeptide is provided consisting of the sequence of SEQ ID NO: 71.

In some embodiments, a polypeptide is provided comprising the sequence of SEQ ID NO: 72. In some embodiments, a polypeptide is provided consisting of the sequence of SEQ ID NO: 72.

In any of the foregoing embodiments, the polypeptide can further comprise one or more additional modifications relative to the amino acid sequence of SEQ ID NO: 1. By way of example, but not limitation, the polypeptide can further comprise 1, 2, 3, 4, 5, 6, 7, 8, 9, 10, 11, 12, 13, 14, 15, 16, 17, 18, 19, 20 or more additional modifications relative to the amino acid sequence of SEQ ID NO: 1. In some embodiments, the one or more additional modifications selected from the group consisting: (a) a Gln at position 2 of SEQ ID NO: 1 (L2Q), (b) an Ile at position 44 of SEQ ID NO: 1 (A44I), (c) a Gln at position 49 of SEQ ID NO: 1 (S49Q), (d) a Gln at position 62 of SEQ ID NO: 1 (L62Q), (e) a Glu at position 64 of SEQ ID NO: 1 (D64E), (f) a Thr at position 66 of SEQ ID NO: 1 (K66T), (g) a Leu at position 69 of SEQ ID NO: 1 (P69L), (h) a Val at position 70 of SEQ ID NO: 1 (L70V), and combinations thereof. In any of the foregoing embodiments, the one or more additional modifications can include or be selected from the group consisting of: (a) position 12 of SEQ ID NO: 1, (b) position 14 of SEQ ID NO: 1, (c) position 65 of SEQ ID NO: 1, (d) position 66 of SEQ ID NO: 1, (e) position 67 of SEQ ID NO: 1; and (f) combinations thereof. In certain embodiments, the one or more additional modification can include a Tyr at position 12 of SEQ ID NO: 1 (T12Y) and/or a Glu at position 14 of SEQ ID NO: 1 (T14E). In certain embodiments, the one or more additional modifications can include a Val at position 12 of SEQ ID NO: 1 (T12V) and/or a His at position 14 of SEQ ID NO: 1 (T14H). In certain embodiments, the one or more additional modifications can include a Tyr at position 12 of SEQ ID NO: 1 (T12Y) and/or a Glu at position 14 of SEQ ID NO: 1 (T14E). In certain embodiments, the one or more additional modifications can include a Tyr at position 12 of SEQ ID NO: 1 (T12Y) and/or a Glu at position 14 of SEQ ID NO: 1 (T14E), a Lys at position 65 of SEQ ID NO: 1 (S65K) and/or a Gly at position 66 of SEQ ID NO: 1 (K66G). In certain embodiments, the modification at position 67 of SEQ ID NO: 1 is His (L67H). In certain embodiments, the modification at position 67 is Arg (L67R). By way of example, but not limitation, the one or more additional modifications can be made at position(s) selected from the group consisting of positions 2, 4, 6, 8, 10, 11, 12, 14, 44, 46, 47, 48, 49, 62, 63, 64, 65, 66, 69, 70, 71, 72, 73, 74, and combinations thereof, corresponding to the positions of SEQ ID NO: 1.

In any of the foregoing embodiments, the polypeptide can further comprise one or two Gly at the C-terminal end of the polypeptide. It should be understood that the polypeptide can further include additional residues at the N-terminal or C-terminal end of the polypeptide. In any of the foregoing embodiments, the polypeptide includes a Met at an N-terminal end of the polypeptide. Alternatively, in any of the foregoing embodiments, the polypeptide does not include a Met at an N-terminal end of the polypeptide.

In any of the foregoing embodiments, the polypeptide can have a binding affinity to the 53BP1 Tudor domain (residues 1484-1603) of 0.5 to 500×10⁻⁹ M. By way of example, but not limitation, the polypeptide can have a binding affinity to the 53BP1 Tudor domain of 0.5 to 15×10⁻⁹ M, 0.5 to 25×10⁻⁹ M, 0.5 to 50×10⁻⁹ M, 0.5 to 100×10⁻⁹ M, 0.5 to 200×10⁻⁹ M, 1 to 200×10⁻⁹ M, 1 to 300×10⁻⁹ M, 1 to 400×10⁻⁹ M, 1 to 500×10⁻⁹ M, 100 to 300×10⁻⁹ M, 100 to 250×10⁻⁹ M, or 200 to 250×10⁻⁹ M.

It should be understood that, in any of the foregoing embodiments, the polypeptide can be a fragment of a full-length polypeptide and need not contain the full sequence of SEQ ID NO: 1 or a modified form thereof. In any of the foregoing embodiments, the polypeptide or polypeptide fragment can bind the Tudor domain (residues 1484-1603) of 53BP1.

### Fusion Proteins

In some embodiments, a polypeptide of the present disclosure can be further coupled to enzymes, toxins, or other binding proteins, for example, biotin, digoxigenin, GFP, Flag, and fluorescent and/or luminescent substances while retaining binding of the Tudor domain of 53BP1.

Additional amino acids or peptides or substitutions of individual amino acids or peptides may be introduced (in particular at the amino and/or carboxy termini) to obtain fusion proteins by chemical coupling with suitable reagents. Fusion polypeptides may also be prepared by genetic engineering by linking the gene of polypeptide disclosed herein to that of the fusion partner. Bivalent or bispecific polypeptides may be obtained by linking (for example, via an additionally introduced cysteine or positively or negatively charged amino acids at the carboxy terminal ends of the fusion partners) a polypeptide disclosed herein to a polypeptide of the same or a different specificity in a site-specific and covalent manner.

### Polynucleotides

In some embodiments, an isolated polynucleotide encoding the polypeptide of any of the foregoing embodiments is provided. It should be understood that the isolated polynucleotide can be a polynucleotide itself or as incorporated into a vector or cell.

### Vectors

In some embodiments, a vector is provided comprising the polynucleotide encoding the polypeptide of any of the foregoing embodiments. In certain aspects, the vector is an expression vector.

In any of the foregoing embodiments, the vector can be any suitable vector for delivery of the polynucleotide to a host cell. By way of example, but not limitation, such vectors include prokaryotic vectors, viral vectors, or eukaryotic vectors, such as mammalian vectors. Exemplary viral vectors include adenovirus, adeno-associated-virus, retrovirus, herpes virus, lentivirus, poxvirus, and cytomegalovirus.

### Host Cells

In some embodiments, a host cell is provided comprising the polypeptide of any of the foregoing embodiments. The host cells described herein can be used in any of the compositions, kits and methods of the present disclosure.

In some embodiments, a host cell is provided comprising the polypeptide of any of the foregoing embodiments. In any of the foregoing embodiments, the host cell can comprise the polypeptide of any of the foregoing embodiments in admixture with a carrier, excipient or diluent.

In some embodiments, a host cell is provided comprising the polynucleotide of any of the foregoing embodiments.

In some embodiments, a host cell is provided comprising the vector of any of the foregoing embodiments. In certain aspects, the vector is an expression vector.

In an embodiment, an isolated cell or cell lines are provided comprising any of the polypeptides and/or polynucleotides disclosed herein. Cells or cell lines may comprise one or more transcribed and/or translated exogenous sequences that have been stably or transiently introduced into the cells. Non-limiting examples of cells include bacterial cells such as E. coli cells, insect cells, yeast cells, or mammalian cells. In some embodiments, the host cells can be bone marrow cells, umbilical cord blood cells, hematopoietic stem and progenitor cells (HSPCs), peripheral blood CD34⁺ cells, peripheral blood CD34⁺ and CD90⁺ cells, and any combination thereof.

In a preferred embodiment, the host cell is a mammalian cell. In a more preferred embodiment, the mammalian cell is a human cell. In certain embodiments, the host cell is a stem cell. In certain embodiments, the host cell is a primary cell. In certain embodiments, the host cell is selected from the group consisting of a primary blood cell and a primary mesenchymal cell. In certain embodiments, a primary cell is selected from the group consisting of a primary stem cell, primary progenitor cell, and primary somatic cell. In certain embodiments, a primary stem cell is selected from the group consisting of an embryonic stem cell, induced pluripotent stem cell, hematopoietic stem cell, mesenchymal stem cell, neural stem cell, and organ stem cell. In certain embodiments, the progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell. In certain embodiments, a somatic cell is selected from the group consisting of a fibroblast, a hepatocyte, a heart cell, a liver cell, a pancreatic cell, a muscle cell, a skin cell, a blood cell, a neural cell, and an immune cell. In certain embodiments, the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell. In certain embodiments, the primary cell is a CD34⁺ hematopoietic cell or progenitor cell.

In any of the foregoing embodiments, a gene locus of the cell can include one or more mutations associated with a disease or can encode an aberrant protein. In certain embodiments, the disease can be selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. In certain embodiments, the hemoglobinopathy is a sickle cell disease, α-thalassemia, β-thalassemia, or δ-thalassemia.

### Gene Editing Systems

In any of the foregoing embodiments, the host cell can further comprise one or more components of a gene editing system. In any of the foregoing embodiments, the one or more components of the gene editing system can include a nuclease capable of generating a double-strand break within a gene locus of a cell. In any of the foregoing embodiments, the one or more components of the gene editing system can further include a donor polynucleotide.

In any of the foregoing embodiments, the nuclease can include a CRISPR nuclease or polynucleotide or expression vector encoding the CRISPR nuclease, and a single guide RNA (sgRNA) capable of hybridizing to a target sequence within the gene locus or polynucleotide or expression vector encoding the sgRNA. In certain embodiments, the CRISPR nuclease can be a Cas protein. By way of example, but not limitation, the Cas protein can be Cas9 or a high-fidelity variant thereof. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In certain embodiments, the sgRNA and CRISPR nuclease can be formed into a ribonucleoprotein (RNP) complex.

In any of the foregoing embodiments, the sgRNA can include one or more chemically modified nucleotides. In such embodiments, the one or more chemically modified nucleotides can be selected from the group consisting of a 2′-O-methyl nucleotide, a 2′-O-methyl 3′-phosphorothioate nucleotide, and a 2′-O-methyl 3′-thioPACE nucleotide. In any of the foregoing embodiments, a 5′ end, a 3′ end, or a combination thereof of the modified sgRNA comprises a modified nucleotide. In some embodiments, the modified sgRNA comprises one, two, or three consecutive or non-consecutive modified nucleotides at or near the 5′-end of the sgRNA, and/or one, two, or three consecutive or non-consecutive modified nucleotides at or near the 3′-end of the sgRNA. In some embodiments, the modified sgRNA comprises three consecutive modified nucleotides at the 5′-end and three consecutive modified nucleotides at the 3′-end of the sgRNA. The modified sgRNA can be chemically synthesized using methods known in the art.

In any of the foregoing embodiments, the donor polynucleotide can be within a viral vector. In certain aspects, the viral vector can be an adeno-associated viral (AAV) vector including the donor polynucleotide. Other viral vectors useful in methods of gene therapy are known in the art. For example, a construct of the present disclosure can include an alphavirus, herpesvirus, retrovirus, lentivirus, or vaccinia virus. The polynucleotide can be integrated into the genome of the cell by use of systems such as CRISPR system.

The donor polynucleotide sequences generally encode recombinant molecules to be expressed in the cells, e.g., for use in cell therapy. Processing steps of the methods can also or alternatively include all or a portion of cell washing, dilution, selection, isolation, separation, cultivation, stimulation, packaging, and/or formulation. The methods generally allow for the processing, e.g., selection or separation and/or transduction, of cells on a large scale (such as in compositions of volumes greater than or at about 50 mL).

In some embodiments, the gene editing system utilizes site-specific nucleases for knock-out of targeted genomic sequences or knock-in of exogenous sequences, and for transferring exogenous sequences to the cells by viral transduction through the use of recombinant viral vectors.

In some embodiments, the gene editing system includes a nuclease introduced to the cell that is capable of causing a double-strand break near or within a genomic target site, which may be useful for increasing the frequency of HDR and homologous recombination at or near the cleavage site. Gene-editing nucleases useful for the methods provided herein include but are not limited to a TAL-effector DNA binding domain-nuclease fusion protein (TALEN), a site-specific recombinase (for example, serine recombinase or a tyrosine recombinase, integrase (FLP, Cre, lambda integrase) or resolvase; a transposase, a zinc-finger nuclease (ZFN), and a clustered regularly interspaced short palindromic repeats (CRISPR) and CRISPR-associated (Cas) protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In some embodiments the Cas nuclease can be in the form of a protein. In some embodiments, the Cas nuclease can be in the form of a plasmid, thereby allowing a cell that carries this expression construct to then express the Cas nuclease. In other embodiments, the Cas nuclease is pre-complexed with a guide RNA and introduced into the cell as a ribonucleoprotein (RNP). In some embodiments, the Cas nuclease and the guide polynucleotide sequence is introduced into the CD34+ cell through electroporation.

Introduction of the donor polynucleotide can occur through viral transduction using a delivery vector, such as adeno associated virus (AAV). AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally. At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure. A serotype of the viral vector can be selected from the group consisting of AAV1, AAV2, AAV3, AAV4, AAV5, AAV6, AAV7, AAV8, and AAV9. In some embodiments, the serotype is AAV6.

In some embodiments in which a donor polynucleotide is delivered into a cell via a viral vector (for example, an AAV vector), use of a 53BP1-inhibiting polypeptide provided herein can advantageously increase the rate of HDR-mediated integration of the donor sequence for a given multiplicity of infection (MOI) of the viral vector, relative to the rate of integration achieved without the use of the 53BP1-inhibiting polypeptide. Without being bound by theory, it is believed that higher rates of integration achieved with a 53BP1-inhibiting polypeptide provided herein enables lower viral MOIs, and thus reduced DNA damage response (DDR) in response to introduction of the viral vector to the host cell.

### Compositions

In some embodiments, a composition is provided comprises the polypeptide of any of the foregoing embodiments in admixture with a carrier, excipient and/or diluent. In some embodiments, a composition is provided that comprises a polynucleotide of any of the foregoing embodiments in admixture with a carrier, excipient and/or diluent. In some embodiments, a composition is provided that comprises a vector of any of the foregoing embodiments in admixture with a carrier, excipient and/or diluent.

In some embodiments, a composition of the present disclosure can further include an inhibitor of DNA-dependent protein kinase catalytic subunit (DNA-PKcs). In certain embodiment, the DNA-PKcs inhibitor can be AZD7648, M3814/nedesertib, CC-115 or BAY-8400.

In any of the foregoing embodiments, the composition can be a pharmaceutically acceptable composition. In any of the foregoing embodiments, the carrier excipient and/or diluent can be pharmaceutically acceptable. Guidance for preparing pharmaceutical compositions may be found, for example, in Remington: The Science and Practice of Pharmacy, (20th ed.) ed. A. R. Gennaro A. R., 2000, Lippencott Williams & Wilkins.

### Methods of Preparation

Polypeptides of the present disclosure can be prepared by standard methods in the art. By way of example, a polynucleotide encoding the polypeptide can be introduced, for example as part of an expression vector or expression cassette, into a host cell and expressed in the host cell. In certain aspects, the host cell can be incubated under conditions sufficient in a culture medium to yield expression of the polypeptide followed by purification of the polypeptide from the host cell or cell culture medium by methods known in the art.

### Homologous Recombination Methods and Compositions

In some embodiments, a method is provided for increasing homologous recombination in a cell comprising introducing a polypeptide, polynucleotide, or vector of any of the foregoing embodiments to the cell. In any of the foregoing embodiments, the method can further include introducing to the cell one or more components of a gene editing system as described herein.

In some embodiments, the threshold number of cells to be collected from a donor prior to gene editing is about 1×10⁴ to 1×10⁵, 1×10⁵ to 1×10⁶, 1×10⁶ to 1×10⁷ cells/kg or more. In some embodiments, at least about 1×10⁵ to 1×10⁷ cells/kg are collected prior to gene editing. In some embodiments, at least about 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or about 1×10⁸ cells/kg are collected prior to proceeding with gene editing of the collected cells. In some embodiments, the cells to be edited can be any type of host cell described herein.

Methods for introducing polypeptides, nucleic acids, and viral vectors (e.g., viral particles) into a primary cell, target cell, or host cell are known in the art. Any known method can be used to introduce a polypeptide or a nucleic acid (e.g., a nucleotide sequence encoding the DNA nuclease or a modified sgRNA) into a primary cell, e.g., a human primary cell. Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

It should be understood that cells so produced, can be useful in therapeutic applications as gene edited cells.

### Applications of Polypeptides

The polypeptides and/or polynucleotide of the present disclosure may be used in a broad spectrum of applications. The polypeptides and/or polynucleotide may be used for the detection and quantitative determination as well as for the separation and isolation of 53BP1. The present disclosure also provides the use of polypeptides and/or polynucleotides disclosed herein for use as medicaments, particularly for the treatment of a genetic disorder, and for gene and cell therapies as described herein.

The polypeptides and polynucleotides disclosed herein may be used in genomic engineering, epigenomic engineering, genome targeting, and genome editing. The polypeptides and polynucleotides disclosed herein may be used to modify repair pathways, activate or stimulate HR or homology-based genome editing, inhibit 53BP1 recruitment to DSB sites or damaged chromatin in a cell or modulate DNA end resection. In an aspect, the polypeptides and polynucleotides disclosed herein are used in combination with a gene editing system. In some such embodiments, the gene-editing system comprises a nuclease capable of generating a double-strand break within a gene locus of a cell and a donor polynucleotide. In embodiments, the polypeptide and/or polynucleotide is administered in combination with one or more elements or components of a CRISPR system. In embodiments, the polypeptide and/or polynucleotide is administered before, simultaneously or after one or more elements or components of a CRISPR system. In embodiments, the polypeptide and/or polynucleotide is administered before, simultaneously or after one or more elements or components of a CRISPR/Cas system. In embodiments, the polypeptide and/or polynucleotide is administered before, simultaneously or after one or more elements or components of a CRISPR/Cpf1 system.

In an aspect, a method of manipulating a DSB repair pathway in a cell during a genome engineering reaction is provided comprising introducing to the cell a polypeptide and/or polynucleotide disclosed herein.

In an aspect, a method of stimulating homology-directed repair of DSBs in a cell is provided comprising introducing to the cell a polypeptide and/or polynucleotide disclosed herein.

In an aspect, a method of inhibiting 53BP1 recruitment to DSB sites in a cell is provided comprising introducing to the cell a polypeptide and/or polynucleotide disclosed herein. The suppression of 53BP1 recruitment to DSB sites by the polypeptide may be monitored by methods known in the art, such as ionizing radiation focus formation.

In an aspect, a method of inhibiting 53BP1 recruitment to damaged chromatin in a cell is provided comprising introducing to the cell a polypeptide and/or polynucleotide disclosed herein.

In an aspect, a method of inhibiting 53BP1 function in a cell is provided comprising introducing to the cell a polypeptide and/or polynucleotide disclosed herein.

In an aspect, a method of increasing HR in a cell is provided comprising introducing to the cell a polypeptide, polynucleotide and/or expression vector disclosed herein. In certain embodiments, the method can further include introducing to the cell one or more components of a gene editing system as described herein. It should be understood that the cell can be any host cell as described herein.

In an aspect, a method of inducing DNA end resection in a cell is provided comprising introducing to the cell a polypeptide and/or polynucleotide disclosed herein.

In an aspect, a method of inducing BRCA1 recruitment to DSB sites in a cell is provided comprising introducing to the cell a polypeptide and/or polynucleotide disclosed herein.

In an aspect, a method of inhibiting 53BP1 function to DSB sites in a cell thereby increasing HR is provided comprising introducing to the cell a polypeptide and/or polynucleotide disclosed herein.

In an aspect, a method of inducing gene conversion in a cell is provided comprising introducing to the cell a polypeptide and/or polynucleotide disclosed herein.

In an aspect, a method of increasing the efficiency of HDR-mediated gene editing in a cell contacted with a CRISPR system is provided herein, comprising introducing to the cell a polypeptide and/or polynucleotide disclosed herein.

In an aspect, a method of increasing gene targeting in a cell is provided comprising introducing to the cell a polypeptide and/or polynucleotide disclosed herein in combination with an inhibitor of DNA-PK.

In an aspect, a method of modulating DNA end resection in a cell is provided comprising introducing to the cell a polypeptide and/or polynucleotide disclosed herein in combination with an inhibitor of DNA-PK.

In an aspect, the DNA-PK inhibitor is NU7441 [8-dibenzothiophen-4-yl-2-morpholin-4-yl-chromen-4-one, also known as KU-57788; Leahy J J, et al. Bioorg Med Chem Lett, 2004, 14 (24), 6083-608]; KU-0060648 (Munck J M, et al. Mol Cancer Ther, 2012, 11 (8), 1789-1798); NU7026 (Willmore E, et al. Blood, 2004, 103 (12), 4659-466); or PIK-75 (WO/2003/072557, Sep. 4, 2003).). In some embodiments, the DNA-PK inhibitor is AZD7648, M3814/nedesertib, CC-115 or BAY-8400.

In aspects, methods of the disclosure are used to treat a cell in G1 phase of the cell cycle (G1) or G0 phase of the cell cycle.

In aspects, a method of stimulating HR in a non-dividing cell is provided comprising introducing to the cell a polypeptide and/or polynucleotide disclosed herein.

In other aspects, methods of the present disclosure are administered to, or used to treat, a cell comprising engineered DSBs for genome modification or gene editing purposes.

The present disclosure also contemplates the use of methods, compositions and kits disclosed herein in genome modification, provided that said use is not a method for treatment of the human or animal body by surgery or therapy, and provided that said use is not a process for modifying the germ line genetic identity of human beings. Genome modification may comprise modifying a target polynucleotide sequence in a cell, modifying expression of a polynucleotide sequence in a cell, generating a model cell comprising a mutated disease gene, or knocking out a gene. A use of the present disclosure may further comprise repairing or editing a cleaved target polynucleotide by inserting an exogenous template polynucleotide, wherein the repair or editing results in a mutation comprising an insertion, deletion, or substitution of one or more nucleotides of the target polynucleotide.

Also contemplated herein is the use of methods, compositions and kits disclosed herein in genome engineering, provided that said use is not a method for treatment of the human or animal body by surgery or therapy, and provided that said use is not a process for modifying the germ line genetic identity of human beings. Genome engineering may comprise modifying a target polynucleotide sequence in a cell, modifying expression of a polynucleotide sequence in a cell, generating a model cell comprising a mutated disease gene, knocking in or knocking out a gene, or correcting one or more mutations in a gene locus. A use of the disclosure may further comprise repairing or editing a cleaved target polynucleotide by inserting an exogenous template polynucleotide, wherein said repair results in a mutation comprising an insertion, deletion, or substitution of one or more nucleotides of said target polynucleotide.

A method of homology directed repair in a cell of engineered DSBs for genome modification purposes is provided comprising introducing to the cell a polypeptide and/or polynucleotide disclosed herein.

The disclosure relates to the use of a polypeptide and/or polynucleotide disclosed herein in homology directed repair of engineered DSBs for genome modification purposes.

The disclosure relates to the use of a polypeptide and/or polynucleotide disclosed herein in homology-directed repair with single-stranded oligonucleotides (ssODNs).

The disclosure also relates to the use of a polypeptide and/or polynucleotide disclosed herein for stimulating homology-directed repair with single-stranded oligonucleotides (ssODNs).

In an aspect, a method disclosed herein for activating or stimulating HR in a cell further comprises a gene editing system. In an aspect the gene editing system comprises contacting the cell with a nuclease. Examples of nucleases include without limitation, zinc finger nucleases (ZFNs), engineered meganucleases, transcription activator like effector nucleases (TALENs), mega or homing endonucleases, clustered regularly interspaced short palindromic repeats (CRISPR)-associated (Cas) nucleases, Cpf1 nucleases, Ttago nucleases, and fusions between nucleases, such as mega-TALs and compact TALENs. In an aspect, the gene editing steps comprise the CRISPR/Cas9 system. In an aspect, the gene editing steps comprise the CRISPR/Cpf system.

A method of stimulating homology-based genome editing in a cell is provided comprising introducing to the cell a polypeptide disclosed herein.

In aspects, a gene editing system may correct a genomic modification. A genetic modification may comprise at least one mutation in a polynucleotide sequence having a locus associated with a genetic disorder. In an aspect, the genomic modification is selected from the group consisting of insertions, deletions and combinations thereof. In some embodiments, the genetic disorder is a monogenetic disorder. In some embodiments, the disorder is a multigenetic disorder. In some embodiments, the disorder is associated with one or more SNPs. In particular embodiments, the genomic modification corrects a point mutation.

In an aspect of a method of the disclosure to correct a genomic modification, the gene editing system comprises contacting the cell with a clustered regularly interspaced short palindromic repeats (CRISPR)-associated (Cas) protein and from one to two ribonucleic acids, wherein the ribonucleic acids direct Cas protein to, and hybridize to, a selected motif of a target polynucleotide sequence associated with a genetic disorder, wherein the target polynucleotide sequence is cleaved.

### Targeted Gene Insertion and Integration

The present disclosure provides compositions and methods for introducing an exogenous polynucleotide sequence into a target site of an endogenous polynucleotide sequence at a gene locus. In some embodiments, the polynucleotide sequence may comprise at least one mutation. In some embodiments, the mutation can cause aberrant expression and can manifest as a disease pathology. Methods to correct or ameliorate aberrant expression caused by a mutation associated with a disease state are described herein.

CRISPR-Cas systems are quickly emerging as an attractive tool to introduce double stranded breaks. Briefly, CRISPR-Cas systems utilize a guide RNA or guide polynucleotide to guide the Cas nuclease to a target site to introduce a double stranded break into the sequence.

A donor template or donor polynucleotide sequence can be used simultaneously to utilize HDR machinery that can resect the donor polynucleotide sequence into the endogenous sequence through the regions of the donor polynucleotide having high homology or sequence identity. In this manner, targeted gene insertion can be performed by administering a site-specific nuclease system in combination with a donor polynucleotide.

In embodiments, the donor polynucleotide comprises an exogenous sequence (including coding and non-coding regulatory sequences) that is flanked by regions containing high homology with the endogenous targeted locus. In some embodiments, the targeted gene insertion can replace at least a portion of the endogenous polynucleotide sequence. In particular embodiments, the exogenous sequence is integrated into the translational start site of the targeted gene locus. In particular embodiments, the exogenous sequence that is integrated into the host cell genome is expressed under control of the native promoter sequence of the targeted gene locus.

Endogenous polynucleotides may contain polymorphisms or mutations that cause expression of an aberrant protein that results in the manifestation of a disease. In some embodiments, the endogenous polynucleotide sequence comprises mutations, including but are not limited to missense and non-sense mutations. In some embodiments, the endogenous polynucleotide sequence can comprise insertions, deletions, or truncations.

### Donor Polynucleotides

In any of the foregoing embodiments, the donor polynucleotide can be comprised in a viral vector, a plasmid, a single-stranded oligodeoxynucleotide (ssODN), or any other suitable vector. In certain embodiments, the donor polynucleotide is comprised in an adeno-associated viral (AAV) vector. In certain embodiments, the AAV vector is an AAV1, AAV2, AAV3, AAV4, AAV3, AAV5, AAV6, AAV7, AAV8, AAV9, AAV10, AAV11, or AAV12. In a preferred embodiment, the AAV vector is AAV6.

In any of the foregoing embodiments, the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

In certain embodiments, integration of the donor polynucleotide sequence into a host cell genome can correct a mutation in a cell that is associated with a disease. In certain embodiments, integration of the donor polynucleotide sequence can replace a mutant allele in the host cell with a wild-type allele.

### Homology Arms

In preferred embodiments, the 5′ and 3′ homology arms of the donor polynucleotide have at least 95% sequence identity, respectively, with a distinct region of the target gene locus, so that HDR of the exogenous polynucleotide occurs only through the 5′ and 3′ homology arms, and the entirety of the exogenous polynucleotide sequence between the homology arms is integrated into the targeted locus. In some embodiments, the homology arms comprise sequences that target integration of the donor polynucleotide just downstream of the native promoter of the target gene, such that the integrated donor sequence is transcribed from and regulated by the native promoter sequence of the targeted gene. In other embodiments, the homology arms comprise sequences that target integration of the donor polynucleotide into the gene locus such that the target gene is replaced in whole or in part, for example, only with respect to regions of the target gene that harbor mutations. In some such embodiments, the target gene promoter is left intact in order to regulate expression of the transgene.

The homology arms can be of variable lengths. In some embodiments, the 5′ and 3′ homology arms can be identical in length. In some embodiments the 5′ and 3′ homology arms can be different lengths.

In some embodiments, the 5′ homology arm comprises about 50 base pairs to about 1,000 base pairs. In some embodiments, the 5′ homology arm comprises at least about 50 base pairs. In some embodiments, the 5′ homology arm comprises at most about 1,000 base pairs. In some embodiments, the 5′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

In some embodiments, the 3′ homology arm comprises about 50 base pairs to about 1,000 base pairs. In some embodiments, the 3′ homology arm comprises at least about 50 base pairs. In some embodiments, the 3′ homology arm comprises at most about 1,000 base pairs. In some embodiments, the 3′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

### CRISPR Systems

In some embodiments, a CRISPR enzyme in combination with (and optionally complexed with) a guide sequence is delivered to the cell. In some embodiments, methods for introducing a protein component into a cell according to the present disclosure (e.g. Cas9/gRNA RNPs) may be via physical delivery methods (e.g. electroporation, particle gun, Calcium Phosphate transfection, cell compression or squeezing), liposomes or nanoparticles. In some embodiments, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

Binding of the polynucleotide sequence recruits the Cas protein and facilitates a double-stranded break into the polynucleotide sequence by the Cas nuclease. In some embodiments, guide polynucleotide sequence binds to a region of a gene corresponding to the coding sequence. In some embodiments, the coding sequence is an exon. In some embodiments, the guide polynucleotide can bind to a region of the gene corresponding to a non-coding region. In some embodiments, the non-coding region is an intron or untranslated region (UTR).

In some embodiments, guide polynucleotide sequence comprises a chemical modification. In some embodiments, the guide polynucleotide sequence comprises a 2′-O-methyl-3′-phosphorothioate modification. Examples of chemical modifications to guide polynucleotide sequences which enhance stability and cleavage efficiency of CRISPR-Cas systems include but are not limited to those described in PCT Publication Nos. WO/2016164356 and WO 2016/089433, each of which is herein incorporated by reference in its entirety.

### Delivery Vectors

Donor polynucleotide sequences described herein may be incorporated within a wide variety of gene therapy constructs, e.g., to deliver a nucleic acid encoding a protein to a subject in need thereof. A vector construct refers to a polynucleotide molecule including all or a portion of a viral genome and an exogenous polynucleotide sequence. In some instances, gene transfer can be mediated by a DNA viral vector, such as an adenovirus (Ad) or adeno-associated virus (AAV). Other vectors useful in methods of gene therapy are known in the art. For example, a construct of the present invention can include an alphavirus, herpesvirus, retrovirus, lentivirus, or vaccinia virus.

Adenoviruses are a relatively well characterized group of viruses, including over 50 serotypes. Adenoviruses are tractable through the application of techniques of molecular biology and may not require integration into the host cell genome. Recombinant Ad-derived vectors, including vectors that reduce the potential for recombination and generation of wild-type virus, have been constructed. Wild-type AAV has high infectivity and is capable of integrating into a host genome with a high degree of specificity.

AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally.

At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure.

A serotype of a viral vector used in certain embodiments of the invention can be selected from the group consisting from AAV1, AAV2, AAV3, AAV4, AAV5, AAV6, AAV7, AAV8, and AAV9. Other serotypes are known in the art or described herein and are also applicable to the present disclosure. In particular instances, the present invention includes an AAV9 viral vector including a glucocerebrosidase nucleic acid of the present invention.

### Methods of Treatment

In some embodiments, the polypeptides or compositions of the present disclosure can be used to treat a disease by introducing the polypeptides to a cell targeted for HDR-mediated gene editing. In certain aspects, the method is not a process for modifying the germ line genetic identity of human beings. By way of example, but not limitation, the disease can be selected from the group consisting of cancer, cardiovascular diseases including heart failure, hypertension and atherosclerosis, respiratory diseases, renal diseases, gastrointestinal diseases including inflammatory bowel diseases such as Crohn's disease and ulcerative colitis, hepatic, gallbladder and bile duct diseases, including hepatitis and cirrhosis, hematologic diseases, metabolic diseases, endocrine and reproductive diseases, including diabetes, bone and bone mineral metabolism diseases, immune system diseases including autoimmune diseases such as rheumatoid arthritis, lupus erythematosus, and other autoimmune diseases, musculoskeletal and connective tissue diseases, including arthritis, achondroplasia, infectious diseases and neurological diseases such as Alzheimer's disease, Huntington's disease and Parkinson's disease.

Embodiments of the disclosure provide for treatment of various cancers including but not limited to carcinomas, melanomas, lymphomas, sarcomas, blastomas, leukemias, myelomas, osteosarcomas, neural tumors, and cancer of organs such as the breast, ovary, and prostate.

In embodiments, treatment of cancer with BRCA-1 defects, BRCA-2 defects, dual BRCA-1/BRCA-2 defects, and Fanconi anemia is provided. In some embodiments, the cancer is breast cancer, in particular invasive ductal carcinoma and invasive lobular carcinoma. In some embodiments, the cancer is ovarian cancer, in particular epithelial ovarian tumors, germ cell ovarian tumors, and sex cord stromal tumors.

Methods disclosed herein for activating homologous recombination may be used to genetically modify polynucleotides associated with a genetic disorder. In some embodiments, the genetic disorder is a monogenetic disorder. In some embodiments, the genetic disorder is a multigenetic disorder. In some embodiments, the genetic disorder is associated with one or more SNPs. In particular embodiments, the genomic modification corrects a point mutation.

Examples of genetic disorders and polynucleotide sequences associated with the genetic disorders may be found on the World Wide Web (see for example, the National Center for Biotechnology Information, National Library of Medicine (Bethesda, Mass.) or the McKusick-Nathans Institute of Genetic Medicine, Johns Hopkins University (Baltimore, Md.), listed in published patents and applications (see, for example, US Published Application No. 2015/0247150), and in publications (see for example, Turitz Cox D. B. et al, Nature Medicine 21, 121-131, 2015; and O'Connor T. P. and R. G. Crystal, Nature Reviews/Genetics Volume 7, April 2006, pages 261-276 including Supplementary Information, and publications cited therein.)

In an aspect, the genetic disorder is a genetic disorder of muscle. In an aspect, the genetic disorder is myotonic dystrophy type 1. In an aspect, the genetic disorder is myotonic dystrophy type 2. In an aspect, the genetic disorder is Duchenne muscular dystrophy (DMD). In an aspect, the genetic disorder is Becker muscular dystrophy.

In an aspect, the genetic disorder is a genetic disorder of the liver, for example, alpha-1 antitrypsin deficiency, Wilson Disease, hereditary hemochromatosis, Type I tyrosinemia, glycogen storage disease Type IV, argininosuccinate lyase deficiency, citrin deficiency, cholesterol ester storage disease and hereditary fructose intolerance.

In an aspect, the genetic disorder is alpha-1 antitrypsin deficiency which is an autosomal recessive (codominant) disease due to mutations in the SERPINA1 gene that encodes the serine protease inhibitor AAT.

In an aspect, the genetic disorder is Wilson disease which depends on mutations in the gene encoding the ATP7B Cu translocase, a protein mainly expressed by the hepatocyte that regulates the levels of copper in the liver.

In an aspect, the genetic disorder is a genetic disorder of the lungs.

In an aspect, the genetic disorder is cystic fibrosis, an autosomal recessive disease caused by mutations of the Cystic Fibrosis Transmembrane Regulator (CFTR) protein, a member of the ATP-binding cassette superfamily of transmembrane proteins.

In other aspects, the genetic disorder may be heamophilia, al-antitrypsin deficiency, Canavan disease, Adenosine deaminase deficiency, X-linked severe combined immunodeficiency, familial amyloidotic polyneuropathy, thalassemia, Tay-Sachs disease, late infantile ceroid lipofuscinosis, mucopolysaccharidosis, Niemann-Pick disease, achondroplasia, Huntington disease, spino-cerebellar ataxia, Fredriech ataxia, Amyotrophic Lateral Sclerosis, monogenic hypercholesterolemia and other monogenic disorders.

In aspects, the genetic disorder is sickle cell anemia and a method disclosed herein comprises correcting the mutated HBB hemoglobin gene by gene conversion with its paralog HBD.

In some embodiments, the gene edited cell is a hematopoietic stem or progenitor cell. Genetically modified hematopoietic stem or progenitor cells generated with the compositions and methods provided herein may be used as part of a treatment regimen for any disease or condition for which HSC transplantation (HSCT) is useful. HSCT may be used to treat a number of conditions, including congenital and acquired conditions. In some embodiments, acquired conditions treatable with HSCT include but are not limited to: (1) malignancies, including hematological malignancies such as leukemias (e.g. acute lymphoblastic leukemia (ALL), acute myeloid leukemia (AML), chronic lymphocytic leukemia (CLL), chronic myelogenous leukemia (CML)), lymphomas (e.g. Hodgkin's disease, Non-Hodgkin's lymphoma), myelomas (e.g. multiple myeloma (Kahler's disease)); solid tumor cancers (e.g. neuroblastoma, desmoplastic small round cell tumor, Ewing's sarcoma, choriocarcinoma); (2) hematologic disease, including phagocyte disorders (e.g. chronic granulomatous disease), bone marrow failure disorders (e.g. myelodysplastic syndrome, Fanconi's anemia, dyskeratosis congenita), anemias (e.g. paroxysmal nocturnal hemoglobinuria, aplastic anemia, acquired pure red cell aplasia), myeloproliferative disorders (e.g. polycythemia vera, essential thrombocytosis, myelofibrosis); (3) metabolic disorders including amyloidosis (e.g. amyloid light chain (AL) amyloidosis); (4) environmentally-induced diseases such as radiation poisoning; (5) viral diseases (e.g. HTLV, HIV); and (5) autoimmune diseases such as multiple sclerosis.

In some embodiments, congenital conditions treatable with HSCT include but are not limited to: (1) lysosomal storage disorders, including lipidoses (disorders of lipid storage, such as neuronal ceroid lipofuscinoses (e.g. infantile neuronal ceroid lipofuscinosis (INCL, Santavuori disease) and Jansky-Bielschowsky disease (late infantile neuronal ceroid lipofuscinosis)); sphingolipidoses (e.g. Niemann-Pick disease and Gaucher disease), leukodystrophies (e.g. adrenoleukodystrophy, metachromatic leukodystrophy, Krabbe disease (globoid cell leukodystrophy); mucopolysaccharidoses (e.g. Hurler syndrome (MPS I H, α-L-iduronidase deficiency), Scheie syndrome (MPS I S), Hurler-Scheie syndrome (MPS I H-S), Hunter syndrome (MPS II, iduronidase sulfate deficiency), Sanfilippo syndrome (MPS III), Morquio syndrome (MPS IV), Maroteaux-Lamy syndrome (MPS VI), Sly syndrome (MPS VII)); glycoproteinoses (e.g. Mucolipidosis II (I-cell disease), fucosidosis, aspartylglucosaminuria, alpha-mannosidosis); and Wolman disease (acid lipase deficiency); (2) immunodeficiencies, including T-cell deficiencies (e.g. ataxia-telangiectasia and DiGeorge syndrome), combined T- and B-cell deficiencies (e.g. severe combined immunodeficiency (SCID), all types), well-defined syndromes (e.g. Wiskott-Aldrich syndrome), phagocyte disorders (e.g. Kostmann syndrome, Shwachman-Diamond syndrome), immune dysregulation diseases (e.g. Griscelli syndrome, type II), innate immune deficiencies (e.g. NF-Kappa-B Essential Modulator (NEMO) deficiency (Inhibitor of Kappa Light Polypeptide Gene Enhancer in B Cells Gamma Kinase deficiency)); (3) hematologic diseases, including hemoglobinopathies (e.g. sickle cell disease, thalassemia (e.g. β thalassemia)), anemias (e.g. aplastic anemia such as Diamond-Blackfan anemia and Fanconi anemia), cytopenias (e.g. Amegakaryocytic thrombocytopenia) and hemophagocytic syndromes (e.g. hemophagocytic lymphohistiocytosis (HLH)).

In some embodiments, the disease or condition is selected from the group consisting of a hemoglobinopathy, a viral infection, X-linked severe combined immune deficiency, Fanconi anemia, hemophilia, neoplasia, cancer, amyotrophic lateral sclerosis, alpha antitrypsin deficiency, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. In some embodiments, the hemoglobinopathy is selected from the group consisting of sickle cell disease, α-thalassemia, β-thalassemia, and δ-thalassemia.

In an aspect, the target polynucleotide sequence is associated with a genetic disorder of the lung. In an embodiment the target polynucleotide sequence is associated with cystic fibrosis, in particular the polynucleotide sequence is the cystic fibrosis transmembrane conductor receptor (CFTR) locus. Mutations in the CFTR (e.g., deletion of phenylalanine at position 508 in exon 11) cause cystic fibrosis.

In an aspect, the target polynucleotide sequence is associated with a genetic disorder of muscle. In an aspect, the target polynucleotide sequence is associated with muscular dystrophies. In an aspect, the target polynucleotide sequence is associated with Duchenne muscular dystrophy (DMD) (mutations in the dystrophin gene). In an aspect, the target polynucleotide sequence is associated with Becker muscular dystrophy (mutations in the dystrophin gene). In an aspect the target polynucleotide is associated with myotonic dystrophy type 1 (mutations in the DMPK gene) or myotonic dystrophy type 2 (mutations in the CNBP gene).

In an aspect, the target polynucleotide sequence is associated with sickle cell anemia (mutated HBB hemoglobin).

In aspects, the targeted polynucleotide sequence is associated with a genetic disorder of the liver. In an aspect, the target polynucleotide sequence is associated with alpha-1 antitrypsin deficiency (mutations in the SERPINA1 gene). In an aspect, the targeted polynucleotide sequence is associated with Wilson disease (mutations in the gene encoding the ATP7B Cu translocase).

In an aspect, the methods of the disclosure further comprise providing a functional protein with enhanced characteristics as compared to its naturally occurring counterpart, in particular a functional protein lacking or deficient in a subject, for example for treating genetic disorders. In embodiments, the methods comprise integrating a sequence encoding a functional protein in a cell in a subject in need thereof by sequential administration of a gene editing system and one or more transgene(s) encoding a non-naturally occurring protein with enhanced properties as compared to its naturally occurring counterpart. In other embodiments, the methods comprise administering to the subject a genetically modified cell expressing a functional version of one or more proteins aberrantly expressed in a subject. Thus, an isolated cell may be introduced into the subject (ex vivo cell therapy) or a cell may be modified when it is part of the subject (in vivo). In certain embodiments, transgene(s) are delivered using a viral vector, a non-viral vector and/or combinations thereof.

Components of the methods of the disclosure may be delivered by delivery systems known in the art, including without limitation viral based systems or non-viral based systems. (See for example, Sambrook et al, supra; Findeis, Mark A., editor, Nonviral vectors for gene therapy: methods and protocols (Totowa, N.J.: Humana Press, c2001); Rolland, Alain and Sullivan, Sean M., editors, Pharmaceutical gene delivery systems (New York: Marcel Decker, c2003); Rolland, Alain, editor, Advanced gene delivery: from concepts to pharmaceutical products (Amsterdam: Harwood Academic, c1999); K. Kataoka, Taira, K., Niidome, T. editors, Non-viral gene therapy: gene design and delivery (Tokyo; New York: Springer, c2005); and S. Lasic, Danilo D., Liposomes in gene delivery (Boca Raton, Fla.: CRC Press, 1997).)

Conventional viral based systems may comprise, for example, retroviral, lentivirus, adenoviral, adeno-associated, SV40, polyoma, papilloma, picornavirus, pox, helper-dependent adenoviral, and herpes simplex virus vectors for gene transfer. In an aspect the viral based system, is an adenoviral vector or adeno-associated viral vector. Suitable plasmid expression vectors can also be used. Examples of plasmid expression vectors include, without limitation, commercially available expression vectors from Novagen (e.g., pET vectors, Rosetta™ (D3), Origami™ (DE3)), New England Labs, Inc (e.g., pMAL™ vectors), Invitrogen Inc. (e.g., pAd/CMV/VS-DEST™, pAd-DEST™ vector, pLenti4/V5-DEST™) and Clontech (e.g., pAdeno X™ and pAd5F35).

Examples, of non-viral based systems include lipofection, nucleofection, electroporation, microinjection, sonoporation, biolistics, virosomes, liposomes, immunoliposomes, polycation or lipid: nucleic acid conjugates, naked DNA, mRNA, nanomaterial-based delivery, artificial virons and agent-enhanced uptake of DNA.

In an aspect, the component is a polypeptide disclosed herein and it is delivered into a cell by electroporation, sonoporation, microinjection, liposomal delivery or nanomaterial-based delivery.

In an aspect, the component is a polynucleotide disclosed herein and it is delivered into a cell using a vector such as an adenovirus vector, retrovirus vector, adeno-associated virus vector, lentiviral vector, herpes virus vector, SV 40 vector, polyoma virus vector, papilloma virus vector, picornavirus vector, pox virus vector, or a helper-dependent adenovirus vector.

In certain embodiments, a method of integrating an exogenous polynucleotide sequence into the genome of a cell is provided that can include introducing to the cell: (a) a nuclease capable of generating a double-strand break within a gene locus of the cell; (b) a donor polynucleotide; and (c) a 53BP1-inhibiting polypeptide as described herein, or a polynucleotide or expression vector thereof, where upon generation of a double-strand break within the gene locus by nuclease, the donor polynucleotide sequence is integrated into the gene locus by homology directed repair (HDR) to yield gene-edited cells. In some embodiments, the method is performed ex vivo. The nuclease, donor polynucleotide sequence and 53BP1-inhibiting polypeptide can be the 53BP1-inhibiting polypeptide of any of the foregoing embodiments. In such embodiments, the cell can be any host cell of the present disclosure. In some embodiments, the host cell is isolated from a donor, for example, a healthy donor or a donor suffering from a genetic disorder. In certain aspects, the gene-edited cells can be used for further cell therapy of a patient by administering the resulting gene-edited cells to a patient. In some embodiments, the administering comprises an autologous transplantation of a pharmaceutical composition comprising cells that are genetically modified in accordance with the methods described herein. In other embodiments, the administering comprises an allogeneic transplantation of a pharmaceutical composition comprising cells that are genetically modified in accordance with the methods described herein. As a non-limiting example, gene-edited cells can be administered to a patient suffering from sickle cell disease and can be edited to correct a mutation associated with sickle cell disease or to incorporate a wild-type allele. In certain aspects, the integration of the donor polynucleotide sequence into the host cell genome corrects a mutation in the cell that is associated with a disease. In certain aspects, the gene locus of the cell can include one or more mutations associated with a disease or encodes an aberrant protein. In certain embodiments, integration of the donor polynucleotide sequence replaces a mutant allele in the cell with a wild-type allele. The disease can be any disease as disclosed herein. Non-limited examples of the disease can include a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders. Preferably, the hemoglobinopathy is sickle cell disease, α-thalassemia, β-thalassemia, or δ-thalassemia.

### Kits

The disclosure further provides a kit for performing an assay or method disclosed herein.

In some embodiments, a kit is provided the comprises a polypeptide, polynucleotide or vector of any of the foregoing embodiments.

In some embodiments, a kit is provided that comprises a polypeptide, polynucleotide or vector of any of the foregoing embodiments, and one or more components of a gene editing system as described herein. In any of the foregoing embodiments, the kit further comprises an inhibitor of DNA-PKcs.

In any of the foregoing embodiments, the kit can further include activators of DNA end resection and activators of homologous recombination discussed herein.

### EXAMPLES

The following examples are put forth so as to provide those of ordinary skill in the art with a complete disclosure and description of how to make and use the methods and compositions of the invention and are not intended to limit the scope of what the inventors regard as their invention.

### Example 1: Development of a Functional Screen to Identify Improved Peptide Inhibitors of 53BP1

### Materials and Methods

### Molecular Cloning:

The construct for the lentiviral-based expression and screening of i53 mutants was cloned from a third-generation lentiviral plasmid (Lenti SFFV) purchased from Twist Biosciences (SEQ ID NO: 73). An empty vector was constructed to include BamHI and NsiI restriction enzyme cut sites upstream of a T2A-mCherry-WPRE cassette (to enable fluorescence-based monitoring of cells expressing the i53 variants). The sequences for i53 variants were either ordered as gene fragments from Twist Biosciences or IDT or amplified from previously constructed plasmids using primers designed to introduce the desired amino acid variation(s). Pooled NNK and combinatorial libraries were constructed using NNK primers (IDT) or oligo pools (Twist Biosciences), respectively. Combinatorial libraries were designed using one codon per amino acid (A=GCA, C=TGC, D=GAC, E=GAG, F=TTC, G=GGT, H=CAC, I=ATA, K=AAA, L=CTC, M=ATG, N=AAT, P=CCT, Q=CAG, R=CGG, S=AGC, T=ACC, V=GTC, W=TGG, Y=TAT). In certain library compositions, cysteine (C) and methionine (M) were excluded due to their inherent reactivity and susceptibility to oxidation (for any given two position combinatorial library, N=324-400). Variants and libraries were cloned into the digested empty vector at the BamHI/NsiI cut sites using standard Gibson assembly protocols.

### Cell Culture:

Lenti-X HEK293T cells (Takara Bio) were cultured in DMEM (1×)+GlutaMAX-I (Gibco) supplemented with 10% FBS (Sigma). K562 cells (ATCC) were cultured in RPMI (Gibco) supplemented with 10% FBS and 1× penicillin-streptomycin (Gibco). All cells were grown in a humidified 37° C. incubator with 5% CO₂ and were passaged every 3-5 d.

### Lentiviral Production:

Lenti-X HEK293T cells (Takara Bio) were seeded at a density of 4.5×10⁶ cells per 10 cm dish 18-24 h prior to transfection. The prepared cells were co-transfected using the TransIT®-Lenti transfection reagent (Mirus Bio) with MISSION® Genomics Lentivirus Packaging Mix (Mirus Bio) and lentiviral plasmids containing i53 variants/libraries of interest. The viral supernatant was collected 48 h after transfection, passed through a 0.45-μm filter (Cytiva), flash frozen, and stored until use at −80° C. Viral titers were measured by FACS in K562 cells and were typically ~0.5-1.5×10⁷ TU/mL.

### AAV Production:

The HBB-targeting AAV6 vectors HBB-SNP and HBB-UBC-GFP have been previously described^{1,2}. The HBB-SNP AAV6 was produced by Viralgen and the HBB-UBC-GFP AAV6 was produced by Vigene. Titers used for CD34+ HSPC editing experiments were determined using droplet digital PCR (ddPCR).

### CD34+ HSPCs Culture:

Human CD34+ HSPCs were cultured as previously described^{1,2}. CD34+ HSPCs were purchased from AllCells and were isolated from G-CSF-mobilized peripheral blood from healthy donors. CD34+ HSPCs were cultured at 2.5×10⁵-5×10⁵ cells/mL in StemSpan™-AOF (Stemcell) supplemented with stem cell factor (SCF) (100 ng/mL), thrombopoietin (TPO) (100 ng/ml), FLT3-ligand (100 ng/mL), IL-6 (100 ng/ml) (all Peprotech) and UM171 (35 nM) (Selleckchem). Cells were cultured at 37° C., 5% CO₂, and 5% O₂.

### Lentiviral Transduction of CD34+ HSPCs:

CD34+ HSPC cells were transduced using lentivirus at MOIs of 0.25-1 at day 1 post thaw. Cells were concentrated using centrifugation (180 g×7 min), counted, and added at a concentration of 4×10⁶ cells/mL to media containing lentivirus, cyclosporin A (5 μM, Sigma Aldrich) and Synperonic F108 (0.5 mg/mL, Sigma Aldrich). After 4 h of incubation, cells were spun down, washed once with media, and seeded into lentivirus-free media at a density of 3.5×10⁵ cells/mL.

### Genome Editing of CD34+ HSPCs:

Chemically-modified single guide RNAs (sgRNAs) used to edit CD34+ HSPCs at the HBB locus were purchased from Synthego. The sgRNA sequences were modified by adding 2′-O-methyl-3′-phosphorothioate at the three terminal nucleotides of the 5′ and 3′ ends. The target sequence for the HBB sgRNA is as follows: HBB: 5′CTTGCCCCACAGGGCAGTAA-3′ (SEQ ID NO: 74). Cas9 protein (SpyFi Cas9) was purchased from Aldevron. The RNPs were complexed at a Cas9:sgRNA molar ratio of 1:2.5 at 25° C. for 10-15 min prior to electroporation. Cells were collected, counted, and pelleted at 180 g×7 min. The cell pellets were resuspended in Maxcyte buffer with complexed RNPs and electroporated using a Maxcyte EXPERT ATx Nucleofector. After electroporation cells were plated at 3.5×10⁵ cells/mL in media supplemented with cytokines and the desired AAV donor added at 5.0×10²-2.5×10⁴ vector genomes/cell. 24 h after nucleofection, cells were spun down, washed once with media, and seeded into AAV-free media at a density of 3.5×10⁵ cells/mL.

### Screening of Pooled Libraries:

Lentiviral-based i53 variant libraries were transduced at an MOI of ~0.2-0.5 (aiming for ~30% transduction and a coverage of >500 cells per library member in mCherry+/GFP+ cell population for each replicate tested). Three days after transduction, cells were edited in triplicate or quadruplet at HBB as described above, using HBB-UBB-GFP donor AAV6 (MOI of 2.5×10⁴ vector genomes/cell). Three days post editing, cells were pelleted and resuspended in media with DAPI (Miltenyi Biotec). Single, live, mCherry+/GFP+ and mCherry+/GFP-cells were collected using a FACSAria cell sorter (Becton Dickinson); purity of populations was confirmed by post-sort purity checks. Post sort, genomic DNA was harvested from each sorted cell population using a Quick-DNA 96 Plus Kit (Zymo Research). The DNA concentration of each sample was measured using a Qubit 1×dsDNA BR assay kit (ThermoFisher).

### Next Generation Sequencing (NGS) of Pooled Libraries:

An amplicon sequencing workflow was designed to sequence and quantify i53 variants within starting and post selection pools. Primers and PCR conditions were optimized to specifically amplify the entire variant coding sequence from plasmids, lentiviral libraries, as well as genomic DNA carrying lentiviral vector insertions. After the initial amplification, the i53 amplicons undergo an additional PCR amplification to add sequencing adapters and sample indexes to enable sample multiplexing. The resulting sequencing libraries were then sequenced on an Illumina MiSeq instrument using paired end reads to cover the full length of the i53 coding sequence.

### NGS Analysis:

The sequencing results were analyzed to identify and quantify all mutations within the coding sequence of i53 pooled libraries. Tests using individual variants combined to give specific mutant allele frequencies demonstrated the sequencing and analysis workflows could correctly detect and quantify the different i53 versions. This approach was used to confirm sequence diversity in plasmid and lentiviral libraries prior to screening. For screening data analyses, a first quality control was performed on all runs to validate good values in number of mapped reads (>1e5 reads per sample), percent reads carried over from parent (from cloning), and overall distribution of sequences (no bias). After that, i53 allele frequencies in GFP+ and GFP-populations were normalized by parent abundance, and then a fold-change enrichment was calculated by dividing normalized frequencies in GFP+ cells by the normalized frequencies in GFP-cells. All datasets contained an internal control (NNK-generated parent sequence) that was used to perform a last quality control of datasets, excluding sequencing runs where internal control abundance was more than 10% different from that of parent carry over control.

Data processing and visualizations were generated using R (v4.1.2) and the ggplot2 package. Variants were ranked by fold change over parent and any variant for which average was over 1.0 was flagged as ‘Better than parent’. In addition, a paired Student's t-Test was performed for each variant versus control to generate statistical significance (cutoff 0.05).

### Results

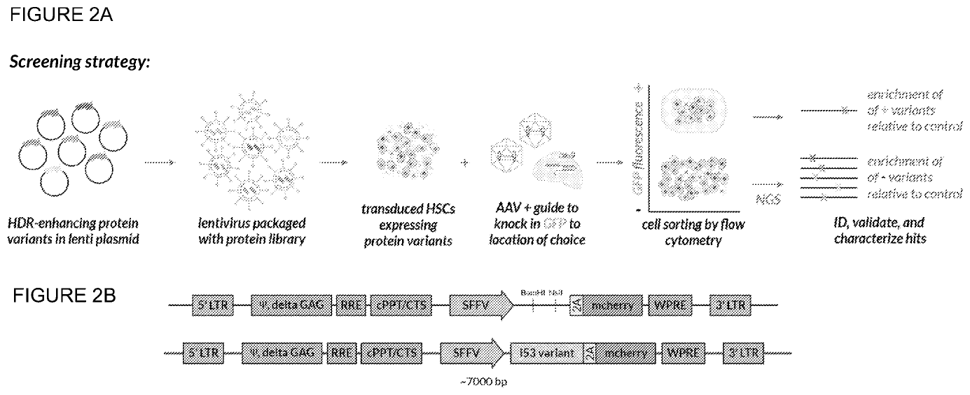

FIG. 2 provides a schematic outlining the development of a lentiviral-based pooled screening system in HSPCs used to identify HDR-enhancing proteins for a Cas9-mediated cut site of interest. As shown in FIG. 2A, protein variants were cloned into a lentiviral backbone, and the resulting plasmid library was then used to transfect Hek293 cells in order generate lentivirus packaged with the protein library of interest. Freshly thawed CD34+ HSPCs were then transduced with the library-encoding lentivirus at a level that leads to ~1 protein variant copy per transduced cell. Cas9, guide RNA, and an AAV6 template (encoding for a GFP insertion at the cut site of interest) were then used to edit the bulk transduced cell population. A few days post editing, the cells were sorted via flow cytometry into GFP+ and GFP-populations. Genomic DNA was extracted from each sorted cell population, sequenced via NGS, and analyzed to determine the distribution of variants relative to a control. Variants that were enriched in the GFP+ population relative to the control were then validated and characterized individually. As shown in FIG. 2B, in order to build out this screening system, a lentiviral-based construct was built to include restriction enzyme cut sites upstream of T2A-mCherry-WPRE cassette to easily clone in proteins of interest. Placement of T2A-mCherry tag at the 3′ end of the protein variants allowed for the independent expression of both the protein variant and mCherry post transcription and translation, enabling the fluorescence-based monitoring of cells that expressing the protein variants of interest.

The screening system was validated using previously characterized mutants of a known inhibitor of 53BP1, i534 i53 is an engineered ubiquitin variant identified through a binding-based screening and found to selectively bind of the Tudor domain of 53BP1. When transiently expressed in cells, i53 was found to block recruitment of 53BP1 to DSBs and improve HDR editing outcomes. The use of i53 as a protein-based additive was assessed for enhancement of HDR-mediated integration of a donor sequence, and mild increases (10-20%, using 1.5 mg/mL purified i53 in nucleofection solution) were observed with relatively inconsistent results (data not shown).

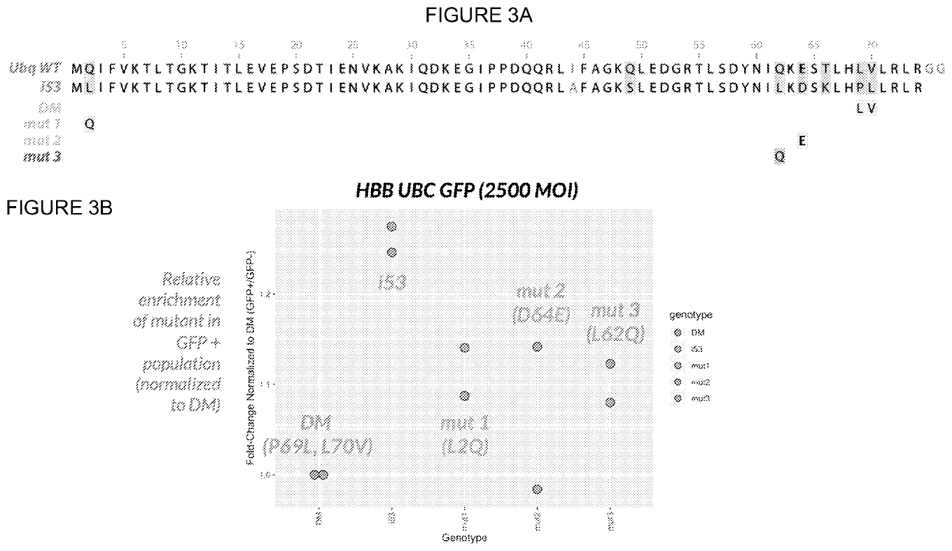

In order to validate the lentiviral-based screening system in HSPCs, different variants of i53 were cloned into the lentiviral vector described above: i53 (positive control), a previously reported dead mutant of i53 (“DM”=P67L L70V, negative control), and three mutants of i53 (“mut1”=L2Q, “mut2”=D64E, and “mut3”=L62Q) that were been previously reported to have decreased (but detectable) binding to the 53BP1 Tudor domain relative to i534 (FIG. 3A). After validating the differential effect these variants have on HDR when expressed via lentiviral transduction in HSPCs (data not shown), the plasmids encoding these five i53 variants were pooled together to generate a “mock” library. CD34+ HSPC cells were transduced using lentivirus packaged with the “mock” library and edited in duplicate at the HBB locus using HBB-UBC-GFP AAV (MOI=2500). As shown in FIG. 3B, NGS analysis of the gDNA purified from sorted mCherry+GFP+ and mCherry+GFP-populations indicated the expected differential variant enrichment in the mCherry+GFP+ population relative to the positive and negative controls (153 and DM).

### Example 2: Functional Screen Yields Improved 53BP1 Inhibitors that Boost HDR

### Materials and Methods

### Validation of Hits Via Lentiviral Expression:

Sequences of individual i53 variants of interest were cloned into the lentiviral-based expression plasmid described above. Hits were validated either as pooled “validation libraries” (variant and control plasmids manually mixed to generate a pool of 5-25 variants) or individually. Lentivirus generated from these plasmids was used to transduce CD34+ HSPC cells at MOIs of 0.5-1 at day 1 post thaw. At day 4, the transduced cells were edited with HBB-UBC-GFP AAV6 at concentrations of 1.25-2.5×10⁴ vector genomes/cell. Cells transduced with pooled validation libraries were edited in triplicate or quadruplet; cells transduced with individual variants were edited in duplicate.

For individual testing of variants, rates of integration of the HBB-UBC-GFP donor were measured using a Beckman Coulter CytoFLEX. DAPI (Miltenyi Biotec) was used to discriminate live and dead cells. mCherry expression was used to differentiate transduced cells from untransduced cells and rates of GFP integration were compared between the two populations to quantify the impact of lentiviral-based variant expression on HDR rates. Flow cytometry data were analyzed using FlowJo 10 software.

For pooled validation libraries, cells were sorted and analyzed as described above. NGS analysis of the gDNA purified from sorted mCherry+GFP+ and mCherry+GFP-populations was used to determine differential variant enrichment and validate the impact of individual variants on HDR rates relative to a control.

### Results

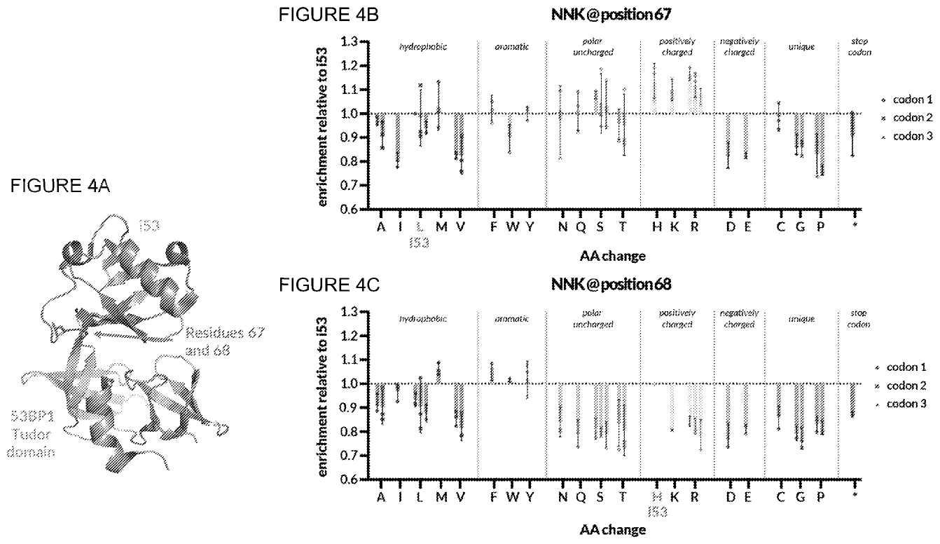

The validated lentiviral-based pooled functional screening system (described in Example 1) was used to identify single amino acid mutants of i53 that displayed enhanced HDR boosting capabilities relative to i53 at the HBB locus in HPSCs. A focused saturation mutagenesis library was generated using NNK primers in order to independently vary the amino acid identity of residues 67 and 68 of i53. As shown in FIG. 4A, these residues (shown in red) were targeted as sites for saturation mutagenesis due to their positioning at the i53/53BP1 Tudor domain binding interface (PDB=5JS6) 4. The side chain chemistry of these i53 surface residues was hypothesized to directly impact the interaction of i53 with the 53bp1 Tudor domain, potentially with a functional effect on HDR levels in cells. CD34+ HSPC cells were transduced using lentivirus packaged with this focused NNK library and edited in triplicate at the HBB locus using HBB-UBC-GFP AAV (MOI=1250). NGS analysis of the gDNA purified from sorted mCherry+GFP+ and mCherry+GFP-populations indicated differential variant enrichment in the mCherry+GFP+ population relative to i53. Consistent enrichment results were observed across codon duplications inherent in NNK libraries and across amino acid category at both residues 67 (FIG. 4B) and 68 (FIG. 4C). A positive charge at residue 67 seemed to be particularly beneficial to HDR levels at HBB relative to parent L67; the two top hits of the screen, L67R and L67H, were thus carried forward for additional validation and characterization.

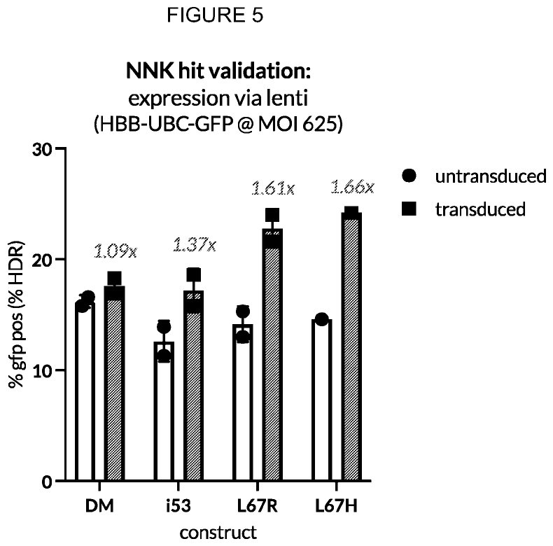

L67R and L67H i53 variants and controls (parental control i53 and negative control i53 dead mutant, “DM”=P67L L70V) were cloned individually into the lentiviral vector described above. CD34+ HSPC cells were transduced using lentivirus packaged with each construct and edited in duplicate at the HBB locus using HBB-UBC-GFP AAV (MOI=1250). Edited cells were analyzed via flow cytometry and the rates of GFP integration were compared between mCherry+ live cells (transduced, expressing the variant of interest) and mCherry-live cells (untransduced, control). As shown in FIG. 5, cell populations expressing the L67R and L67H i53 variants had higher rates of GFP integration in relation to the corresponding control cells than cells populations expressing i53 and DM. These results validate the observed differential enrichments of L67R and L67H relative to i53 in the pooled NNK screen.

### Example 3: Validation of Improved i53 Variants Comprising Modifications at Positions 67 and 68

### Materials and Methods

### i53 Variant Protein Production and Purification:

The sequences of different i53 variants were cloned into bacterial expression plasmids, resulting in a N-terminal His-tagged fusion protein with a protease cleavage site in between the 6×-His-tag and i53 variant sequence. The resulting plasmids were transformed into E. coli BL21 (DE3)-RIL for protein expression. Cells were grown at 30° C. in Luria-Bertani broth supplemented with 0.4% glucose to OD600=0.8 and induced with 0.4 mM IPTG at 16° C. for 18 hours. Cells were harvested by centrifugation, resuspended in 50 mM potassium phosphate pH 8.0, 500 mM NaCl, 20 mM imidazole, and 3 mM β-mercaptoethanol. Cells were lysed using an microfluidizer (Microfluidics). The crude lysate was immediately supplemented with 0.2 mM phenylmethylsulfonyl fluoride (PMSF) and centrifuged at 20,000 g for 20 minutes. The soluble fraction was subsequently incubated with 2 ml Ni-NTA (GE Healthcare) per 1,000 ODs for 1 hour at 4° C. Following incubation with the Ni-NTA resin, lysate was removed by pelleting the resin at 2,500 g for 3 minutes and washed 3 times with 9 bed volumes of 50 mM potassium phosphate pH 8.0, 500 mM NaCl, 20 mM imidazole, and 3 mM β-mercaptoethanol. Following the batch wash Ni-NTA resin was loaded onto a gravity column and His-tagged i53 mutant protein was eluted with 6 bed volumes of 50 mM potassium phosphate pH 8.0, 300 mM NaCl, 500 mM imidazole, and 3 mM β-mercaptoethanol. Eluted protein was dialyzed overnight against 10 mM Tris/HCl pH 8.0, 200 mM NaCl, and 1 mM DTT and the 6×His-tag was cleaved with protease. The protein was purified by anion exchange chromatography on a HiTrapQ column (GE Healthcare) via a linear NaCl gradient and twice by size exclusion chromatography using a Superdex S200 26/60 column (GE Healthcare) run in 10 mM Tris/HCl pH 8.0, 200 mM NaCl, 1 mM DTT. Proteins were concentrated to ~20 mg/mL and flash frozen for storage.

### Size Exclusion Chromatography: 53BP1 Tudor Domain: 153 Mutant Complex Identification

Recombinantly purified 53BP1 Tudor domain (53BP1 residues 1484-1603) was mixed with recombinantly purified i53 variants at a concentration of 0.5 mg/mL each. Proteins were incubated for 30 minutes at room temperature prior to injection onto an HPLC (Agilent, 1260 Infinity II). 5 μL of protein complex was injected onto a MAbPac 4×300 mm SEC column with 5 μm particle size and 300 Å pore size. The HPLC was run at 0.2 mL per minute using PBS as the mobile phase and continuously measuring the absorbance at 280 nm for approximately 1 full column volume. 53BP1 Tudor domain alone has a retention time of 14.6 min. i53 mutants have a retention time of ~15.5 min. A stable complex of 53BP1 Tudor domain: 153 mutants were found to have a retention time of 14.3 min.

### Bio-Layer Interferometry (BLI):

Data were collected using an Octet R8 system (Sartorius). Purified 53BP1 Tudor domain was labeled at exposed primary amine groups with NHS-biotin using ChromaLINK NHS-Biotin protein labeling kit (Vector Laboratories). 1 equivalent of chromalink biotin was incubated with the 53BP1 Tudor domain for 2 hours and buffer exchanged into fresh PBS. Labeling efficiency was calculated to be approximately 1 biotin per molecule of 53BP1 Tudor domain. Octet SA Biosensor tips (Sartorius) were incubated with biotin-labeled 53BP1 Tudor domain (ligand) for 60-80 seconds. The labeled tip was then dipped in 1× binding buffer (Sartorius) for 60 seconds to remove excess ligand and achieve baseline. Labeled tips were introduced to the i53 mutant (analyte) for 500-600 seconds and the response was continuously monitored to detect association. A range of analyte concentrations were tested from a highest to lowest concentration in nM (i.e. 200, 100, 50, 25, 12.5, 6.25, 3.125). The tips were then introduced to 1× binding buffer for 5 minutes and the response was continuously monitored to detect dissociation. A dissociation constant (K_{D}) was calculated using a 1:1 binding model and the on-rate (ka) and off-rate (ka) were calculated as a change in response (nm) over time (sec).

Editing CD34-HSPCs with i53 Variant Purified Proteins:

Purified i53 variant proteins were added to CD34+ HSPCs cells as part of the nucleofection mix at concentrations of 0.0125-1.6 mg/mL (volume of added protein≤ 1/10 of Maxcyte cuvette volume). CD34+ HPSCs were edited with protein variants 3 days post thaw and were harvested 2 days post nucleofection for NGS analysis or 3-5 days post nucleofection for GFP expression analysis. Editing reactions with proteins at a given concentration were repeated in duplicate or triplicate (either using the same CD34+ HSPC donor or across multiple donors).

When edited in combination with a DNAPK small molecule inhibitor (“DNAPKi,” AZD7648, Sellekchem #58832), cells nucleofected with i53 variant proteins were added to media containing both AAV and DNAPKi at a concentration of 0.5 μM. 24 h after nucleofection, cells were spun down, washed with media, and seeded into AAV and DNAPKi-free media at a density of 3.5×10⁵ cells/mL.

### Crystallography:

The human i53:53BP1 complex, purified in 10 mM Tris 8.0, 200 mM NaCl and 1 mM DTT was screened for crystallization at room temperature using a protein concentration of 30 mg/mL with the previously published condition⁴ 0.1 M MES (2-(N-morpholino) ethanesulfonic acid) pH 6.0, 0.2 M trimethylamine N-oxide and 25% (w/v) PEG MME (polyethylene glycol monomethyl ether) 2000. Crystals grew within 7 days at 23° C. using the sitting drop vapor diffusion method. Crystals were cryoprotected by adding glycerol, 20% (v/v) final concentration, to the reservoir solution before flash-freezing in liquid nitrogen. The i53:53BP1 complex was crystallized in the P2₁2₁2₁ space group with one i53:53BP1 complex molecule per asymmetric unit cell having dimensions of 40.3 Å×46.8 Å×89.7 Å.

### Structure Determination:

Structures of human i53:53BP1 Tudor domain (WT, L67H, L67R) were solved using molecular replacement and previously published structure of WTi53:53BP1 Tudor domain (PDB code: 5J26). The final models for human i53:53BP1 Tudor domain (WT, L67H, L67R) were built with native data and refined to an extended resolution of ~1.1 Å for each dataset. All models of i53:53BP1 complex were built using COOT⁵ and further refinement was completed.

### Measuring Targeted Integration of HBB-UBC-GFP (Flow Cytometry-Based Analysis):

Rates of targeted integration of the HBB UBC GFP donor were measured using a Beckman Coulter CytoFLEX. DAPI (Miltenyi Biotec) was used to discriminate live and dead cells. Flow cytometry data were analyzed using FlowJo 10 software.

### Measuring Targeted Integration of HBB-SNP (NGS-Based Analysis):

The frequency of homology directed repair (HDR) and other editing outcomes at HBB were measured using Next Generation Sequencing (NGS). An NGS assay was developed to determine the frequency of at the HBB locus by quantifying the total number of alleles that have been either: (1) not edited (% WT), (2) changed by HDR to incorporate sequence differences present in the AAV repair template (% HR), or (3) mutated during the genome correction process resulting in a gene that produces mutant β-globin (% INDELs).

For this assay, genomic DNA was harvested from cells using a Quick-DNA 96 Plus Kit (Zymo Research). The DNA concentration was measured using a Qubit 1×dsDNA BR assay kit (ThermoFisher). Purified genomic DNA was then used to amplify the HBB locus via polymerase chain reaction (PCR). The PCR products were diluted using nuclease-free water to serve as the template DNA for targeted NGS library prep. A second PCR with primers carrying partial Illumina adapters was performed to amplify a 142 base pair sequence that includes the region of the HBB locus that is to be corrected during the genome correction process. The PCR products were diluted again to serve as templates in a third PCR reaction using Nextera XT index primers. This third PCR reaction was used to assign unique identifiers to each sample and to add the full length adapter sequences necessary for Illumina sequencing. The size of the PCR products was assessed on an Agilent BioAnalyzer. PCR products were then pooled, purified using a Qiagen PCR purification kit, and quantified using PicoGreen in order to ready the PCR products for sequencing.

Based on the PicoGreen concentration, the library of pooled PCR products was diluted to a final concentration of 4 nM. Sequencing was performed on a MiSeq system using an Illumina MiSeq sequencing reagent kit (V2, 300 cycles). A 10% PhiX control library was added to the sample library to improve sequence diversity and to allow for error rate measurements. The library was denatured and loaded at 8-12 μM onto the sequencing reagent cartridge. The sequencing entails paired-end 150 base pair reads and dual indexing reads. The sequencing data was demultiplexed based on the sample indexes provided and FASTQ files for each sample are generated.

The FASTQ files were processed using the CRISPResso2 pipeline³. The data was reported as % WT, % corrected by HR, and % INDELs. The sequences containing INDELs were analyzed further to provide a summary of the sizes of the insertions and deletions created by the gene editing process. A subset of INDELs were classified as “HBD” based on sequence homology with the HBD gene (~93% sequence homology to HBB) and are thought to arise from homologous recombination with endogenous HBD. The remaining INDELs were classified as either NHEJ or MMEJ by leveraging previous experimental data (not shown) using a shRNA knockdown of POLQ (a key protein in MMEJ) and deducing the impact on individual INDEL profiles. INDELs affected by the POLQ knockdown (centering around a prominent-9 deletion) were classified as MMEJ; the remaining unaffected INDELs were classified as NHEJ.

A positive control with known editing outcomes, a negative control with no editing and a no template control were processed in parallel with each set of samples.

### Results

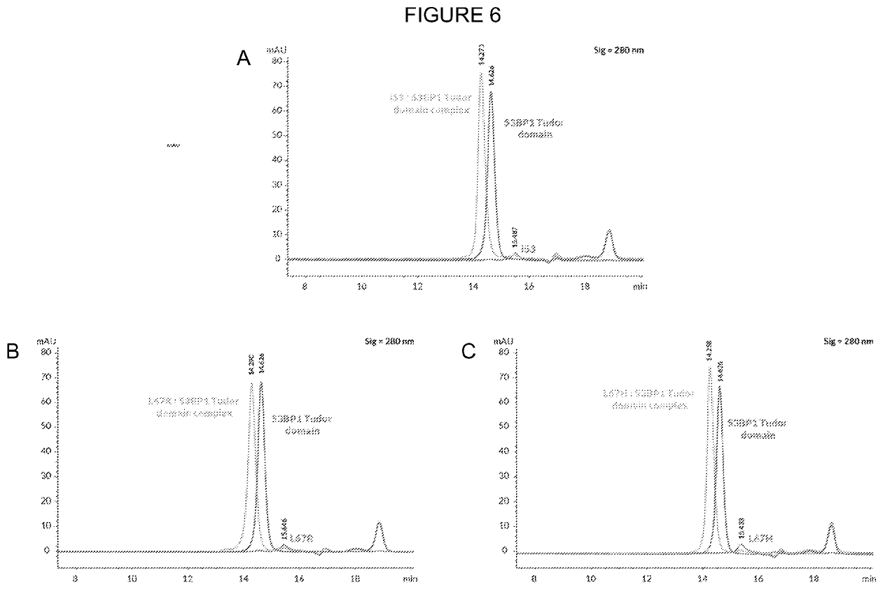

Size exclusion chromatography was performed to validate i53 variant binding with 53BP1 Tudor domain. i53 mutants and 53BP1 Tudor domain were run both individually and mixed on a size exclusion column (SEC) to monitor and assess complex formation and validate L67H and L67R as 53BP1 binders. As shown in FIG. 6, i53 variants alone have a retention time of approximately 15.5 min. The 53BP1 Tudor domain has a retention time of approximately 14.6 min. The two proteins mixed with a two-fold excess of i53 forms a complex and the retention time is shifted to approximately 14.3 min. FIG. 6A shows SEC traces of i53 alone, Tudor domain of 53BP1 alone, and mixed. FIG. 6B shows SEC traces of i53 variant L67R, Tudor domain of 53BP1, and mixed. FIG. 6C shows SEC traces of i53 variant L67H, Tudor domain of 53BP1, and mixed. i53, L67R, and L67H variants all appear to bind and similarly shift the 53BP1: i53 variant complex retention time, suggesting that L67R and L67H variant retain 53BP1 Tudor domain binding and thus likely increase levels of HDR through similar mechanisms of action as the i53 parent.

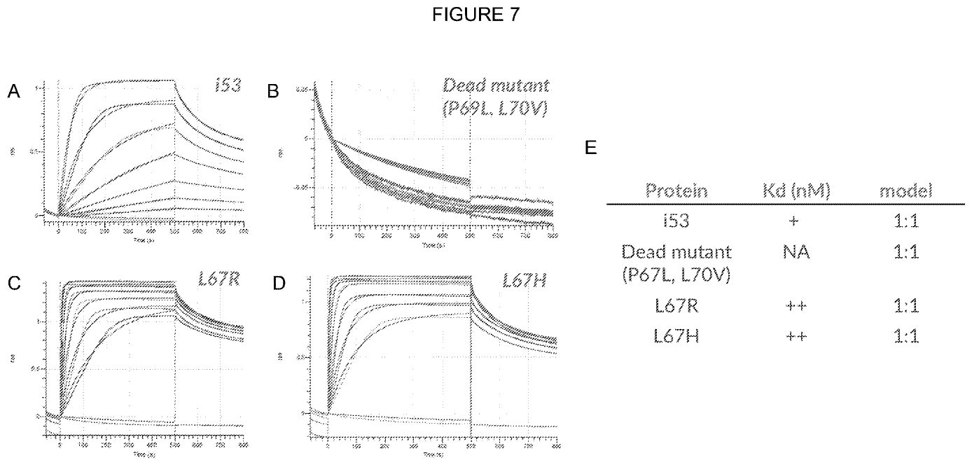

Biolayer Interferometry (BLI) was also performed to measure binding kinetics of i53 mutants with immobilized 53BP1 Tudor domain (FIG. 7). BLI was used to gather kinetic measurements (on- and off-rates) for i53 variant binding to biotin-labeled 53BP1 Tudor domain. The on- and off-rates measured at varying solution concentrations were used to calculate the dissociation constant (K_{D}) for each mutant listed. A 1:1 binding model was used for each K_{D} calculation. The + designation indicates a K_{D}>50 nM; ++ indicates a K_{D} between 5 and 50 nM. FIG. 7A shows i53 binding to immobilized 53BP1 Tudor domain. FIG. 7B shows dead mutant of i53 (“DM”, P69L L70V) binding to immobilized 53BP1 Tudor domain. FIG. 7C shows the mutant i53 L67R binding to immobilized 53BP1 Tudor domain. FIG. 7D shows the mutant i53 L67H binding to immobilized 53BP1 Tudor domain. FIG. 7E shows that both L67R and L67H have a measurably increased rate of binding compared to parent i53, likely contributing to the increased impact these proteins have on HDR levels relative to i53.

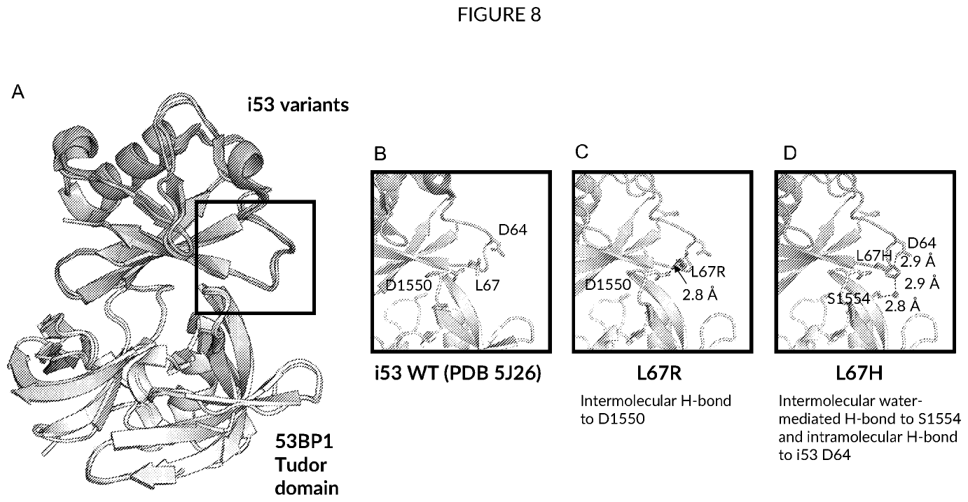

To further assess the binding interaction between the i53 variants L67R and L67H and 53bp1, x-ray crystallography was performed on structures of i53 variants complexed to the 53bp1 Tudor domain. FIG. 8A provides structural alignments of 53BP1 Tudor domain (yellow) bound to i53 variants WT (blue), L67R (green), and L67H (pink) with an RMSD <0.5 Å. A zoomed in view of the complex at the solvent-exposed loop preceding B5 for i53 (FIG. 8B), L67R (FIG. 8C), and L67H (FIG. 8D). The amino acid changes L67R and L67H of i53 were found to create new hydrogen bonds (denoted by black dotted lines): a 2.8 Å H-bond between L67R and D1550 of the 53BP1 Tudor domain, a 2.9 Å H-bond of L67H to D64 of i53, and a water mediated H-bond of L67H to S1554 of the 53BP1 Tudor domain. These new interactions likely contribute to the increased affinity of these two mutants to the 53BP1 Tudor domain.

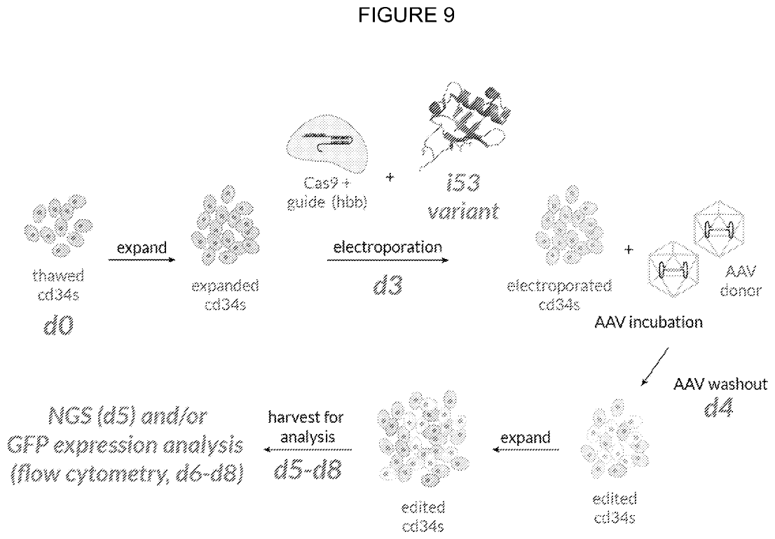

The L67R and L67H variants were then assessed as purified proteins for enhancement of HDR-mediated integration. FIG. 9 provides a schematic detailing the incorporation of purified i53 variants as HDR boosters into an HSPC gene editing workflow. Editing occurs 3 days post CD34+ HSPC cell thaw; purified i53 variants are incorporated at various concentrations into the nucleofection solution containing Cas9 and guide RNA. After nucleofection, the cells are incubated in media containing various concentrations of AAV for 24 h and harvested for analysis 24-96 hours post AAV washout.

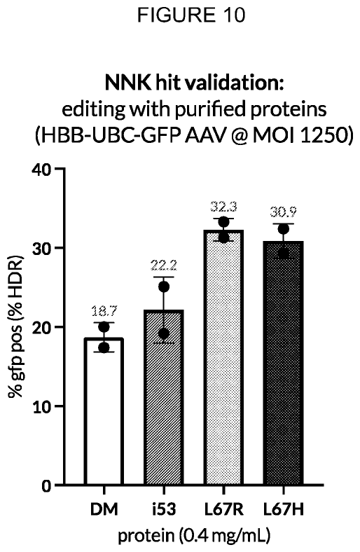

Editing of CD34+ HSPC cells was performed using HBB-UBC-GFP AAV as a donor template, in the absence and presence of the purified L67R and L67H i53 variants, as well as a parental control i53 and negative control i53 dead mutant, “DM”=P67L L70V. Purified proteins were incorporated into nucleofection solutions at a concentration of 0.4 mg/mL; post nucleofection, the cells were incubated with AAV at an MOI of 1250. Edited cells were analyzed via flow cytometry. As shown in FIG. 10, the use of L67R and L67H had a significant impact on GFP integration rates in live cells relative to parent i53. L67R and L67H were found to increase % HDR by an average of 69% over DM while i53 increased % HDR by only 19%. These results further validate the results of the pooled NNK screen (described in Examples 1 and 2), suggesting the functional screening platform can identify protein variants that can significantly boost HDR levels when used as protein-based additives for HDR-mediated gene editing.

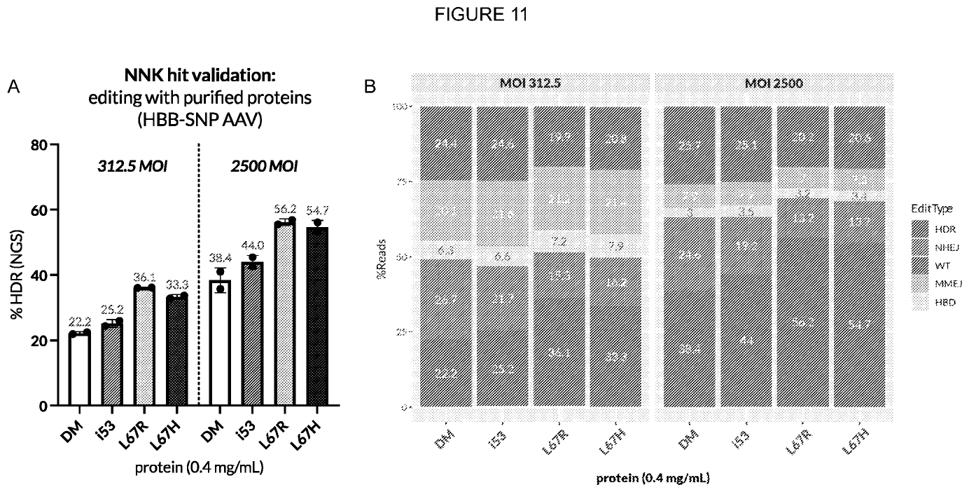

Editing of CD34+ HSPC cells was further assessed using the purified L67R and L67H i53 variants and HBB-SNP AAV, a donor template designed to correct the sequence encoding the E6V mutation in the HBB gene responsible for sickle cell disease^{1,2}. Protein variants (including a parental control i53 and negative control i53 dead mutant, “DM”=P67L L70V) were incorporated into nucleofection solutions at a concentration of 0.4 mg/mL; post nucleofection, the cells were incubated with AAV at MOIs of 312.5 and 2500. In order to quantify editing outcomes, gDNA was extracted from the edited cells and analyzed via NGS. As shown in FIG. 11A, L67R and L67H were found to have a significant impact on rates of HDR corrected alleles at both 312.5 and 2500 MOI relative to parent i53. L67R and L67H were found to increase levels of HDR corrected alleles by an average of 56% and 44% over DM when editing with MOIs of 312.5 and 2500, respectively. i53, however, only increased levels of % HDR corrected alleles by 14% and 15% when editing with MOIs of 312.5 and 2500, respectively. Notably, conditions incorporating L67R and L67H and MOI 312.5 resulted in levels of % HDR corrected alleles approaching those using 2500 MOI alone (a condition with 8× the amount of AAV). As shown in FIG. 11B, edited alleles from each sample were further characterized by type of edit (unedited WT, MMEJ, NHEJ, HBD, or HDR) and the resulting analyses are shown. The i53 variants were found to increase % HDR at the cost of % NHEJ (% MMEJ, and % HBD are unchanged with i53 variant treatment), with L67R and L67H having a more significant impact on % NHEJ relative to i53. These results align with the established mechanism of action of the i53 variants as well as the relative 53BP1 binding rates shown above.

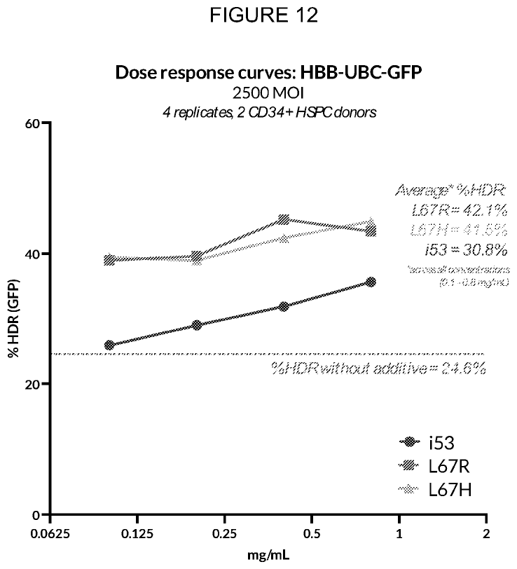

Dose response curves were then determined for purified i53 variants L67R and L67H relative to the parental control i53 using HBB-UBC-GFP AAV. Protein variants were incorporated into nucleofection solutions at concentrations of 0.1, 0.2, 0.4, and 0.8 mg/mL; post nucleofection, the cells were incubated with the AAV at an MOI of 2500. Edited cells were analyzed via flow cytometry. As shown in FIG. 12, cells treated with L67R and L67H maintained high levels of GFP integration across all editing conditions in live cells relative to the control (cells edited without a protein-based additive, average GFP integration across two replicates shown as grey dotted line) and i53 parent. L67R and L67H were found to increase levels of GFP integration by an average of 70% across all concentrations tested. The levels of GFP integration in cells treated with the highest concentration of i53 (0.8 mg/mL) were increased over the control edited cells by 45%; the effect of i53 on levels of GFP integration, however, decreased significantly as the protein concentrations decreased (29% at 0.4 mg/mL, 18% at 0.2 mg/mL, 5% at 0.1 mg/mL). These results suggest L67R and L67H are significantly more potent than parent i53 and align well with the relative 53BP1 binding rates shown above.

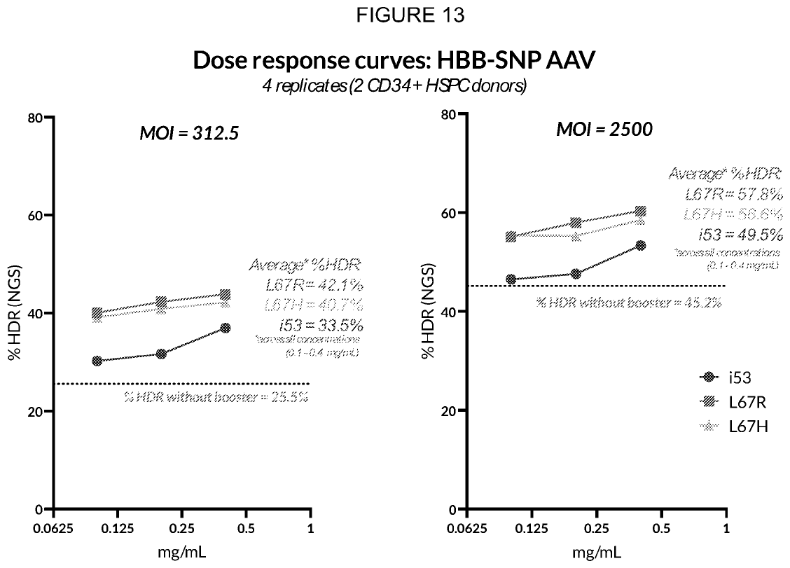

Similarly, dose response curves were determined for purified i53 variants L67R and L67H, relative to the parental control i53 using HBB-SNP AAV, using the corrective donor for the E6V sickle cell mutation^{1,2}. Protein variants were incorporated into nucleofection solutions at concentrations of 0.1, 0.2, and 0.4 mg/mL; post nucleofection, the cells were incubated with the AAV at an MOIs of 312.5 and 2500. In order to quantify editing outcomes, gDNA was extracted from the edited cells and analyzed via NGS. As shown in FIG. 13, cells treated with L67R and L67H maintained high levels of HDR corrected alleles across all editing conditions relative to the control (cells edited without a protein-based additive, average % HDR across four replicates shown as grey dotted line) and i53 parent. L67R and L67H were found to increase % HDR by an average of 62% and 27% using MOIs 312.5 and 2500, respectively, across all concentrations tested. As was previously observed, all conditions incorporating L67R and L67H and MOI 312.5 were found to result in levels of % HDR corrected alleles approaching those using 2500 MOI alone. The levels of HDR corrected alleles in cells treated with the highest concentration of i53 (0.4 mg/mL) were increased over the control edited cells by 45% and 18% at MOIs 312.5 and 2500, respectively. The effect of i53 on levels of % HDR, however, was found to decrease significantly as the protein concentrations decreased (312.5 MOI: 24% at 0.2 mg/mL and 18% at 0.1 mg/mL; 2500 MOI: 5% at 0.2 mg/mL and 3% at 0.1 mg/mL). These results confirm previous observations that L67R and L67H are significantly more potent than parent i53 and continue to align well with the relative 53BP1 binding rates shown above.

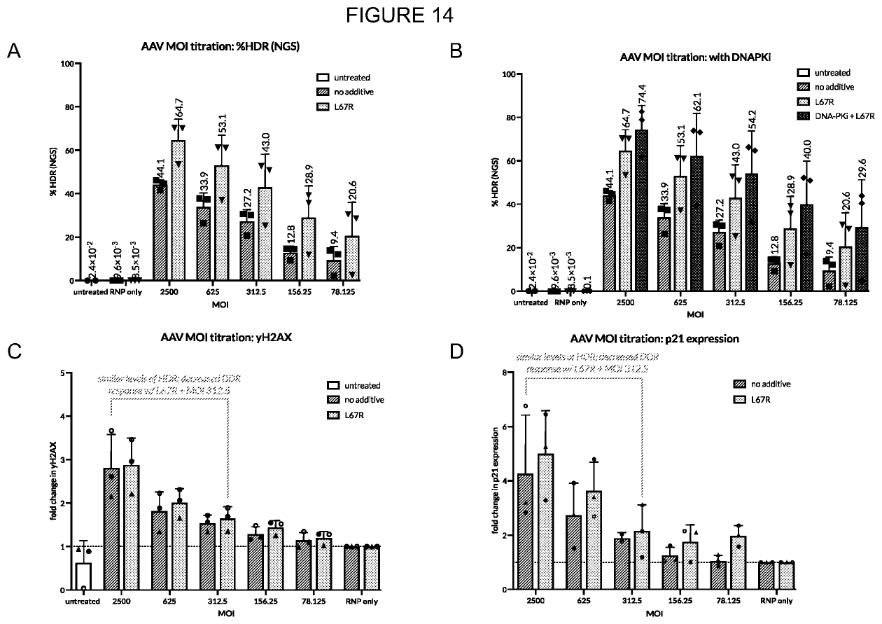

Editing of CD34+ HSPC cells was further assessed using an MOI titration of the HBB-SNP AAV donor (described above) and purified i53 variant L67R. L67R was incorporated into nucleofection solutions at of 0.8 mg/mL; post nucleofection, the cells were incubated with the AAV at range of MOIs (78.125-2500) with and without the addition of an additional NHEJ small molecule inhibitor DNAPKi (AZD7648, 0.5 M). In order to quantify the induction of the DNA damage response (DDR) pathways, a subset of edited cells was harvested 24 h post nucleofection to quantify DDR markers p21 and yH2AX⁵. At 48 h post nucleofection, DNA was extracted from the remaining edited cells and analyzed via NGS in order to quantify editing outcomes. As shown in FIG. 14A, cells treated with L67R maintained high levels of HDR corrected alleles across all editing conditions relative to the control (cells edited without a protein-based additive). The effect of using L67R on % HDR, was significantly higher with lower MOI conditions (156.25: 225%, 78.125: 220%) than with higher MOIs (2500: 47%, 625: 57%, 312.5: 58%). As shown in FIG. 14B, the addition of DNAPKi to media containing cells edited with L67R further increased the levels of HDR corrected alleles relative to the control (2500: 69%, 625: 83%, 312.5: 99%, 156.25: 313%, 78.125: 315%). These results suggest that L67R and other high affinity i53 variants can be used in combination with DNAPKi to drastically increase baseline editing rates by inhibiting NHEJ outcomes.

AAV concentration-dependent increases in yH2AX (FIG. 14C) and p21 (FIG. 14C) expression were identified through the quantification of these DDR markers in edited HSPCs. The addition of L67R was not found to significantly increase the yH2AX and p21 response using any given MOI. These results suggest that L67R and other high affinity i53 variants can be leveraged to maintain high levels of editing while enabling a beneficial decrease in AAV6.

### Example 4: Functional Screen of Combinatorial Library Yields Additional Improved HDR Boosters

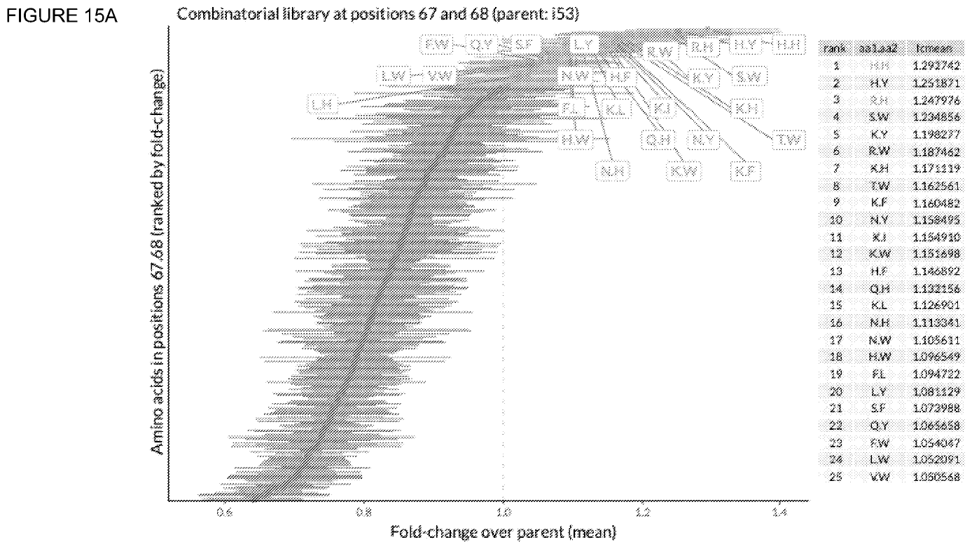

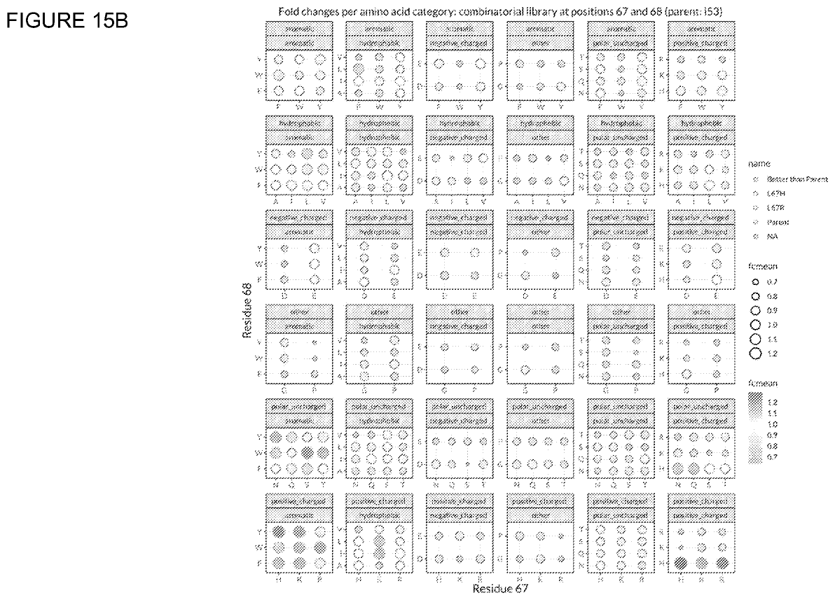

The validated lentiviral-based pooled functional screening system was further leveraged to identify additional HDR boosting mutants by screening combinatorial libraries targeting residues at the 53bp1/i53 interface. A combinatorial library was designed to explore all amino acid combinations at the previously screened residues L67 and H68. CD34+ HSPC cells were transduced using lentivirus packaged with this combinatorial library and edited in quadruplet at the HBB locus using HBB-UBC-GFP AAV (MOI=2500). As shown in FIG. 15A, NGS analysis of the gDNA purified from sorted mCherry+GFP+ and mCherry+GFP-populations indicated differential variant enrichment in the mCherry+GFP+ population relative to i53 (cysteine, C, and methionine, M, were excluded in order to avoid potential liabilities due to their inherent reactivity and susceptibility to oxidation). Hits that were significantly enriched relative to i53 (shown in purple) included L67H (shown in green) and L67R (shown in blue). When analyzing the results by amino acid categories, clear trends among hits emerged, as shown in FIG. 15B. In addition to the previously identified hits (L67R and L67H), combining positively charge and/or polar uncharged amino acids at residue 67 with aromatic amino acids at residue 68 appears to be particularly beneficial to HDR levels at HBB relative to parent L67 H68. A large subset of these variants (i.e. L67H H68Y, L67S H68W, etc) were thus carried forward for additional validation and characterization.

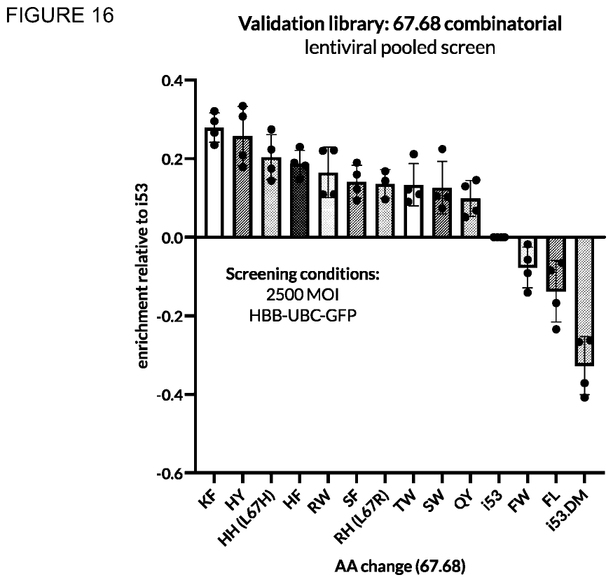

Top variants identified from the combinatorial screen above were cloned individually into the lentiviral vector shown above; the resulting plasmids were pooled together along with controls relative to (parental control i53 and negative control i53 dead mutant, “DM”=P67L L70V) to generate a small validation library. CD34+ HSPC cells were transduced using lentivirus packaged with this library and edited in quadruplet at the HBB locus using HBB-UBC-GFP AAV (MOI=2500). As shown in FIG. 16, NGS analysis of the gDNA purified from sorted mCherry+GFP+ and mCherry+GFP-populations confirmed positive enrichment for the majority of the double mutant variants tested relative to i53.

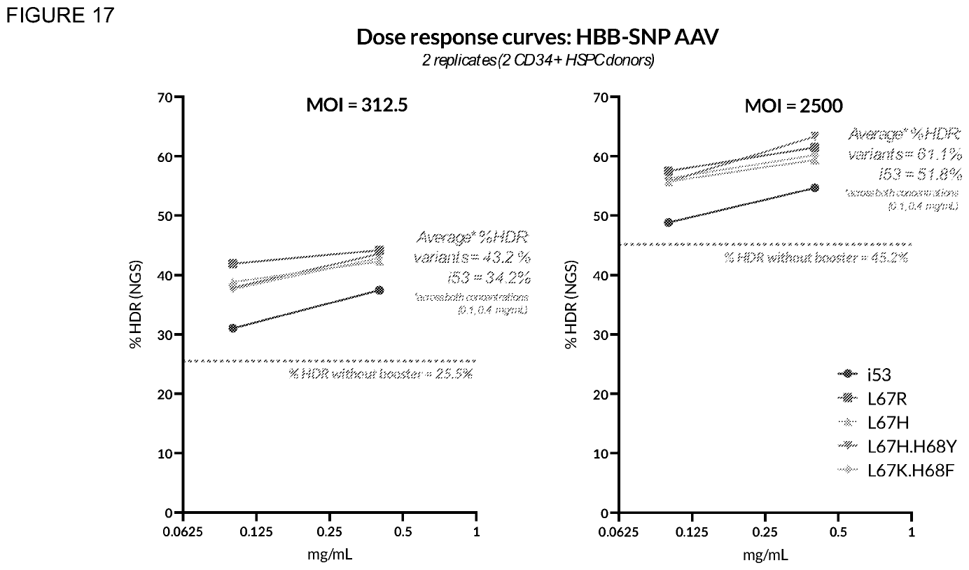

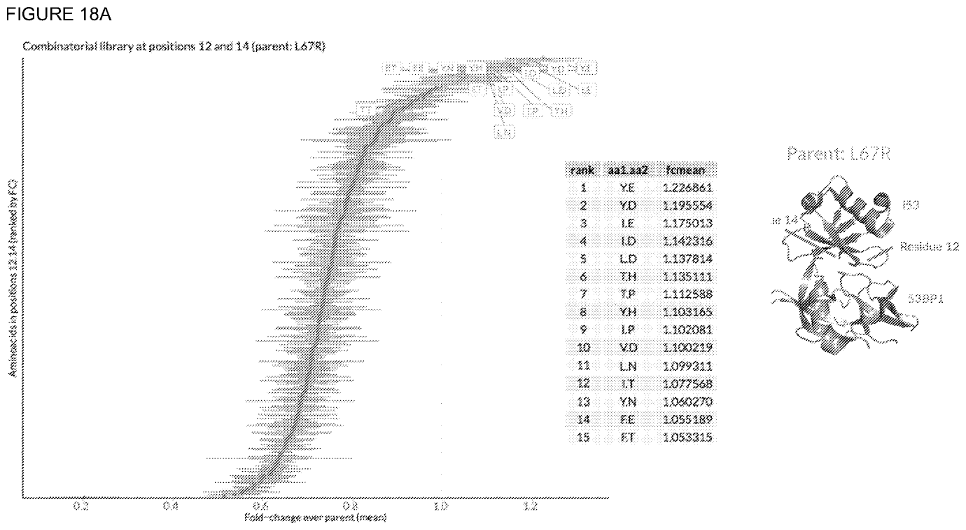

Editing of CD34+ HSPC cells was assessed using the HBB-SNP AAV donor (described above) and two representative purified double mutants of i53 (L67K H68F and L67H H68Y). Protein variants (including L67R, L67H, a parental control i53 and negative control i53 dead mutant, “DM”=P67L L70V) were incorporated into nucleofection solutions at concentrations of 0.1 and 0.4 mg/mL; post nucleofection, the cells were incubated with the AAV at MOIs of 312.5 and 2500. gDNA was extracted from the edited cells and analyzed via NGS. As shown in FIG. 17, cells treated with both the double and single i53 mutants maintained high levels of HDR corrected alleles across all editing conditions relative to the control (cells edited without a protein-based additive, average % HDR across four replicates shown as grey dotted line) and i53 parent. The four i53 mutants tested were found to increase % HDR by an average of 70% and 35% using MOIs 312.5 and 2500, respectively, across all concentrations. Incorporation of i53 also increased % HDR relative to control, but at a significantly lower level than the i53 mutants (312.5 MOI: 47% at 0.4 mg/mL and 22% at 0.1 mg/mL; 2500 MOI: 22% at 0.4 mg/mL and 8% at 0.1 mg/mL). These results further validate the results of the combinatorial library and validation screen, again suggesting the functional screening platform (described in Examples 1 and 2) can identify protein variants that can significantly boost HDR levels when used as protein-based additives for HDR-mediated gene editing.

### Example 5: Additional Improved HDR Boosters Comprising Modifications at Position 67 and/or 68

### Materials and Methods

### AAV Production (HBA-UBC-GFP, CCR5-UBC-GFP, and IL2RG-UBC-GFP):

All AAV6 vectors were cloned into the pAAV-MCS plasmid (Agilent Technologies), which contains inverted terminal repeats (ITRs) derived from AAV2. Left and right homology arms (LHAs/RHAs) were derived from human genomic DNA to match the indicated length at the respective knock-in sites. The left and right homology arm lengths for the HBA, CCR5, and IL2RG donors were as follows: HBA LHA: 976 bp, HBA RHA: 879 bp, CCR5 LHA: 502 bp, CCR5 RHA: 500 bp, IL2RG LHA: 400 bp, IL2RG RHA: 414 bp. Each vector contained a UBC promoter, a CopGFP, and a BGH polyA; UBC-GFP-BGH was synthesized as a gene fragment (Twist Bioscience) and cloned into pAAV with the corresponding LHA and RHA using standard Gibson Assembly protocols. The assembled LHA-UBC-GFP-BGH-RHA sequences for HBA-UBC-GFP, CCR5-UBC-GFP, and IL2RG-UBC-GFP AAV donors are provided herein as SEQ ID NO: 75, SEQ ID NO: 76 and SEQ ID NO:77, respectively.

The HBA-UBC-GFP AAV6 was produced by Packgene. CCR5-UBC-GFP AAV6 and IL2RG-UBC-GFP AAVG was produced by Vigene. Titers used for CD34+ HSPC editing experiments were determined using droplet digital PCR (ddPCR).

### Genome Editing of CD34+ HSPCs (HBA, CCR5, Il2RG):

Chemically-modified single guide RNAs (sgRNAs) were used to edit CD34+ HSPCs at the HBA, CCR5, and IL2RG loci. The sgRNA sequences were modified by adding 2′-O-methyl-3′-phosphorothioate at the three terminal nucleotides of the 5′ and 3′ ends. The target sequences for the HBA, CCR5, and IL2RG sgRNA are as follows: HBA: 5′-GGCAAGAAGCATGGCCACCG-3′ (SEQ ID NO: 78), CCR5: 5′-GCAGCATAGTGAGCCCAGAA-3′ (SEQ ID NO: 79) IL2RG: 5′-TGGTAATGATGGCTTCAACA-3′ (SEQ ID NO: 80). Cas9 protein (SpyFi Cas9) was purchased from Aldevron. The RNPs were complexed at a Cas9:sgRNA molar ratio of 1:2.5 at 25° C. for 10-15 min prior to electroporation. Cells were collected, counted, and pelleted at 180 g×7 min. The cell pellets were resuspended in Maxcyte buffer with complexed RNPs and electroporated using a Maxcyte EXPERT ATx Nucleofector. After electroporation cells were plated at 3.5×10⁵ cells/mL in media supplemented with cytokines and the desired AAV donor added at 5.0×10²-2.5×10⁴ vector genomes/cell. 24 h after nucleofection, cells were spun down, washed once with media, and seeded into AAV-free media at a density of 3.5×10⁵ cells/mL.

Purified i53 variant proteins were added to CD34+ HSPCs cells as part of the nucleofection mix at concentrations of 0.4 mg/mL (volume of added protein≤ 1/10 of Maxcyte cuvette volume). CD34+ HPSCs were edited with protein variants 3 days post thaw and were harvested 3-5 days post nucleofection for GFP expression analysis. Editing reactions with proteins at a given concentration were repeated in duplicate or triplicate (either using the same CD34+ HSPC donor or across multiple donors).

### Measuring Targeted Integration of HBA-UBC-GFP, CCR5-UBC-GFP, and IL2RG-UBC-GFP (Flow Cytometry-Based Analysis):

Rates of targeted integration of the HBA-UBC-GFP, CCR5-UBC-GFP, and IL2RG-UBC-GFP donors were measured using a Beckman Coulter CytoFLEX. DAPI (Miltenyi Biotec) was used to discriminate live and dead cells. Flow cytometry data were analyzed using FlowJo 10 software.

### Measuring Editing of OT-1 (NGS-Based Analysis):

Editing outcomes at the off-target (OT) editing site OT-1 can be assessed using an assay very similar to the one described above for measuring the targeted integration of HBB-SNP. The off-target editing site was identified via three different methods (in silico prediction,¹³ Circle-Seq,¹⁴ and Guide-Seq¹⁵) and was confirmed to be off-target editing site of significance via amplicon sequencing. The workflow for the OT-1 amplicon sequencing assay is very similar to the on-target sequencing assay but involves one less PCR step. A small 166 base pair sequence encompassing the OT-1 editing site is amplified directly from genomic DNA in the first PCR and then tagged with barcode and adapter sequences in a second PCR. The resulting PCR products are sized, quantified, and prepared for sequencing in the same manner as for the HBB on-target sequencing assay. The sequencing data is also processed using the Crispresso2 pipeline,³ but in contrast to the on-target sequencing assay no homology direct repair outcomes are present at OT-1, only % WT and % INDELs are reported.

### Poly-Reactivity Assay:

ELISA plates were coated separately with 50 μL of one of the following antigens diluted in 50 mM carbonate pH 9.0: cardiolipin (200 μg/mL, C0563; Sigma), KLH (20 μg/mL, H8283; Sigma), LPS (5 μg/mL, tlrl-eblps; InvivoGen), dsDNA (4 μg/mL, D4522; Sigma), insulin (20 μg/mL, 19278; Sigma) and RNA (4 μg/mL). 50 μL of purified 53BP1 Tudor domain was also coated on ELISA plates and diluted in PBS at (5 μg/mL). The coated plates were incubated overnight at 4° C. and blocked the following day with PBS+0.5% BSA at room temperature (RT) for 1 hour, followed by three washes with PBST (PBS plus 0.1% Tween 20). Fifty microliters of 100 nM anti-his hrp (1:2000 dilution in blocking buffer, 43360; Qiagen) was added to each well and incubated at RT for 1 hour. Plates were then washed with PBST and 100 μL of chemiluminescent imaging reagent (SuperSignal ELISA Pico Chemiluminescent Substrate, 37069; Thermo) was added to measure signal. Signal/background was calculated as follows:

$$\frac{{SignalSignal}_{coated well}}{{Signal}_{not coated well}} .$$

### Differential Scanning Fluorimetry and Dynamic Light Scattering:

For nanoDSF and nanoDLS measurements i53 variants were diluted to 0.5 mg/mL in 1×PBS pH 7.4 (cat #10010023, ThermoFisher Scientific) and evaluated on a Prometheus Panta (NanoTemper) using high sensitivity capillaries (cat #PR-C006, NanoTemper). Melting temperatures were calculated from a two state model between 25° C. and 95° C. using PR.PantaAnalysis (NanoTemper) and averaged from three technical replicates.

### Results

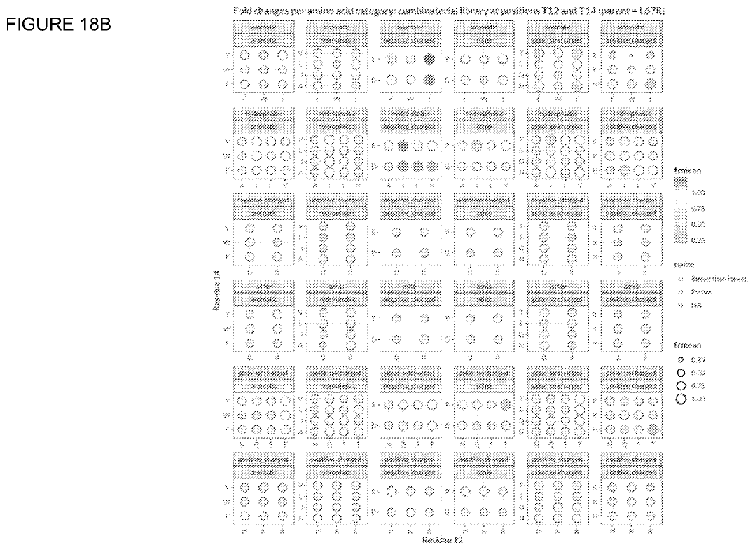

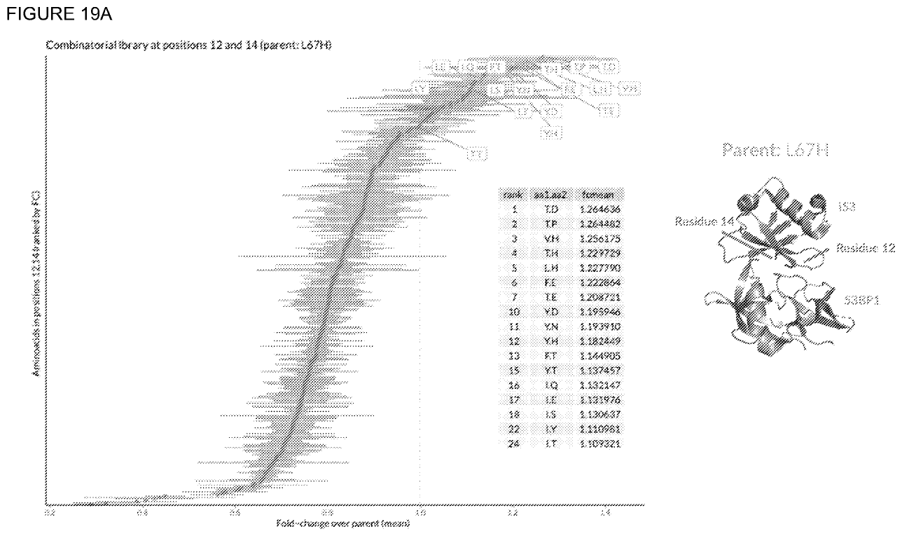

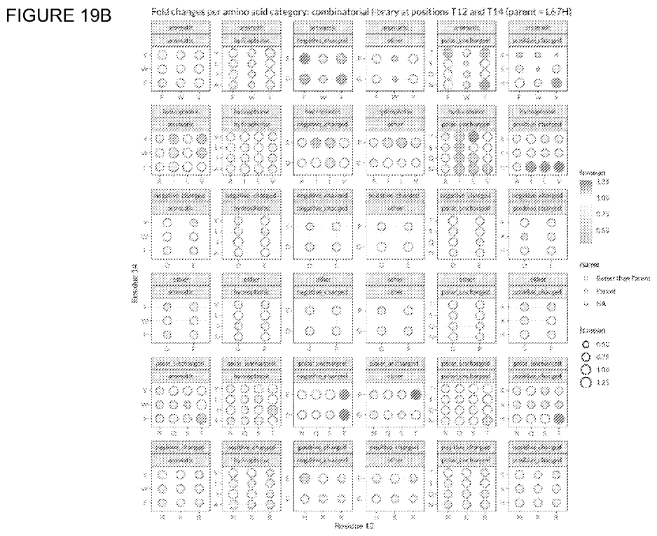

Additional HDR boosting mutants of i53 were identified by iterating on the L67R i53 variant and screening combinatorial libraries targeting additional residues at the 53bp1/i53 interface. A combinatorial library was designed to explore all amino acid combinations at positions 12 and 14 using L67R as the parent sequence. CD34+ HSPC cells were transduced using lentivirus packaged with this combinatorial library and edited in triplicate at the HBB locus using HBB-UBC-GFP AAV (MOI=2500). As shown in FIG. 18A, NGS analysis of the gDNA purified from sorted mCherry+GFP+ and mCherry+GFP-populations indicated differential variant enrichment in the mCherry+GFP+ population relative to parent L67R (T12 and T14, shown in blue). As shown in FIG. 18B, when analyzing the results by amino acid categories, clear trends among hits emerged; combining aromatic and/or hydrophobic amino acids at residue 12 with negative charge at residue 14 seemed to be particularly beneficial to HDR levels at HBB relative to parent T12 T14 L67R. A subset of these variants (i.e. T12Y.T14E.L67R and T12I.T14D.L67R etc) were thus carried forward for additional validation and characterization.

Additional HDR boosting mutants of i53 were identified by iterating on the L67H i53 variant and screening combinatorial libraries targeting additional residues at the 53bp1/i53 interface. A combinatorial library was designed to explore all amino acid combinations at positions 12 and 14 using L67R as the parent sequence. CD34+ HSPC cells were transduced using lentivirus packaged with this combinatorial library and edited in triplicate at the HBB locus using HBB-UBC-GFP AAV (MOI=2500). As shown in FIG. 19A, NGS analysis of the gDNA purified from sorted mCherry+GFP+ and mCherry+GFP-populations indicated differential variant enrichment in the mCherry+GFP+ population relative to parent L67H (T12 and T14, shown in blue). As shown in FIG. 19B, When analyzing the results by amino acid categories, similar trends among hits were observed as in the analogous library with parent L67R (FIG. 18, i.e. pairing aromatic and/or hydrophobic amino acids at residue 12 with negative charge at residue 14), but additional beneficial amino acid combinations at residues 12 and 14 emerged as hits relative to parent L67H (i.e. pairing hydrophobic amino acids at residue 12 with a histidine at residue 14 or substituting residue 14 with a negatively charged amino acid while retaining the parental threonine at residue 12). A subset of these variants (i.e. T12V.T14H.L67H and T14D.L67H etc) were thus carried forward for additional validation and characterization.

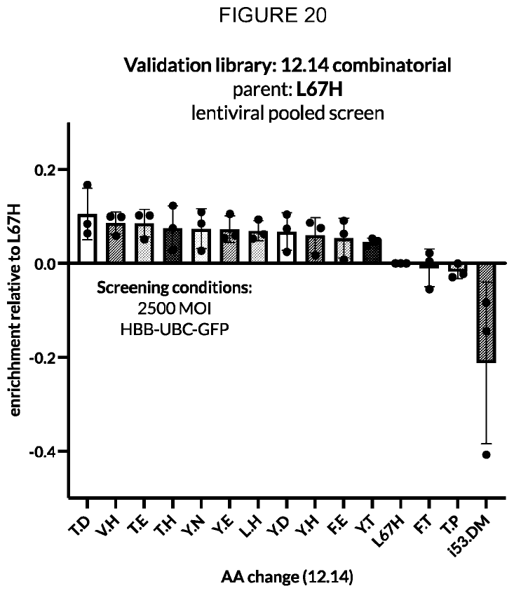

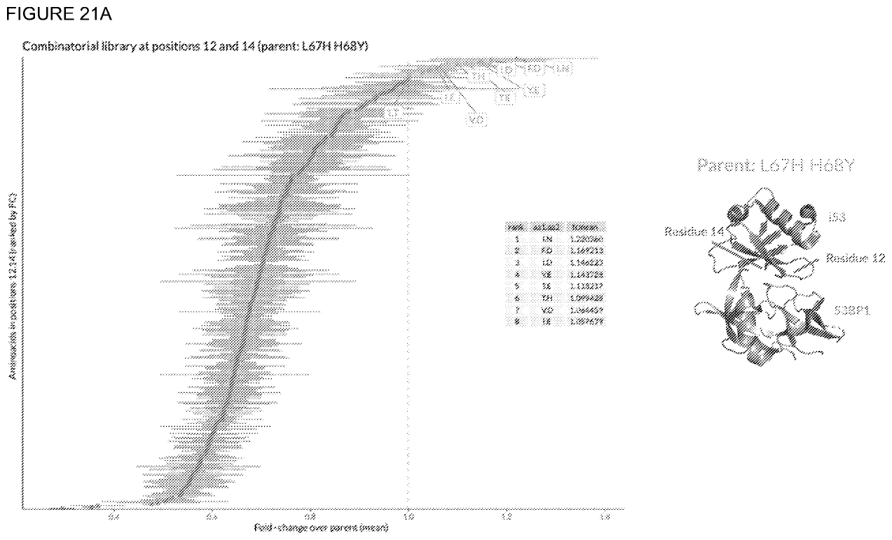

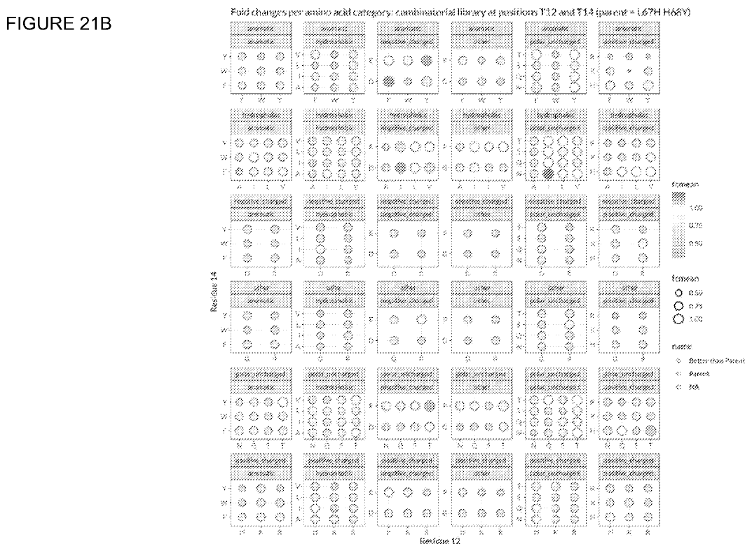

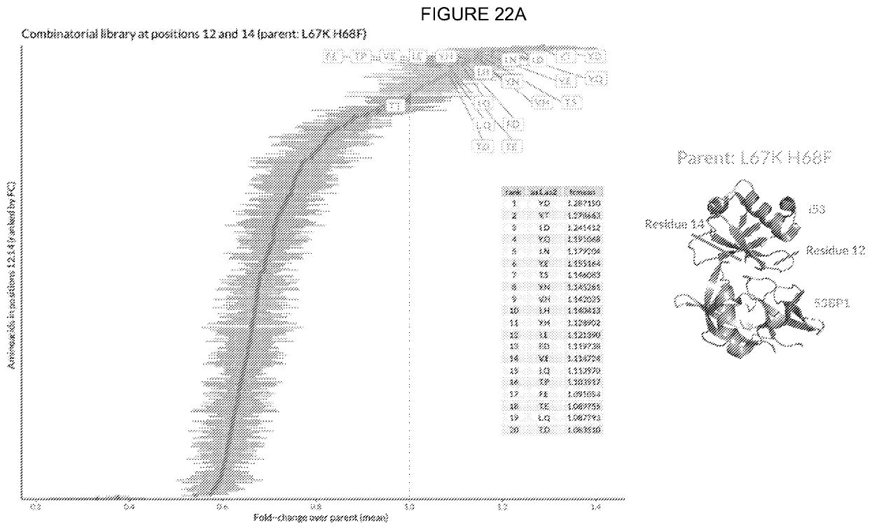

Top hits from the combinatorial library at residues 12 and 14 using L67H as parent were validated via pooled lentiviral expression. Top variants identified from the combinatorial screen above were cloned individually into the lentiviral vector shown above; the resulting plasmids were pooled together along with controls (parental control L67H and negative control i53 dead mutant, “DM”=P67L L70V) to generate a small validation library. CD34+ HSPC cells were transduced using lentivirus packaged with this library and edited in triplicate at the HBB locus using HBB-UBC-GFP AAV (MOI=2500). As shown in FIG. 20, NGS analysis of the gDNA purified from sorted mCherry+GFP+ and mCherry+GFP-populations confirmed positive enrichment relative to L67H for the majority of the mutant variants tested.

Additional HDR boosting mutants of i53 were identified by iterating on the L67H.H68Y i53 variant and screening combinatorial libraries targeting additional residues at the 53bp1/i53 interface. A combinatorial library was designed to explore all amino acid combinations at positions 12 and 14 using L67H.H68Y as the parent sequence. CD34+ HSPC cells were transduced using lentivirus packaged with this combinatorial library and edited in triplicate at the HBB locus using HBB-UBC-GFP AAV (MOI=2500). As shown in FIG. 21A, NGS analysis of the gDNA purified from sorted mCherry+GFP+ and mCherry+GFP-populations indicated differential variant enrichment in the mCherry+GFP+ population relative to parent L67H.H68Y (T12 and T14, shown in blue). As shown in FIG. 21B, when analyzing the results by amino acid categories, similar trends among hits were observed as in the analogous libraries with parents L67R and L67H (FIGS. 18 and 20, i.e. pairing aromatic and/or hydrophobic amino acids at residue 12 with negative charge at residue 14), but some additional unique beneficial amino acid combinations emerged as hit relative to parent L67H.H68Y (i.e. T12I.T14N.L67H.H68Y).

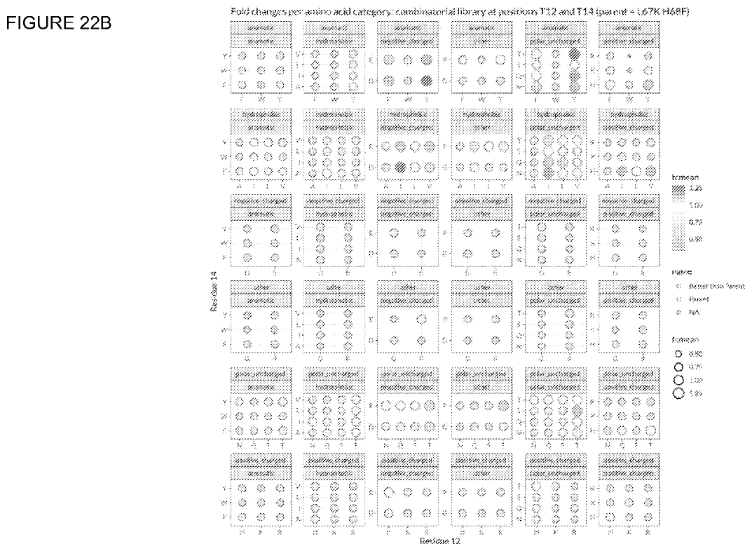

Additional HDR boosting mutants of i53 were identified by iterating on the L67K.H68F i53 variant and screening combinatorial libraries targeting additional residues at the 53bp1/i53 interface. A combinatorial library was designed to explore all amino acid combinations at positions 12 and 14 using L67K.H68F as the parent sequence. CD34+ HSPC cells were transduced using lentivirus packaged with this combinatorial library and edited in triplicate at the HBB locus using HBB-UBC-GFP AAV (MOI=2500). As shown in FIG. 22A, NGS analysis of the gDNA purified from sorted mCherry+GFP+ and mCherry+GFP-populations indicated differential variant enrichment in the mCherry+GFP+ population relative to parent L67K.H68F (T12 and T14, shown in blue). As shown in FIG. 22B, when analyzing the results by amino acid categories, similar trends among hits were again observed as in the analogous libraries with parents L67R, L67H, and L67H.H68Y (FIGS. 18, 20, and 22, i.e. pairing aromatic and/or hydrophobic amino acids at residue 12 with negative charge at residue 14), but additional beneficial amino acid combinations at residues 12 and 14 emerged as hits relative to parent L67K.H68F (i.e. pairing a tyrosine at position 12 with polar uncharged residues at position 14, including the parental threonine).

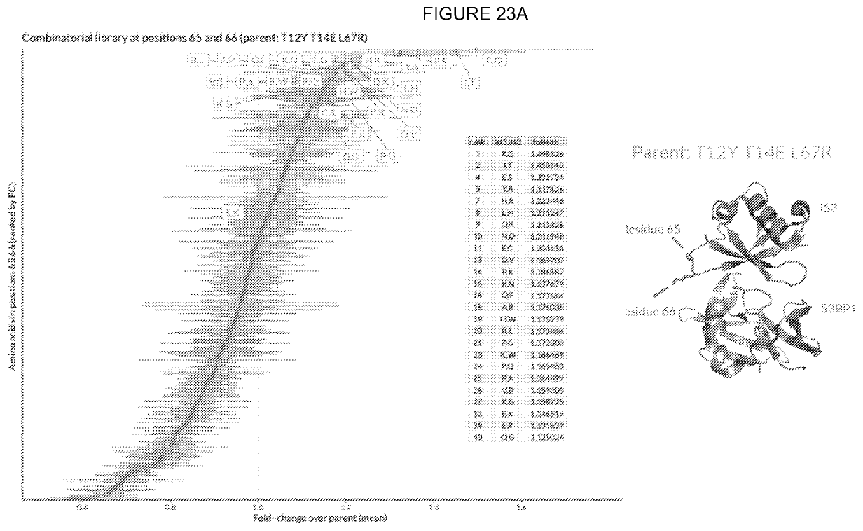

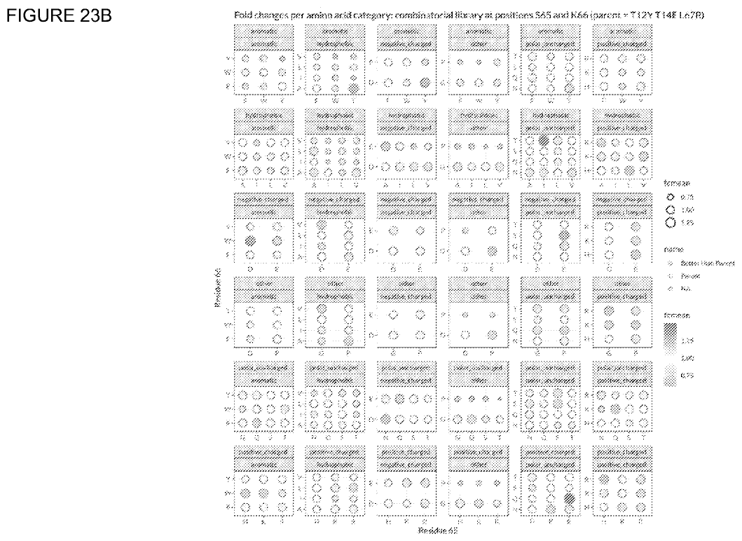

The top hits from the combinatorial library at residues 65 and 66 using T12Y.T14E.L67R as parent were validated via pooled lentiviral expression. Top variants identified from the combinatorial screen above were cloned individually into the lentiviral vector shown above; the resulting plasmids were pooled together along with controls (parental controls T12Y.T14E.L67R, L67R, and i53 and negative control i53 dead mutant, “DM”=P67L L70V) to generate a small validation library. CD34+ HSPC cells were transduced using lentivirus packaged with this library and edited in triplicate at the HBB locus using HBB-UBC-GFP AAV (MOI=2500). As shown in FIG. 23, NGS analysis of the gDNA purified from sorted mCherry+GFP+ and mCherry+GFP-populations confirmed positive enrichment for the majority of the variants tested relative to T12Y.T14E.L67R. A subset of these variants (including top hits identified in the screens shown above) were carried forward for additional validation and characterization.

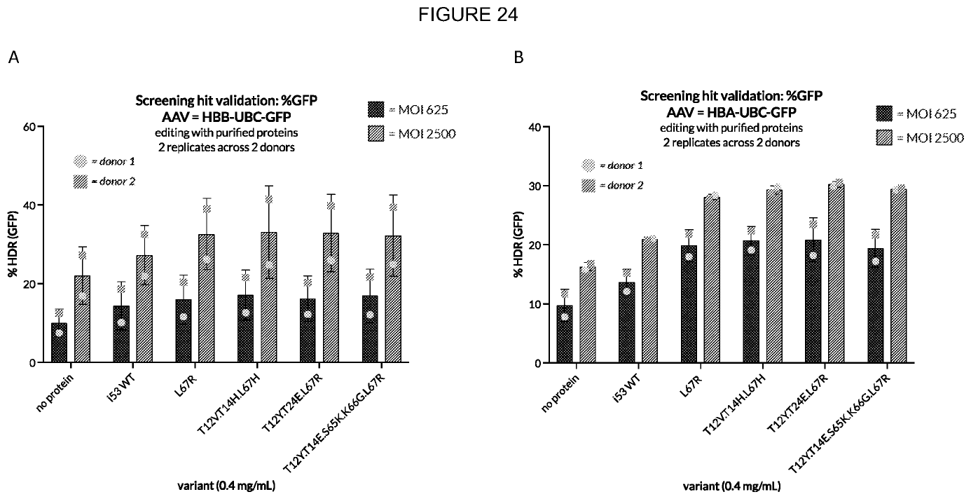

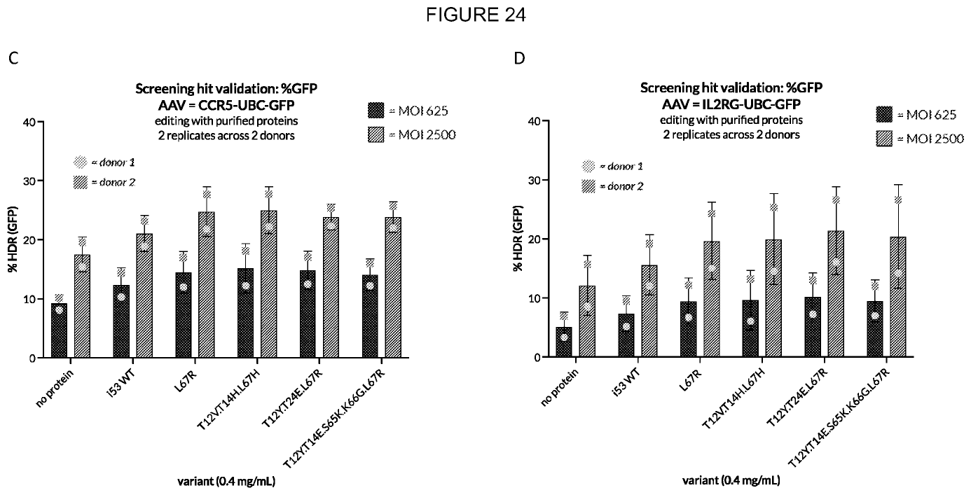

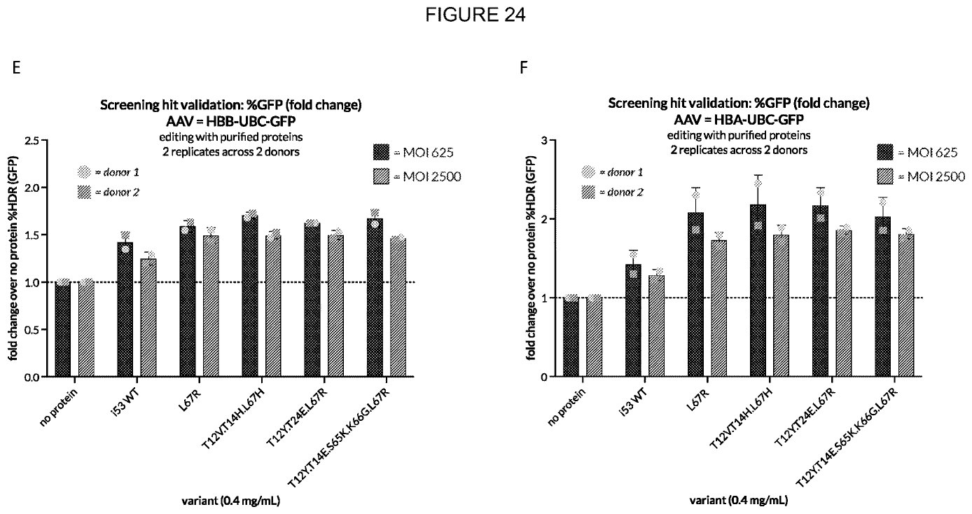

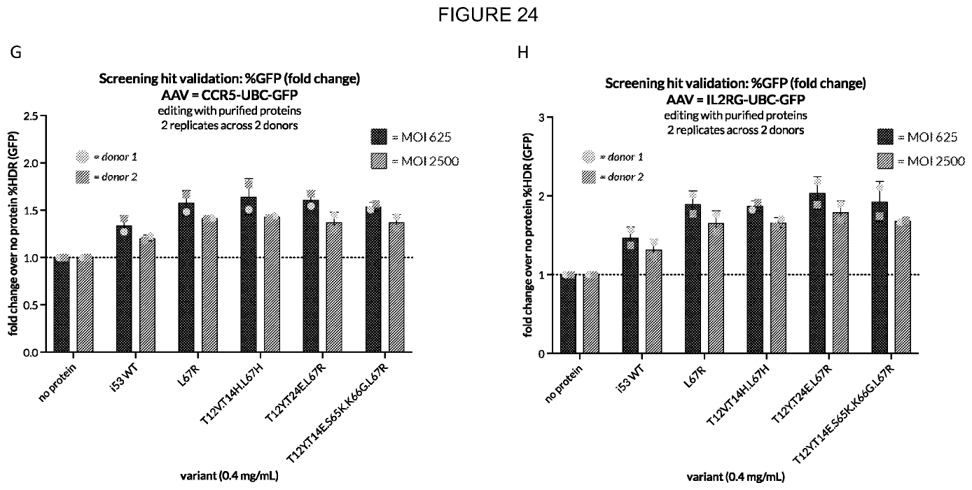

GFP knock-in to CD34+ HSPC cells was performed using AAV targeted at multiple clinically relevant loci, HBB,^{1,2} HBA,¹⁰ CCR^{5,11} and Il2RG¹² and three representative purified “hit” mutants of i53 identified in the above screens (T12Y.T14E.L67R, T12V.T14H.L67H, and T12Y.T14E.S65K.K66G.L67R). Protein variants (including L67R and a parental control i53) were incorporated into nucleofection solutions at concentrations of 0.4 mg/mL; post nucleofection, the cells were incubated with the AAV at MOIs of 625 and 2500. As shown in FIG. 24, the four i53 mutants tested (L67R, T12Y.T14E.L67R, T12V.T14H.L67H, and T12Y.T14E.S65K.K66G.L67R) were found to increase % GFP incorporation relative to the no protein control at the (A) HBB, (B) HBA, (C) CCR5, and (D) IL2RG loci in both CD34+ HSPC donors and at both MOIs tested. Incorporation of the parental i53 also increased % GFP knock-in relative to a no protein control, but at a consistently lower level than the i53 mutants identified in the functional screen. The fold increase in % HDR with the addition of the i53 variants differed across the loci tested, averaging (E) ~57% at HBB, (F) ~96% at HBA, (G) ~49% at CCR5, and (H) ~82% at IL2RG across the two donors and two MOIs (with a slightly higher fold change observed at the lower MOI relative to the high MOI for all AAVs tested). In comparison, i53 increased % HDR by only ~33% at HBB, ~36% at HBA, ~27% at CCR5, and ~39% at IL2RG. These results suggest that the additional potent variants identified by iterating on the L67/H68 variants using the screens described above can significantly boost HDR relative to i53 at multiple clinically relevant loci.

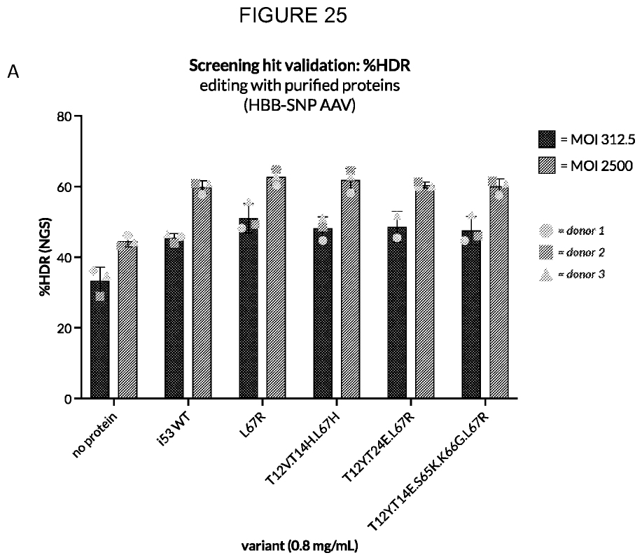

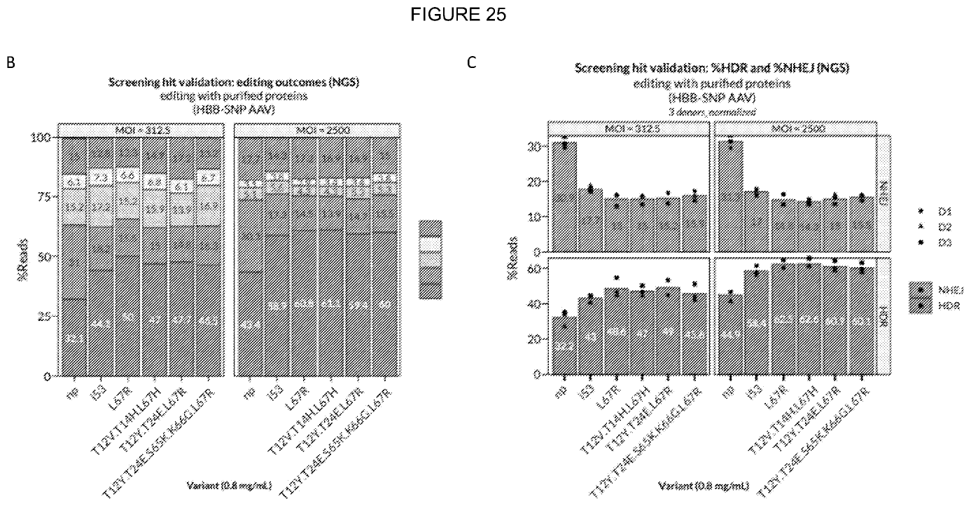

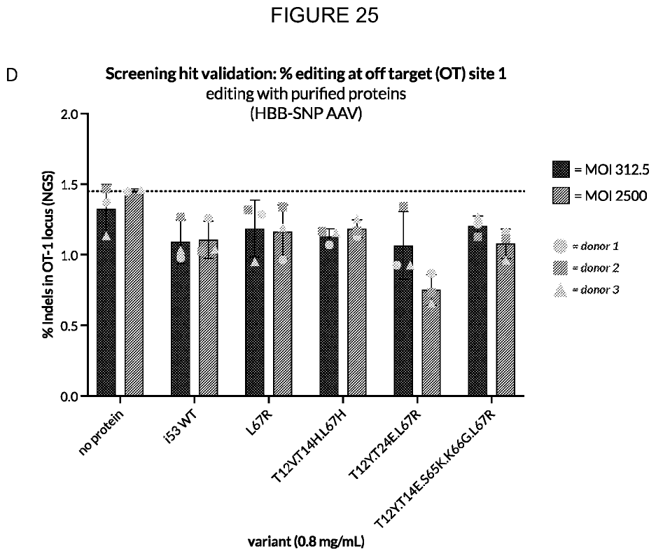

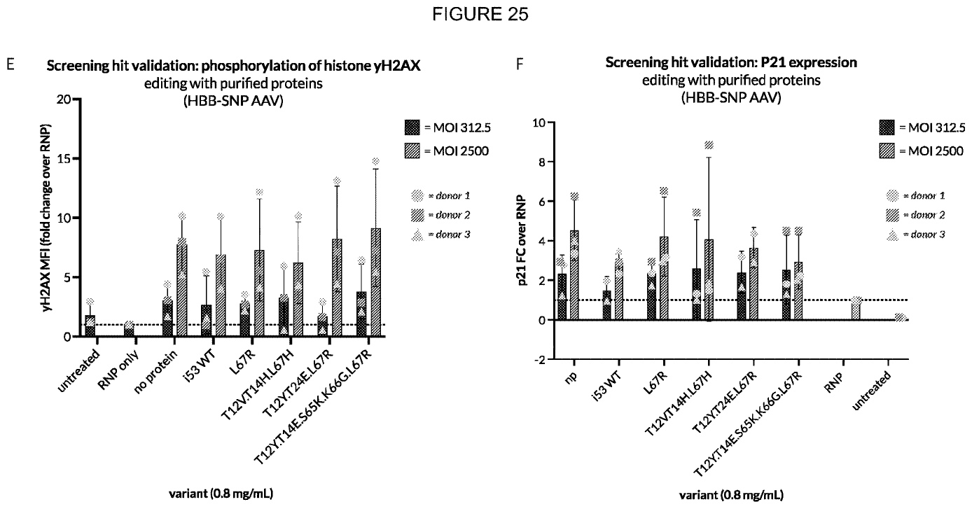

Editing of CD34+ HSPC cells from three donors was then performed using HBB-SNP AAV (sickle cell mutation correcting AAV template) 1,2 and three representative purified “hit” mutants of i53 identified in the above screens (T12Y.T14E.L67R, T12V.T14H.L67H, and T12Y.T14E.S65K.K66G.L67R). Protein variants (including L67R and a parental control i53) were incorporated into nucleofection solutions at concentrations of 0.8 mg/mL; post nucleofection, the cells were incubated with the AAV at MOIs of 312.5 and 2500. gDNA was extracted from the edited cells and analyzed via NGS. As shown in FIG. 25A, using these editing conditions, all variants tested were found to have a significant impact on rates of HDR corrected alleles at both 312.5 and 2500 MOI relative to the controls. As shown in FIG. 25B, edited alleles from each sample were further characterized by type of edit (unedited WT, MMEJ, NHEJ, HBD, or HDR) and the resulting analyses are shown. FIG. 25C provides the breakdown of NHEJ and HDR edit contributions, normalized by total edited alleles. The observed increases in % HDR with the i53 variants are accompanied by corresponding decreases in % NHEJ, with the i53 variants exhibiting more significant impact on % HDR and % NHEJ relative to i53. As shown in FIG. 25D, incorporation of the i53 variants and/or wild type i53 to the editing protocol was found to not significantly increase the frequency of INDELS at the off-target editing site OT-1, as assessed by NGS. Additionally, subsets of the edited cells were harvested to assess relative amounts of DDR by quantifying (FIG. 25E) yH2AX phosphorylation and (FIG. 25F) p21 expression. Higher amounts of yH2AX phosphorylation and p21 expression were observed in cells edited at 2500 MOI relative to those edited at 312.5 MOI. At both MOIs, however, the addition of the i53 variants and/or wild type i53 were not found to significantly increase the yH2AX and p21 response relative to the no protein control. These results again suggest that these high affinity i53 variants identified in above screens can be leveraged to maintain high levels of editing while enabling a beneficial decrease in AAV6 and DDR.

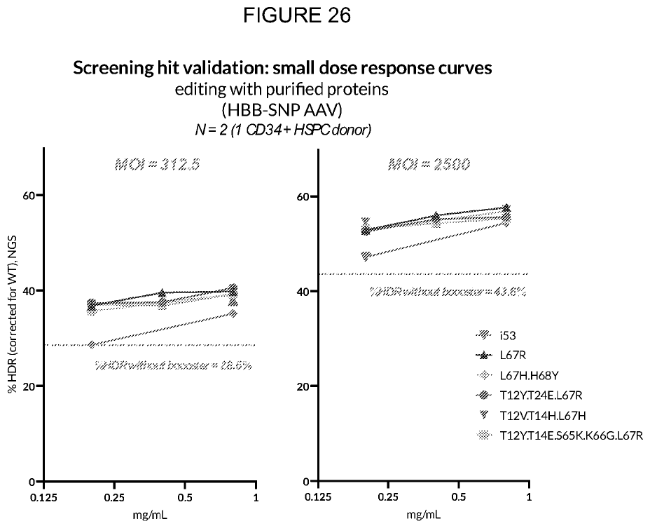

Dose response curves were generated for purified “hit” mutants of i53 identified in the above screens (L67R, L67H.H68Y, T12Y.T14E.L67R, T12V.T14H.L67H, and T12Y.T14E.S65K.K66G.L67R) relative to parental i53 using HBB-SNP AAV (sickle cell mutation correcting AAV template) 1,2. Protein variants were incorporated into nucleofection solutions at concentrations of 0.2, 0.4, and 0.8 mg/mL; post nucleofection, the cells were incubated with the AAV at an MOIs of 312.5 and 2500. In order to quantify editing outcomes, gDNA was extracted from the edited cells and analyzed via NGS. As shown in FIG. 26, Across all editing conditions, cells treated with the i53 variants maintained high levels of HDR corrected alleles (normalized by total edited alleles) relative to the control (cells edited without a protein-based additive, average % HDR across two replicates shown as grey dotted line) and i53 parent. Though exhibited a boost at 0.8 mg/mL, its effect on the levels of % HDR was found to decrease significantly as the protein concentrations decreased. These results confirm previous observations that the high affinity i53 variants identified in above screens are significantly more potent than parent i53.

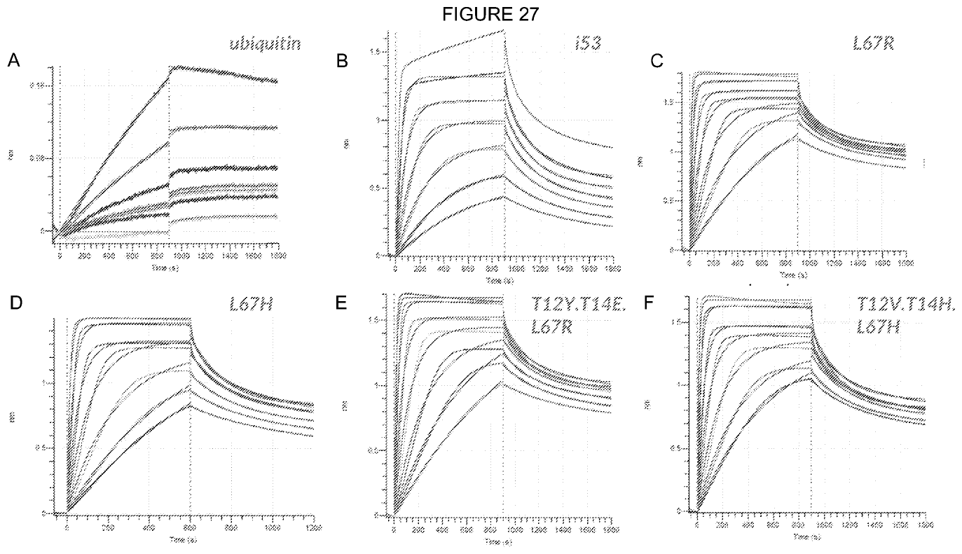

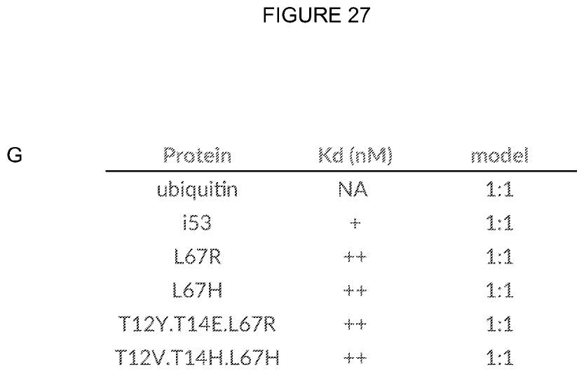

Biolayer Interferometry (BLI) was performed to measure binding kinetics of additional i53 mutants with immobilized 53BP1 Tudor domain. BLI was used to gather kinetic measurements (on- and off-rates) for i53 variant binding to biotin-labeled 53BP1 Tudor domain. The on- and off-rates measured at varying solution concentrations were used to calculate the dissociation constant (K_{D}) for each mutant listed. A 1:1 binding model was used for each K_{D} calculation. The + designation indicates a K_{D}>50 nM; ++ indicates a K_{D} between 5 and 50 nM. FIG. 27A provides ubiquitin binding to immobilized 53BP1 Tudor domain. FIG. 27B provides i53 binding to immobilized 53BP1 Tudor domain. FIG. 27C provides Mutant i53 L67R binding to immobilized 53BP1 Tudor domain. FIG. 27D provides Mutant i53 L67H binding to immobilized 53BP1 Tudor domain. FIG. 27E provides Mutant i53 T12Y.T14E.L67R binding to immobilized 53BP1 Tudor domain. FIG. 27F provides Mutant i53 T12V.T14H.L67H binding to immobilized 53BP1 Tudor domain. All purified “hit” mutants of i53 exhibit differential binding kinetics and a measurably increased rate of binding compared to parent i53, likely contributing to the increased impact these protein have on HDR levels relative to i53.

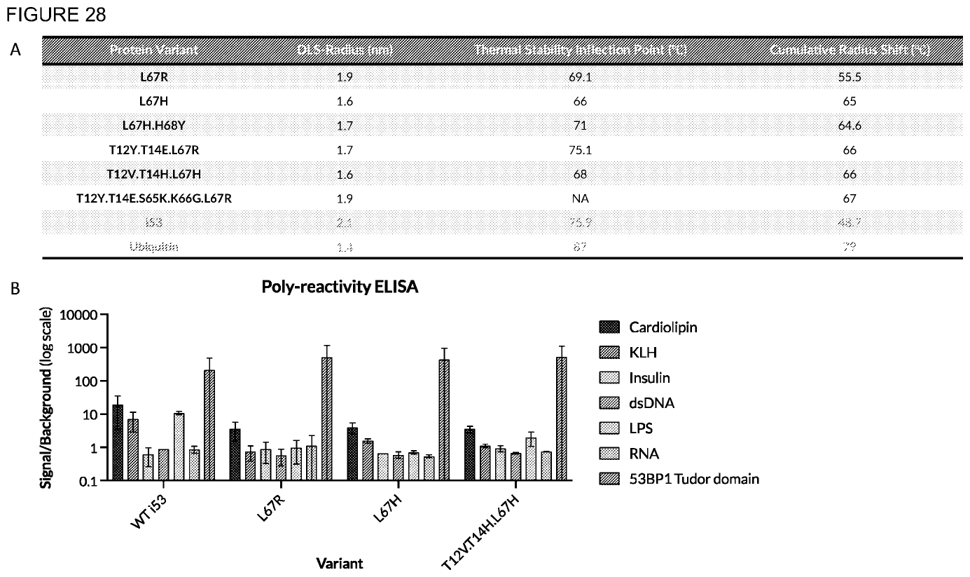

The stability and poly-reactivity of a subset of purified i53 variant proteins were further characterized. As shown in FIG. 28A, the i53 variant proteins were shown to be purified to high quality and show monodispersed properties when analyzed using dynamic light scattering (DLS). As shown in FIG. 28B, using an ELISA-based assay, the purified high affinity i53 variants were found to were found exhibit low poly-reactivity with highly specific binding to the 53BP1 Tudor domain.

### REFERENCES

1. Dever, D., Bak, R., Reinisch, A. et al. CRISPR/Cas9 β-globin gene targeting in human haematopoietic stem cells. Nature 539, 384-389 (2016).
2. Wilkinson, A. C., Dever, D. P., Baik, R. et al. Cas9-AAV6 gene correction of beta-globin in autologous HSCs improves sickle cell disease erythropoiesis in mice. Nat Commun 12, 686 (2021).
3. Clement, K., Rees, H., Canver, M. C. et al. CRISPResso2 provides accurate and rapid genome editing sequence analysis. Nat Biotechnol 37, 224-226 (2019).
4. Canny, M., Moatti, N., Wan, L. et al. Inhibition of 53BP1 favors homology-dependent DNA repair and increases CRISPR-Cas9 genome-editing efficiency. Nat Biotechnol 36, 95-102 (2018).
5. Emsley, P., Cowtan, K. Coot: model-building tools for molecular graphics. Acta Crystallogr D Biol Crystallogr 60, 2126-2132 (2004).
6. Ranjha, L., Howard, S. M. & Cejka, P. Main steps in DNA double-strand break repair: an introduction to homologous recombination and related processes. Chromosoma 127, 187-214 (2018).
7. Schiroli, G., Conti, A., Ferrari, S., et al. Precise gene editing preserves hematopoietic stem cell function following transient p53-Mediated DNA damage response. Cell Stem Cell 24, 551-565 (2019).
8. Nakamura, K., Sakai, W., Kawamoto, T., et al. Genetic dissection of vertebrate 53BP1: a major role in non-homologous end joining of DNA double strand breaks. DNA Repair 5, 741-749 (2006).
9. Bunting, S. F. Callén, E., Wong, N., et al. 53BP1 inhibits homologous recombination in Brca1-deficient cells by blocking resection of DNA breaks. Cell 141, 243-254 (2010).
10. Cromer, M. K., Camarena, J., Martin, R. M. et al. Gene replacement of α-globin with β-globin restores hemoglobin balance in β-thalassemia-derived hematopoietic stem and progenitor cells. Nat Med 27, 677-687 (2021).
11. Gomez-Ospina, N., Scharenberg, S. G., Mostrel, N. et al. Human genome-edited hematopoietic stem cells phenotypically correct Mucopolysaccharidosis type I. Nat Commun 10, 4045 (2019).
12. Pavel-Dinu, M., Wiebking, V., Dejene, B. T. et al. Gene correction for SCID-X1 in long-term hematopoietic stem cells. Nat Commun 10, 1634 (2019).
13. Cradick, T. J., Qiu, P., Lee, C. M., et al. COSMID: A Web-based Tool for Identifying and Validating CRISPR/Cas Off-target Sites. Mol Ther Nucleic Acids 3, e214 (2014).
14. Tsai, S., Nguyen, N., Malagon-Lopez, J. et al. CIRCLE-seq: a highly sensitive in vitro screen for genome-wide CRISPR-Cas9 nuclease off-targets. Nat Methods 14, 607-614 (2017).
15. Tsai, S., Zheng, Z., Nguyen, N. et al. GUIDE-seq enables genome-wide profiling of off-target cleavage by CRISPR-Cas nucleases. Nat Biotechnol 33, 187-197 (2015).

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

SEQUENCE LISTING

| SEQ | |
|---|---|
| ID | |
| NO | Sequence |
| 1 | MLIFVKTLTG KTITLEVEPS DTIENVKAKI QDKEGIPPDQ QRLAFAGKSL |
| | EDGRTLSDYN ILKDSKLHPL LRLR |
| 2 | MLIFVKTLTG KTITLEVEPS DTIENVKAKI QDKEGIPPDQ QRLAFAGKSL |
| | EDGRTLSDYN ILKDSKRHPL LRLR |
| 3 | MLIFVKTLTG KTITLEVEPS DTIENVKAKI QDKEGIPPDQ QRLAFAGKSL |
| | EDGRTLSDYN ILKDSKHHPL LRLR |
| 4 | MLIFVKTLTG KTITLEVEPS DTIENVKAKI QDKEGIPPDQ QRLAFAGKSL |
| | EDGRTLSDYN ILKDSKRWPL LRLR |
| 5 | MLIFVKTLTG KTITLEVEPS DTIENVKAKI QDKEGIPPDQ QRLAFAGKSL |
| | EDGRTLSDYN ILKDSHYPL LRLR |
| 6 | MLIFVKTLTG KTITLEVEPS DTIENVKAKI QDKEGIPPDQ QRLAFAGKSL |
| | EDGRTLSDYN ILKDSKHFPL LRLR |
| 7 | MLIFVKTLTG KTITLEVEPS DTIENVKAKI QDKEGIPPDQ QRLAFAGKSL |
| | EDGRTLSDYN ILKDSKLWPL LRLR |
| 8 | MLIFVKTLTG KTITLEVEPS DTIENVKAKI QDKEGIPPDQ QRLAFAGKSL |
| | EDGRTLSDYN ILKDSKLYPL LRLR |
| 9 | MLIFVKTLTG KTITLEVEPS DTIENVKAKI QDKEGIPPDQ QRLAFAGKSL |
| | EDGRTLSDYN ILKDSKLHPL LRLR |
| 10 | MLIFVKTLTG KTITLEVEPS DTIENVKAKI QDKEGIPPDQ QRLAFAGKSL |
| | EDGRTLSDYN ILKDSKHWPL LRLR |
| 11 | MLIFVKILTG KTITLEVEPS DTIENVKAKI QDKEGIPPDQ QRLAFAGKSL |
| | EDGRTLSDYN ILKDSKRYPL LRLR |
| 12 | MLIFVKILTG KTITLEVEPS DTIENVKAKI QDKEGIPPDQ QRLAFAGKSL |
| | EDGRTLSDYN ILKDSKRFPL LRLR |
| 13 | MLIFVKTLTG KTITLEVEPS DTIENVKAKI QDKEGIPPDQ QRLAFAGKSL |
| | EDGRTLSDYN ILKDSKSFPL LRLR |
| 14 | MLIFVKTLTG KTITLEVEPS DTIENVKAKI QDKEGIPPDQ QRLAFAGKSL |
| | EDGRTLSDYN ILKDSKTWPL LRLR |
| 15 | MLIFVKTLTG KTITLEVEPS DTIENVKAKI QDKEGIPPDQ QRLAFAGKSL |
| | EDGRTLSDYN ILKDSKSWPL LRLR |
| 16 | MLIFVKTLTG KTITLEVEPS DTIENVKAKI QDKEGIPPDQ QRLAFAGKSL |
| | EDGRTLSDYN ILKDSKQYPL LRLR |
| 17 | MLIFVKTLIG KTITLEVEPS DTIENVKAKI QDKEGIPPDQ QRLAFAGKSL |
| | EDGRTLSDYN ILKDSKNYPL LRLR |
| 18 | MLIFVKTLTG KTITLEVEPS DTIENVKAKI QDKEGIPPDQ QRLAFAGKSI |
| | EDGRTLSDYN ILKDSKQHPL LRLR |
| 19 | MLIFVKTLTG KTITLEVEPS DTIENVKAKI QDKEGIPPDQ QRLAFAGKSL |
| | EDGRTLSDYN ILKDSKNHPL LRLR |
| 20 | MLIFVKTLTG KTITLEVEPS DTIENVKAKI QDKEGIPPDQ QRLAFAGKSL |
| | EDGRTLSDYN ILKDSKNWPL LRLR |
| 21 | MLIFVKTLTG KTITLEVEPS DTIENVKAKI QDKEGIPPDQ QRLAFAGKSL |
| | EDGRTLSDYN ILKDSKKYPL LRLR |
| 22 | MLIFVKTLTG KVIHLEVEPS DTIENVKAKI QDKEGIPPDQ QRLAFAGKSL |
| | EDGRTLSDYN ILKDSKHHPL LRLR |
| 23 | MLIFVKTLTG KYIELEVEPS DTIENVKAKI QDKEGIPPDQ QRLAFAGKSL |
| | EDGRTLSDYN ILKDSKRHPL LRLR |
| 24 | MLIFVKTLTG KYIELEVEPS DTIENVKAKI QDKEGIPPDQ QRLAFAGKSL |
| | EDGRTLSDYN ILKDKGRHPL LRLR |
| 25 | MLIFVKTLTGKLIDLEVEPSDTIENVKAKIQDKEGIPPDQQRLAFAGKSLEDGRTLSD |
| | YNILKDSKRHPLLRLR |
| 26 | MLIFVKTLIGKIIELEVEPSDTIENVKAKIQDKEGIPPDQQRLAFAGKSLEDGRTLSD |
| | YNILKDSKRHPLLRLR |
| 27 | MLIFVKTLTGKIIDLEVEPSDTIENVKAKIQDKEGIPPDQQRLAFAGKSLEDGRTLSD |
| | YNILKDSKRHPLLRLR |
| 28 | MLIFVKTLTGKTIHLEVEPSDTIENVKAKIQDKEGIPPDQQRLAFAGKSLEDGRTLSD |
| | YNILKDSKRHPLLRLR |
| 29 | MLIFVKTLTGKTIPLEVEPSDTIENVKAKIQDKEGIPPDQQRLAFAGKSLEDGRTLSD |
| | YNILKDSKRHPLLRLR |
| 30 | MLIFVKTLTGKYIHLEVEPSDTIENVKAKIQDKEGIPPDQQRLAFAGKSLEDGRTLSD |
| | YNILKDSKRHPLLRLR |
| 31 | MLIFVKTLTGKIIPLEVEPSDTIENVKAKIQDKEGIPPDQQRLAFAGKSLEDGRTLSD |
| | YNILKDSKRHPLLRLR |
| 32 | MLIFVKTLTGKVIDLEVEPSDTIENVKAKIQDKEGIPPDQQRLAFAGKSLEDGRTLSD |
| | YNILKDSKRHPLLRLR |
| 33 | MLIFVKTLTGKTIDLEVEPSDTIENVKAKIQDKEGIPPDQQRLAFAGKSLEDGRTLSD |
| | YNILKDSKHHPLLRLR |
| 34 | MLIFVKTLTGKTIELEVEPSDTIENVKAKIQDKEGIPPDQQRLAFAGKSLEDGRTLSD |
| | YNILKDSKHHPLLRLR |
| 35 | MLIFVKTLTGKTIHLEVEPSDTIENVKAKIQDKEGIPPDQQRLAFAGKSLEDGRTLSD |
| | YNILKDSKHHPLLRLR |
| 36 | MLIFVKTLTGKYINLEVEPSDTIENVKAKIQDKEGIPPDQQRLAFAGKSLEDGRTLSD |
| | YNILKDSKHHPLLRLR |
| 37 | MLIFVKTLTGKYIELEVEPSDTIENVKAKIQDKEGIPPDQQRLAFAGKSLEDGRTLSD |
| | YNILKDSKHHPLLRLR |
| 38 | MLIFVKTLTGKLIHLEVEPSDTIENVKAKIQDKEGIPPDQQRLAFAGKSLEDGRTLSD |
| | YNILKDSKHHPLLRLR |
| 39 | MLIFVKTLTGKYIDLEVEPSDTIENVKAKIQDKEGIPPDQQRLAFAGKSLEDGRTLSD |
| | YNILKDSKHHPLLRLR |
| 40 | MLIFVKTLIGKYIHLEVEPSDTIENVKAKIQDKEGIPPDQQRLAFAGKSLEDGRTLSD |
| | YNILKDSKHHPLLRLR |
| 41 | MLIFVKTLTGKFIELEVEPSDTIENVKAKIQDKEGIPPDQQRLAFAGKSLEDGRTLSD |
| | YNILKDSKHHPLLRLR |
| 42 | MLIFVKTLTGKIINLEVEPSDTIENVKAKIQDKEGIPPDQQRLAFAGKSLEDGRTLSD |
| | YNILKDSKHYPLLRLR |
| 43 | MLIFVKTLTGKFIDLEVEPSDTIENVKAKIQDKEGIPPDQQRLAFAGKSLEDGRTLSD |
| | YNILKDSKHYPLLRLR |
| 44 | MLIFVKTLTGKIIDLEVEPSDTIENVKAKIQDKEGIPPDQQRLAFAGKSLEDGRTLSD |
| | YNILKDSKHYPLLRLR |
| 45 | MLIFVKTLTGKYIELEVEPSDTIENVKAKIQDKEGIPPDQQRLAFAGKSLEDGRTLSD |
| | YNILKDSKHYPLLRLR |
| 46 | MLIFVKTLTGKTIELEVEPSDTIENVKAKIQDKEGIPPDQQRLAFAGKSLEDGRTLSD |
| | YNILKDSKHYPLLRLR |
| 47 | MLIFVKTLTGKTIHLEVEPSDTIENVKAKIQDKEGIPPDQQRLAFAGKSLEDGRTLSD |
| | YNILKDSKHYPLLRLR |
| 48 | MLIFVKTLTGKYIDLEVEPSDTIENVKAKIQDKEGIPPDQQRLAFAGKSLEDGRTLSD |
| | YNILKDSKKFPLLRLR |
| 49 | MLIFVKTLTGKYITLEVEPSDTIENVKAKIQDKEGIPPDQQRLAFAGKSLEDGRTLSD |
| | YNILKDSKKFPLLRLR |
| 50 | MLIFVKTLTGKIIDLEVEPSDTIENVKAKIQDKEGIPPDQQRLAFAGKSLEDGRTLSD |
| | YNILKDSKKFPLLRLR |
| 51 | MLIFVKTLTGKYIQLEVEPSDTIENVKAKIQDKEGIPPDQQRLAFAGKSLEDGRTLSD |
| | YNILKDSKKFPLLRLR |
| 52 | MLIFVKTLTGKIINLEVEPSDTIENVKAKIQDKEGIPPDQQRLAFAGKSLEDGRTLSD |
| | YNILKDSKKFPLLRLR |
| 53 | MLIFVKTLTGKYIELEVEPSDTIENVKAKIQDKEGIPPDQQRLAFAGKSLEDGRTLSD |
| | YNILKDSKKFPLLRLR |
| 54 | MLIFVKTLTGKTISLEVEPSDTIENVKAKIQDKEGIPPDQQRLAFAGKSLEDGRTLSD |
| | YNILKDSKKFPLLRLR |
| 55 | MLIFVKTLTGKYINLEVEPSDTIENVKAKIQDKEGIPPDQQRLAFAGKSLEDGRTLSD |
| | YNILKDSKKFPLLRLR |
| 56 | MLIFVKTLTGKVIHLEVEPSDTIENVKAKIQDKEGIPPDQQRLAFAGKSLEDGRTLSD |
| | YNILKDSKKFPLLRLR |
| 57 | MLIFVKTLTGKIIHLEVEPSDTIENVKAKIQDKEGIPPDQQRLAFAGKSLEDGRTLSD |
| | YNILKDSKKFPLLRLR |
| 58 | MLIFVKTLTGKYIHLEVEPSDTIENVKAKIQDKEGIPPDQQRLAFAGKSLEDGRTLSD |
| | YNILKDSKKFPLLRLR |
| 59 | MLIFVKTLTGKIIELEVEPSDTIENVKAKIQDKEGIPPDQQRLAFAGKSLEDGRTLSD |
| | YNILKDSKKFPLLRLR |
| 60 | MLIFVKTLTGKYIELEVEPSDTIENVKAKIQDKEGIPPDQQRLAFAGKSLEDGRTLSD |
| | YNILKDRQRHPLLRLR |
| 61 | MLIFVKTLTGKYIELEVEPSDTIENVKAKIQDKEGIPPDQQRLAFAGKSLEDGRTLSD |
| | YNILKDITRHPLLRLR |
| 62 | MLIFVKTLTGKYIELEVEPSDTIENVKAKIQDKEGIPPDQQRLAFAGKSLEDGRTLSD |
| | YNILKDESRHPLLRLR |
| 63 | MLIFVKTLTGKTITLEVEPSDTIENVKAKIQDKEGIPPDQQRLAFAGKSLEDGRTLSD |
| | YNILKDEGRHPLLRLR |
| 64 | MLIFVKTLTGKYIELEVEPSDTIENVKAKIQDKEGIPPDQQRLAFAGKSLEDGRTLSD |
| | YNILKDNDRHPLLRLR |
| 65 | MLIFVKTLTGKYIELEVEPSDTIENVKAKIQDKEGIPPDQQRLAFAGKSLEDGRTLSD |
| | YNILKDPGRHPLLRLR |
| 66 | MLIFVKTLTGKYIELEVEPSDTIENVKAKIQDKEGIPPDQQRLAFAGKSLEDGRTLSD |
| | YNILKDHWRHPLLRLR |
| 67 | MLIFVKTLTGKYIELEVEPSDTIENVKAKIQDKEGIPPDQQRLAFAGKSLEDGRTLSD |
| | YNILKDVGRHPLLRLR |
| 68 | MLIFVKTLTGKYIELEVEPSDTIENVKAKIQDKEGIPPDQQRLAFAGKSLEDGRTLSD |
| | YNILKDYARHPLLRLR |
| 69 | MLIFVKTLTGKYIELEVEPSDTIENVKAKIQDKEGIPPDQQRLAFAGKSLEDGRTLSD |
| | YNILKDHRRHPLLRLR |
| 70 | MLIFVKTLTGKYIELEVEPSDTIENVKAKIQDKEGIPPDQQRLAFAGKSLEDGRTLSD |
| | YNILKDLHRHPLLRLR |
| 71 | MLIFVKTLTGKYIELEVEPSDTIENVKAKIQDKEGIPPDQQRLAFAGKSLEDGRTLSD |
| | YNILKDQKRHPLLRLR |
| 72 | MLIFVKTLTGKYIELEVEPSDTIENVKAKIQDKEGIPPDQQRLAFAGKSLEDGRTLSD |
| | YNILKDDVRHPLLRLR |
| 73 | gtcatcgctattaccatggtgatgcggttttggcagtacatcaatgggcgtggatagc |
| | ggtttgactcacggggatttccaagtctccaccccattgacgtcaatgggagtttgtt |
| | ttggcaccaaaatcaacgggactttccaaaatgtcgtaacaactccgccccattgacg |
| | caaatgggcggtaggcgtgtacggtgggaggtctatataagcagagctcgtttagtga |
| | accggggtctctctggttagaccagatctgagcctgggagctctctggctaactaggg |
| | aacccactgcttaagcctcaataaagcttgccttgagtgcttcaagtagtgtgtgccc |
| | gtctgttgtgtgactctggtaactagagatccctcagacccttttagtcagtgtggaa |
| | aatctctagcagtggcgcccgaacagggacctgaaagcgaaagggaaaccagagctct |
| | ctcgacgcaggactcggcttgctgaagcgcgcacggcaagaggcgaggggcggcgact |
| | ggtgagtacgccaaaaattttgactagcggaggctagaaggagagagatgggtgcgag |
| | agcgtcagtattaagcgggggagaattagatcgcgatgggaaaaaattcggttaaggc |
| | cagggggaaagaaaaaatataaattaaaacatatagtatgggcaagcagggagctaga |
| | acgattcgcagttaatcctggcctgttagaaacatcagaaggctgtagacaaatactg |
| | ggacagctacaaccatcccttcagacaggatcagaagaacttagatcattatataata |
| | cagtagcaaccctctattgtgtgcatcaaaggatagagataaaagacaccaaggaagc |
| | tttagacaagatagaggaagagcaaaacaaaagtaagaccaccgcacagcaagcggcc |
| | gctgatcttcagacctggaggaggagatatgagggacaattggagaagtgaattatat |
| | aaatataaagtagtaaaaattgaaccattaggagtagcacccaccaaggcaaagagaa |
| | gagtggtgcagagagaaaaaagagcagtgggaataggagctttgttccttgggttctt |
| | gggagcagcaggaagcactatgggcgcagcctcaatgacgctgacggtacaggccaga |
| | caattattgtctggtatagtgcagcagcagaacaatttgctgagggctattgaggcgc |
| | aacagcatctgttgcaactcacagtctggggcatcaagcagctccaggcaagaatcct |
| | ggctgtggaaagatacctaaaggatcaacagctcctggggatttggggttgctctgga |
| | aaactcatttgcaccactgctgtgccttggaatgctagttggagtaataaatctctgg |
| | aacagatttggaatcacacgacctggatggagtgggacagagaaattaacaattacac |
| | aagcttaatacactccttaattgaagaatcgcaaaaccagcaagaaaagaatgaacaa |
| | gaattattggaattagataaatgggcaagtttgtggaattggtttaacataacaaatt |
| | ggctgtggtatataaaattattcataatgatagtaggaggcttggtaggtttaagaat |
| | agtttttgctgtactttctatagtgaatagagttaggcagggatattcaccattatcg |
| | tttcagacccacctcccaaccccgaggggacccgacaggcccgaaggaatagaagaag |
| | aaggtggagagagagacagagacagatccattcgattagtgaacggatctcgacggta |
| | tcggttaacttttaaaagaaaaggggggattggggggtacagtgcaggggaaagaata |
| | gtagacataatagcaacagacatacaaactaaagaattacaaaaacaaattacaaaaa |
| | ttcaaaattttggctcccgatcgttgcgttacacacacaattactgctgatcgagtgt |
| | agccttcgaatgaaagaccccacctgtaggtttggcaagatagctgcagtaacgccat |
| | tttgcaaggcatggaaaaataccaaaccaagaatagagaagttcagatcaagggcggg |
| | tacatgaaaatagctaacgttgggccaaacaggatatctgcggtgagcagtttcggcc |
| | ccggcccggggccaagaacagatggtcaccgcagtttcggccccggcccgaggccaag |
| | aacagatggtccccagatatggcccaaccctcagcagtttcttaagacccatcagatg |
| | tttccaggctcccccaaggacctgaaatgaccctgcgccttatttgaattaaccaatc |
| | agcctgcttctcgcttctgttcgcgcgcttctgcttcccgagctctataaaagagctc |
| | acaacccctcactcggcgcgccagtcctccgattgactgagtcgccctgatcattgtc |
| | gatcctaccatccactcgacacacccgccaggatccccagcggccgcgccatctagag |
| | catgcatgggagtggggaaggtcgcggctccctgttgacttgtggcgatgtagaagag |
| | aacccaggtccagtgagcaagggcgaggaggataacatggccatcatcaaggagttca |
| | tgcgcttcaaggtgcacatggagggctccgtgaacggccacgagttcgagatcgaggg |
| | cgagggcgagggccgcccctacgagggcacccagaccgccaagctgaaggtgaccaag |
| | ggtggccccctgcccttcgcctgggacatcctgtcccctcagttcatgtacggctcca |
| | aggcctacgtgaagcaccccgccgacatccccgactacttgaagctgtccttccccga |
| | gggcttcaagtgggagcgcgtgatgaacttcgaggacggcggcgtggtgaccgtgacc |
| | caggactcctccctgcaggacggcgagttcatctacaaggtgaagctgcgcggcacca |
| | acttcccctccgacggccccgtaatgcagaagaagaccatgggctgggaggcctcctc |
| | cgagcggatgtaccccgaggacggcgccctgaagggcgagatcaagcagaggctgaag |
| | ctgaaggacggcggccactacgacgctgaggtcaagaccacctacaaggccaagaagc |
| | ccgtgcagctgcccggcgcctacaacgtcaacatcaagttggacatcacctcccacaa |
| | cgaggactacaccatcgtggaacagtacgaacgcgccgagggccgccactccaccggc |
| | ggcatggacgagctgtacaagtgagctagcgactatcatatgcttaccgtaatcaacc |
| | tctggattacaaaatttgtgaaagattgactggtattcttaactatgttgctcctttt |
| | acgctatgtggatacgctgctttaatgcctttgtatcatgctattgcttcccgtatgg |
| | ctttcattttctcctccttgtataaatcctggttgctgtctctttatgaggagttgtg |
| | gcccgttgtcaggcaacgtggcgtggtgtgcactgtgtttgctgacgcaacccccact |
| | ggttggggcattgccaccacctgtcagctcctttccgggactttcgctttccccctcc |
| | ctattgccacggcggaactcatcgccgcctgccttgcccgctgctggacaggggctcg |
| | gctgttgggcactgacaattccgtggtgttgtcggggaaatcatcgtcctttccttgg |
| | ctgctcgcctgtgttgccacctggattctgcgcgggacgtccttctgctacgtccctt |
| | cggccctcaatccagcggaccttccttcccgcggcctgctgccggctctgcggcctct |
| | tccgcgtcttcgccttcgccctcagacgagtcggatctccctttgggccgcctccccg |
| | cactagtgtctcgagggcccgcatcgaattcgagctcggtacctttaagaccaatgac |
| | ttacaaggcagctgtagatcttagccactttttaaaagaaaaggggggactggaaggg |
| | ctaattcactcccaacgaagacaagatctgctttttgcttgtactgggtctctctggt |
| | tagaccagatctgagcctgggagctctctggctaactagggaacccactgcttaagcc |
| | tcaataaagcttgccttgagtgcttcaagtagtgtgtgcccgtctgttgtgtgactct |
| | ggtaactagagatccctcagacccttttagtcagtgtggaaaatctctagcagtcctg |
| | gccaacgtgagcaccgtgctgacctccaaatatcgttaagctggagcctgggagccgg |
| | cctggccctccgccccccccacccccgcagcccacccctggtctttgaataaagtctg |
| | agtgagtggccgacagtgcccgtggagttctcgtgacctgaggtgcagggccggcgct |
| | agggacacgtccgtgcacgtgccgaggccccctgtgcagctgcaagggacaggcctag |
| | ccctgcaggcctaactccgcccatcccgcccctaactccgcccagttccgcccattct |
| | ccgcctcatggctgactaattttttttatttatgcagaggccgaggccgcctcggcct |
| | ctgagctattccagaagtagtgaggacgcttttttggaggccgaggcttttgcaaaga |
| | tcgaacaagagacaggacctgcaggttaattaaatttaaatcatgtgagcaaaaggcc |
| | agcaaaaggccaggaaccgtaaaaaggccgcgttgctggcgtttttccataggctccg |
| | cccccctgacgagcatcacaaaaatcgacgctcaagtcagaggtggcgaaacccgaca |
| | ggactataaagataccaggcgtttccccctggaagctccctcgtgcgctctcctgttc |
| | cgaccctgccgcttaccggatacctgtccgcctttctcccttcgggaagcgtggcgct |
| | ttctcatagctcacgctgtaggtatctcagttcggtgtaggtcgttcgctccaagctg |
| | ggctgtgtgcacgaaccccccgttcagcccgaccgctgcgccttatccggtaactatc |
| | gtcttgagtccaacccggtaagacacgacttatcgccactggcagcagccactggtaa |
| | caggattagcagagcgaggtatgtaggcggtgctacagagttcttgaagtggtggcct |
| | aactacggctacactagaagaacagtatttggtatctgcgctctgctgaagccagtta |
| | ccttcggaaaaagagttggtagctcttgatccggcaaacaaaccaccgctggtagcgg |
| | tggtttttttgtttgcaagcagcagattacgcgcagaaaaaaaggatctcaagaagat |
| | cctttgatcttttctacggggtctgacgctcagtggaacgaaaactcacgttaaggga |
| | ttttggtcatgagattatcaaaaaggatcttcacctagatccttttaaattaaaaatg |
| | aagttttaaatcaatctaaagtatatatgagtaaacttggtctgacagttaccaatgc |
| | ttaatcagtgaggcacctatctcagcgatctgtctatttcgttcatccatagttgcat |
| | ttaaatggccggcctggcgcgccgtttaaacctagatattgatagtctgatcggtcaa |
| | cgtataatcgagtcctagcttttgcaaacatctatcaagagacaggatcagcaggagg |
| | ctttcgcatgagtattcaacatttccgtgtcgcccttattcccttttttgcggcattt |
| | tgccttcctgtttttgctcacccagaaacgctggtgaaagtaaaagatgctgaagatc |
| | agttgggtgcgcgagtgggttacatcgaactggatctcaacagcggtaagatccttga |
| | gagttttcgccccgaagaacgctttccaatgatgagcacttttaaagttctgctatgt |
| | ggcgcggtattatcccgtattgacgccgggcaagagcaactcggtcgccgcatacact |
| | attctcagaatgacttggttgagtattcaccagtcacagaaaagcatcttacggatgg |
| | catgacagtaagagaattatgcagtgctgccataaccatgagtgataacactgcggcc |
| | aacttacttctgacaacgattggaggaccgaaggagctaaccgcttttttgcacaaca |
| | tgggggatcatgtaactcgccttgatcgttgggaaccggagctgaatgaagccatacc |
| | aaacgacgagcgtgacaccacgatgcctgtagcaatggcaacaaccttgcgtaaacta |
| | ttaactggcgaactacttactctagcttcccggcaacagttgatagactggatggagg |
| | cggataaagttgcaggaccacttctgcgctcggcccttccggctggctggtttattgc |
| | tgataaatctggagccggtgagcgtgggtctcgcggtatcattgcagcactggggcca |
| | gatggtaagccctcccgtatcgtagttatctacacgacggggagtcaggcaactatgg |
| | atgaacgaaatagacagatcgctgagataggtgcctcactgattaagcattggtaacc |
| | gattctaggtgcattggcgcagaaaaaaatgcctgatgcgacgctgcgcgtcttatac |
| | tcccacatatgccagattcagcaacggatacggcttccccaacttgcccacttccata |
| | cgtgtcctccttaccagaaatttatccttaagatcccgaatcgtttaaacgcgatcgc |
| | agtaatcaattacggggtcattagttcatagcccatatatggagttccgcgttacata |
| | acttacggtaaatggcccgcctggctgaccgcccaacgacccccgcccattgacgtca |
| | ataatgacgtatgttcccatagtaacgccaatagggactttccattgacgtcaatggg |
| | tggagtatttacggtaaactgcccacttggcagtacatcaagtgtatcatatgccaag |
| | tacgccccctattgacgtcaatgacggtaaatggcccgcctggcattatgcccagtac |
| | atgaccttatgggactttcctacttggcagtacatctacgtatta |
| 74 | CTTGCCCCACAGGGCAGTAA |
| 75 | gctccagccggttccagctattgctttgtttacctgtttaaccagtatttacctagca |
| | agtcttccatcagatagcatttggagagctgggggtgtcacagtgaaccacgacctct |
| | aggccagtgggagagtcagtcacacaaactgtgagtccatgacttggggcttagccag |
| | cacccaccaccccacgcgccaccccacaaccccgggtagaggagtctgaatctggagc |
| | cgcccccagcccagccccgtgctttttgcgtcctggtgtttattccttcccggtgcct |
| | gtcactcaagcacactagtgactatcgccagagggaaagggagctgcaggaagcgagg |
| | ctggagagcaggaggggctctgcgcagaaattcttttgagttcctatgggccagggcg |
| | tccgggtgcgcgcattcctctccgccccaggattgggcgaagcctcccggctcgcact |
| | cgctcgcccgtgtgttccccgatcccgctggagtcgatgcgcgtccagcgcgtgccag |
| | gccggggcgggggtgcgggctgactttctccctcgctagggacgctccggcgcccgaa |
| | aggaaagggtggcgctgcgctccggggtgcacgagccgacagcgcccgaccccaacgg |
| | gccggccccgccagcgccgctaccgccctgcccccgggcgagcgggatgggcgggagt |
| | ggagtggcgggtggagggtggagacgtcctggcccccgccccgcgtgcacccccaggg |
| | gaggccgagcccgccgcccggccccgcgcaggccccgcccgggactcccctgcggtcc |
| | aggccgcgccccgggctccgcgccagccaatgagcgccgcccggccgggcgtgccccc |
| | gcgccccaagcataaaccctggcgcgctcgcggcccggcactcttctggtccccacag |
| | actcagagagaacccaccggcctccgcgccgggttttggcgcctcccgcgggcgcccc |
| | cctcctcacggcgagcgctgccacgtcagacgaagggcgcagcgagcgtcctgatcct |
| | tccgcccggacgctcaggacagcggcccgctgctcataagactcggccttagaacccc |
| | agtatcagcagaaggacattttaggacgggacttgggtgactctagggcactggtttt |
| | ctttccagagagcggaacaggcgaggaaaagtagtcccttctcggcgattctgcggag |
| | ggatctccgtggggcggtgaacgccgatgattatataaggacgcgccgggtgtggcac |
| | agctagttccgtcgcagccgggatttgggtcgcggttcttgtttgtggatcgctgtga |
| | tcgtcacttggtgagtagcgggctgctgggctggccggggctttcgtggccgccgggc |
| | cgctcggtgggacggaagcgtgtggagagaccgccaagggctgtagtctgggtccgcg |
| | agcaaggttgccctgaactgggggttggggggagcgcagcaaaatggcggctgttccc |
| | gagtcttgaatggaagacgcttgtgaggcgggctgtgaggtcgttgaaacaaggtggg |
| | gggcatggtgggcggcaagaacccaaggtcttgaggccttcgctaatgcgggaaagct |
| | cttattcgggtgagatgggctggggcaccatctggggaccctgacgtgaagtttgtca |
| | ctgactggagaactcggtttgtcgtctgttgcgggggcggcagttatggcggtgccgt |
| | tgggcagtgcacccgtacctttgggagcgcgcgccctcgtcgtgtcgtgacgtcaccc |
| | gttctgttggcttataatgcagggtggggccacctgccggtaggtgtgcggtaggctt |
| | ttctccgtcgcaggacgcagggttcgggcctagggtaggctctcctgaatcgacaggc |
| | gccggacctctggtgaggggagggataagtgaggcgtcagtttctttggtcggtttta |
| | tgtacctatcttcttaagtagctgaagctccggttttgaactatgcgctcggggttgg |
| | cgagtgtgttttgtgaagttttttaggcaccttttgaaatgtaatcatttgggtcaat |
| | atgtaattttcagtgttagactagtaaattgtccgctaaattctggccgtttttggct |
| | tttttgttagacggtaccgagctcttcgaaggatccatcgccaccatgcccgccatga |
| | agatcgagtgccgcatcaccggcaccctgaacggcgtggagttcgagctggtgggcgg |
| | cggagagggcacccccgagcagggccgcatgaccaacaagatgaagagcaccaaaggc |
| | gccctgaccttcagcccctacctgctgagccacgtgatgggctacggcttctaccact |
| | tcggcacctaccccagcggctacgagaaccccttcctgcacgccatcaacaacggcgg |
| | ctacaccaacacccgcatcgagaagtacgaggacggcggcgtgctgcacgtgagcttc |
| | agctaccgctacgaggccggccgcgtgatcggcgacttcaaggtggtgggcaccggct |
| | tccccgaggacagcgtgatcttcaccgacaagatcatccgcagcaacgccaccgtgga |
| | gcacctgcaccccatgggcgataacgtgctggtgggcagcttcgcccgcaccttcagc |
| | ctgcgcgacggcggctactacagcttcgtggtggacagccacatgcacttcaagagcg |
| | ccatccaccccagcatcctgcagaacgggggccccatgttcgccttccgccgcgtgga |
| | ggagctgcacagcaacaccgagctgggcatcgtggagtaccagcacgccttcaagacc |
| | cccatcgccttcgccagatctcgagtctagctcgagggcgcgcccgctgatcagcctc |
| | gacctgtgccttctagttgccagccatctgttgtttgcccctcccccgtgccttcctt |
| | gaccctggaaggtgccactcccactgtcctttcctaataaaatgaggaaattgcatcg |
| | cattgtctgagtaggtgtcattctattctggggggtggggtggggcaggacagcaagg |
| | gggaggattgggaagacaatagcaggcatgctggggatggccatgcttcttgcccctt |
| | gggcctccccccagcccctcctccccttcctgcacccgtacccccgtggtctttgaat |
| | aaagtctgagtgggcggcagcctgtgtgtgcctgagttttttccctcagcaaacgtgc |
| | caggcatgggcgtggacagcagctgggacacacatggctagaacctctctgcagctgg |
| | atagggtaggaaaaggcaggggcgggaggaggggatggaggagggaaagtggagccac |
| | cgcgaagtccagctggaaaaacgctggaccctagagtgctttgaggatgcatttgctc |
| | tttcccgagttttattcccagacttttcagattcaatgcaggtttgctgaaataatga |
| | atttatccatctttacgtttctgggcactctgtgccaagaactggctggctttctgcc |
| | tgggacgtcactggtttcccagaggtcctcccacatatgggtggtgggtaggtcagag |
| | aagtcccactccagcatggctgcattgatcccccatcgttcccactagtctccgtaaa |
| | acctcccagatacaggcacagtctagatgaaatcaggggtgcggggtgcaactgcagg |
| | ccccaggcaattcaataggggctctactttcacccccaggtcaccccagaatgctcac |
| | acaccagacactgacgccctggggctgtcaagatcaggcgtttgtctctgggcccagc |
| | tcagggcccagctcagcacccactcagctcccctgaggctggggagcctgtcccattg |
| | cgactggagaggagagcggggccacagaggcctggctagaaggtcccttctccctggt |
| | gtgtgttttctctctgctgagcaggcttgcagtgcctggggtatca |
| 76 | gctttcatgaattcccccaacagagccaagctctccatctagtggacagggaagctag |
| | cagcaaaccttcccttcactacaaaacttcattgcttggccaaaaagagagttaattc |
| | aatgtagacatctatgtaggcaattaaaaacctattgatgtataaaacagtttgcatt |
| | catggagggcaactaaatacattctaggactttataaaagatcactttttatttatgc |
| | acagggtggaacaagatggattatcaagtgtcaagtccaatctatgacatcaattatt |
| | atacatcggagccctgccaaaaaatcaatgtgaagcaaatcgcagcccgcctcctgcc |
| | tccgctctactcactggtgttcatctttggttttgtgggcaacatgctggtcatcctc |
| | atcctgataaactgcaaaaggctgaagagcatgactgacatctacctgctcaacctgg |
| | ccatctctgacctgtttttccttcttactgtccccttcggcctccgcgccgggttttg |
| | gcgcctcccgcgggcgcccccctcctcacggcgagcgctgccacgtcagacgaagggc |
| | gcagcgagcgtcctgatccttccgcccggacgctcaggacagcggcccgctgctcata |
| | agactcggccttagaaccccagtatcagcagaaggacattttaggacgggacttgggt |
| | gactctagggcactggttttctttccagagagcggaacaggcgaggaaaagtagtccc |
| | ttctcggcgattctgcggagggatctccgtggggcggtgaacgccgatgattatataa |
| | ggacgcgccgggtgtggcacagctagttccgtcgcagccgggatttgggtcgcggttc |
| | ttgtttgtggatcgctgtgatcgtcacttggtgagtagcgggctgctgggctggccgg |
| | ggctttcgtggccgccgggccgctcggtgggacggaagcgtgtggagagaccgccaag |
| | ggctgtagtctgggtccgcgagcaaggttgccctgaactgggggttggggggagcgca |
| | gcaaaatggcggctgttcccgagtcttgaatggaagacgcttgtgaggcgggctgtga |
| | ggtcgttgaaacaaggtggggggcatggtgggcggcaagaacccaaggtcttgaggcc |
| | ttcgctaatgcgggaaagctcttattcgggtgagatgggctggggcaccatctgggga |
| | ccctgacgtgaagtttgtcactgactggagaactcggtttgtcgtctgttgcgggggc |
| | ggcagttatggcggtgccgttgggcagtgcacccgtacctttgggagcgcgcgccctc |
| | gtcgtgtcgtgacgtcacccgttctgttggcttataatgcagggtggggccacctgcc |
| | ggtaggtgtgcggtaggcttttctccgtcgcaggacgcagggttcgggcctagggtag |
| | gctctcctgaatcgacaggcgccggacctctggtgaggggagggataagtgaggcgtc |
| | agtttctttggtcggttttatgtacctatcttcttaagtagctgaagctccggttttg |
| | aactatgcgctcggggttggcgagtgtgttttgtgaagttttttaggcaccttttgaa |
| | atgtaatcatttgggtcaatatgtaattttcagtgttagactagtaaattgtccgcta |
| | aattctggccgtttttggcttttttgttagacggtaccgagctcttcgaaggatccat |
| | cgccaccatgcccgccatgaagatcgagtgccgcatcaccggcaccctgaacggcgtg |
| | gagttcgagctggtgggcggcggagagggcacccccgagcagggccgcatgaccaaca |
| | agatgaagagcaccaaaggcgccctgaccttcagcccctacctgctgagccacgtgat |
| | gggctacggcttctaccacttcggcacctaccccagcggctacgagaaccccttcctg |
| | cacgccatcaacaacggcggctacaccaacacccgcatcgagaagtacgaggacggcg |
| | gcgtgctgcacgtgagcttcagctaccgctacgaggccggccgcgtgatcggcgactt |
| | caaggtggtgggcaccggcttccccgaggacagcgtgatcttcaccgacaagatcatc |
| | cgcagcaacgccaccgtggagcacctgcaccccatgggcgataacgtgctggtgggca |
| | gcttcgcccgcaccttcagcctgcgcgacggcggctactacagcttcgtggtggacag |
| | ccacatgcacttcaagagcgccatccaccccagcatcctgcagaacgggggccccatg |
| | ttcgccttccgccgcgtggaggagctgcacagcaacaccgagctgggcatcgtggagt |
| | accagcacgccttcaagacccccatcgccttcgccagatctcgagtctagctcgaggg |
| | cgcgcccgctgatcagcctcgacctgtgccttctagttgccagccatctgttgtttgc |
| | ccctcccccgtgccttccttgaccctggaaggtgccactcccactgtcctttcctaat |
| | aaaatgaggaaattgcatcgcattgtctgagtaggtgtcattctattctggggggtgg |
| | ggtggggcaggacagcaagggggaggattgggaagacaatagcaggcatgctggggat |
| | gcggtgggctctatggtgggctcactatgctgccgcccagtgggactttggaaataca |
| | atgtgtcaactcttgacagggctctattttataggcttcttctctggaatcttcttca |
| | tcatcctcctgacaatcgataggtacctggctgtcgtccatgctgtgtttgctttaaa |
| | agccaggacggtcacctttggggtggtgacaagtgtgatcacttgggtggtggctgtg |
| | tttgcgtctctcccaggaatcatctttaccagatctcaaaaagaaggtcttcattaca |
| | cctgcagctctcattttccatacagtcagtatcaattctggaagaatttccagacatt |
| | aaagatagtcatcttggggctggtcctgccgctgcttgtcatggtcatctgctactcg |
| | ggaatcctaaaaactctgcttcggtgtcgaaatgagaagaagaggcacagggctgtga |
| | ggcttatcttcaccatcatgattgtttattttctcttctgggctccctacaa |
| 77 | gcatggcatagaacggtgatgtcgggggtgggggttcagaacttccattatagaaggt |
| | aatgatttagaggagaaggtggttgagaatggtgctagtggtagtgaacagatccttc |
| | ccaggatctaggtgggctgaggatttttgagtctgtgacactattgtatatccagctt |
| | tagtttctgtttaccaccttacagcagcacctaatctcctagaggacttagcccgtgt |
| | cacacagcacatatttgccacaccctctgtaaagccctggtttataaggttctttcca |
| | ccggaagctatgacagaggaaacgtgtgggtggggaggggtagtgggtgagggaccca |
| | ggttcctgacacagacagactacacccagggaatgaagagcaagcgccatgtggcctc |
| | cgcgccgggttttggcgcctcccgcgggcgcccccctcctcacggcgagcgctgccac |
| | gtcagacgaagggcgcagcgagcgtcctgatccttccgcccggacgctcaggacagcg |
| | gcccgctgctcataagactcggccttagaaccccagtatcagcagaaggacattttag |
| | gacgggacttgggtgactctagggcactggttttctttccagagagcggaacaggcga |
| | ggaaaagtagtcccttctcggcgattctgcggagggatctccgtggggcggtgaacgc |
| | cgatgattatataaggacgcgccgggtgtggcacagctagttccgtcgcagccgggat |
| | ttgggtcgcggttcttgtttgtggatcgctgtgatcgtcacttggtgagtagcgggct |
| | gctgggctggccggggctttcgtggccgccgggccgctcggtgggacggaagcgtgtg |
| | gagagaccgccaagggctgtagtctgggtccgcgagcaaggttgccctgaactggggg |
| | ttggggggagcgcagcaaaatggcggctgttcccgagtcttgaatggaagacgcttgt |
| | gaggcgggctgtgaggtcgttgaaacaaggtggggggcatggtgggcggcaagaaccc |
| | aaggtcttgaggccttcgctaatgcgggaaagctcttattcgggtgagatgggctggg |
| | gcaccatctggggaccctgacgtgaagtttgtcactgactggagaactcggtttgtcg |
| | tctgttgcgggggcggcagttatggcggtgccgttgggcagtgcacccgtacctttgg |
| | gagcgcgcgccctcgtcgtgtcgtgacgtcacccgttctgttggcttataatgcaggg |
| | tggggccacctgccggtaggtgtgcggtaggcttttctccgtcgcaggacgcagggtt |
| | cgggcctagggtaggctctcctgaatcgacaggcgccggacctctggtgaggggaggg |
| | ataagtgaggcgtcagtttctttggtcggttttatgtacctatcttcttaagtagctg |
| | aagctccggttttgaactatgcgctcggggttggcgagtgtgttttgtgaagtttttt |
| | aggcaccttttgaaatgtaatcatttgggtcaatatgtaattttcagtgttagactag |
| | taaattgtccgctaaattctggccgtttttggcttttttgttagacggtaccgagctc |
| | ttcgaaggatccatcgccaccatgcccgccatgaagatcgagtgccgcatcaccggca |
| | ccctgaacggcgtggagttcgagctggtgggcggcggagagggcacccccgagcaggg |
| | ccgcatgaccaacaagatgaagagcaccaaaggcgccctgaccttcagcccctacctg |
| | ctgagccacgtgatgggctacggcttctaccacttcggcacctaccccagcggctacg |
| | agaaccccttcctgcacgccatcaacaacggcggctacaccaacacccgcatcgagaa |
| | gtacgaggacggcggcgtgctgcacgtgagcttcagctaccgctacgaggccggccgc |
| | gtgatcggcgacttcaaggtggtgggcaccggcttccccgaggacagcgtgatcttca |
| | ccgacaagatcatccgcagcaacgccaccgtggagcacctgcaccccatgggcgataa |
| | cgtgctggtgggcagcttcgcccgcaccttcagcctgcgcgacggcggctactacagc |
| | ttcgtggtggacagccacatgcacttcaagagcgccatccaccccagcatcctgcaga |
| | acgggggccccatgttcgccttccgccgcgtggaggagctgcacagcaacaccgagct |
| | gggcatcgtggagtaccagcacgccttcaagacccccatcgccttcgccagatctcga |
| | gtctagctcgagggcgcgcccgctgatcagcctcgacctgtgccttctagttgccagc |
| | catctgttgtttgcccctcccccgtgccttccttgaccctggaaggtgccactcccac |
| | tgtcctttcctaataaaatgaggaaattgcatcgcattgtctgagtaggtgtcattct |
| | attctggggggtggggtggggcaggacagcaagggggaggattgggaagacaatagca |
| | ggcatgctggggatgcggtgggctcctgcaggtgaagccatcattaccattcacatcc |
| | ctcttattcctgcagctgcccctgctgggagtggggctgaacacgacaattctgacgc |
| | ccaatgggaatgaagacaccacagctggtgggaaatctgggactggagggggctggtg |
| | agaagggtggctgtgggaaggggccgtacagagatctggtgcctgccactggccatta |
| | caatcatgtgggcagaattgaaaagtggagtgggaagggcaagggggagggttccctg |
| | cctcacgctacttcttctttctttcttgtttgtttgtttctttctttcttttgaggca |
| | gggtctcactatgttgcctaggctggtctcaaactcctggcctctagtgatcctcctg |
| | cctcagcctttcaaagcaccaggattacagacatgagcca |
| 78 | GGCAAGAAGCATGGCCACCG |
| 79 | GCAGCATAGTGAGCCCAGAA |
| 80 | TGGTAATGATGGCTTCAACA |

What is claimed:

1. A polypeptide comprising the amino acid sequence of SEQ ID NO: 1 having one or more modifications relative to SEQ ID NO: 1, wherein the one or more modifications comprise a modification at position 67 of SEQ ID NO: 1, a modification at position 68 of SEQ ID NO: 1, or a combination thereof.

2. A polypeptide comprising an amino acid sequence having at least 60% sequence identity to the amino acid sequence of SEQ ID NO 1, having one or more modifications relative to SEQ ID NO: 1, wherein the one or more modifications comprise a modification at position 67 of SEQ ID NO: 1, a modification at position 68 of SEQ ID NO: 1, or a combination thereof.

3. The polypeptide of any one of claims 1 to 2, wherein the modification at position 67 of SEQ ID NO: 1 is:
(a) Arg (L67R);
(b) His (L67H);
(c) Lys (L67K), provided that the polypeptide further comprises a modification at position 68 of SEQ ID NO: 1;
(d) Ser (L67S);
(e) Thr (L67T);
(f) Gln (L67Q);
(g) Asn (L67N); or
(h) His (L67H).

4. The polypeptide of any one of claims 1 to 3, wherein the modification at position 68 of SEQ ID NO: 1 is selected from the group consisting of:
(a) Trp (H68W);
(b) Tyr (H68Y); or
(c) Phe (H68F).

5. The polypeptide of any one of claims 1 to 2, wherein the one or more modification are selected from the group consisting of:
(a) an Arg at position 67 of SEQ ID NO: 1;
(b) a His at position 67 of SEQ ID NO: 1;
(c) a Lys at position 67 of SEQ ID NO: 1 (L67K), provided that the polypeptide further comprises a modification at position 68 of SEQ ID NO: 1;
(d) a Ser at position 67 of SEQ ID NO: 1 (L67S);
(e) a Thr at position 67 of SEQ ID NO: 1 (L67T);
(f) a Gln at position 67 of SEQ ID NO: 1 (L67Q);
(g) An Asn at position 67 of SEQ ID NO: 1 (L67N);
(h) a His at position 67 of SEQ ID NO: 1 (L67H);
(i) a Trp at position 68 of SEQ ID NO: 1 (H68W);
(j) a Tyr at position 68 of SEQ ID NO: 1 (H68Y);
(k) a Phe at position 68 of SEQ ID NO: 1 (H68F); and
(l) combinations thereof.

6. The polypeptide of any one of claims 1 to 2, wherein the one or more modifications are selected from the group consisting of:
(a) an Arg at position 67 of SEQ ID NO: 1 (L67R), and a Trp at position 68 of SEQ ID NO: 1 (H68W);
(b) a His at position 67 of SEQ ID NO: 1 (L67H), and a Tyr at position 68 of SEQ ID NO: 1 (H68Y); and
(c) a His at position 67 of SEQ ID NO: 1 (L67H), and a Phe at position 68 of SEQ ID NO: 1 (H68F);
(d) a His at position 67 of SEQ ID NO: 1 (L67H), and a Trp at position 68 of SEQ ID NO: 1 (H68W);
(e) an Arg at position 67 of SEQ ID NO: 1 (L67R), and a Tyr at position 68 of SEQ ID NO: 1 (H68Y);
(f) an Arg at position 67 of SEQ ID NO: 1 (L67R), and a Phe at position 68 of SEQ ID NO: 1 (H68F);
(g) a Ser at position 67 of SEQ ID NO: 1 (L67S), and a Phe at position 68 of SEQ ID NO: 1 (H68F);
(h) a Thr at position 67 of SEQ ID NO: 1 (L67T), and a Trp at position 68 of SEQ ID NO: 1 (H68W);
(i) a Ser at position 67 of SEQ ID NO: 1 (L67S), and a Trp at position 68 of SEQ ID NO: 1 (H68W);
(j) a Gln at position 67 of SEQ ID NO: 1 (L67Q), and a Tyr at position 68 of SEQ ID NO: 1 (H68Y);
(k) an Asn at position 67 of SEQ ID NO: 1 (L67N), and a Tyr at position 68 of SEQ ID NO: 1 (H68Y);
(l) an Asn at position 67 of SEQ ID NO: 1 (L67N), and a Trp at position 68 of SEQ ID NO: 1 (H68W); or
(m) a Lys at position 67 of SEQ ID NO: 1 (L67K), and a Tyr at position 68 of SEQ ID NO: 1 (H68Y).

7. The polypeptide of claim 1, wherein the polypeptide comprises the sequence of SEQ ID NO: 2.

8. The polypeptide of claim 1, wherein the polypeptide comprises the sequence of SEQ ID NO: 3.

9. The polypeptide of claim 1, wherein the polypeptide comprises the sequence of SEQ ID NO: 4.

10. The polypeptide of claim 1, wherein the polypeptide comprises the sequence of SEQ ID NO: 5.

11. The polypeptide of claim 1, wherein the polypeptide comprises the sequence of SEQ ID NO: 6.

12. The polypeptide of claim 1, wherein the polypeptide comprises the sequence of SEQ ID NO: 7.

13. The polypeptide of claim 1, wherein the polypeptide comprises the sequence of SEQ ID NO: 8.

14. The polypeptide of claim 1, wherein the polypeptide comprises the sequence of SEQ ID NO: 9.

15. The polypeptide of claim 1, wherein the polypeptide comprises the sequence of SEQ ID NO: 10.

16. The polypeptide of claim 1, wherein the polypeptide comprises the sequence of SEQ ID NO: 11.

17. The polypeptide of claim 1, wherein the polypeptide comprises the sequence of SEQ ID NO: 12.

18. The polypeptide of claim 1, wherein the polypeptide comprises the sequence of SEQ ID NO: 13.

19. The polypeptide of claim 1, wherein the polypeptide comprises the sequence of SEQ ID NO: 14.

20. The polypeptide of claim 1, wherein the polypeptide comprises the sequence of SEQ ID NO: 15.

21. The polypeptide of claim 1, wherein the polypeptide comprises the sequence of SEQ ID NO: 16.

22. The polypeptide of claim 1, wherein the polypeptide comprises the sequence of SEQ ID NO: 17.

23. The polypeptide of claim 1, wherein the polypeptide comprises the sequence of SEQ ID NO: 18.

24. The polypeptide of claim 1, wherein the polypeptide comprises the sequence of SEQ ID NO: 19.

25. The polypeptide of claim 1, wherein the polypeptide comprises the sequence of SEQ ID NO: 20.

26. The polypeptide of claim 1, wherein the polypeptide comprises the sequence of SEQ ID NO: 21.

27. The polypeptide of claim 1, wherein the polypeptide comprises the sequence of SEQ ID NO: 22.

28. The polypeptide of claim 1, wherein the polypeptide comprises the sequence of SEQ ID NO: 23.

29. The polypeptide of claim 1, wherein the polypeptide comprises the sequence of SEQ ID NO: 24.

30. The polypeptide of claim 1, wherein the polypeptide comprises the sequence of SEQ ID NO: 25.

31. The polypeptide of claim 1, wherein the polypeptide comprises the sequence of SEQ ID NO: 26.

32. The polypeptide of claim 1, wherein the polypeptide comprises the sequence of SEQ ID NO: 27.

33. The polypeptide of claim 1, wherein the polypeptide comprises the sequence of SEQ ID NO: 28.

34. The polypeptide of claim 1, wherein the polypeptide comprises the sequence of SEQ ID NO: 29.

35. The polypeptide of claim 1, wherein the polypeptide comprises the sequence of SEQ ID NO: 30.

36. The polypeptide of claim 1, wherein the polypeptide comprises the sequence of SEQ ID NO: 31.

37. The polypeptide of claim 1, wherein the polypeptide comprises the sequence of SEQ ID NO: 32.

38. The polypeptide of claim 1, wherein the polypeptide comprises the sequence of SEQ ID NO: 33.

39. The polypeptide of claim 1, wherein the polypeptide comprises the sequence of SEQ ID NO: 34.

40. The polypeptide of claim 1, wherein the polypeptide comprises the sequence of SEQ ID NO: 35.

41. The polypeptide of claim 1, wherein the polypeptide comprises the sequence of SEQ ID NO: 36.

42. The polypeptide of claim 1, wherein the polypeptide comprises the sequence of SEQ ID NO: 37.

43. The polypeptide of claim 1, wherein the polypeptide comprises the sequence of SEQ ID NO: 38.

44. The polypeptide of claim 1, wherein the polypeptide comprises the sequence of SEQ ID NO: 39.

45. The polypeptide of claim 1, wherein the polypeptide comprises the sequence of SEQ ID NO: 40.

46. The polypeptide of claim 1, wherein the polypeptide comprises the sequence of SEQ ID NO: 41.

47. The polypeptide of claim 1, wherein the polypeptide comprises the sequence of SEQ ID NO: 42.

48. The polypeptide of claim 1, wherein the polypeptide comprises the sequence of SEQ ID NO: 43.

49. The polypeptide of claim 1, wherein the polypeptide comprises the sequence of SEQ ID NO: 44.

50. The polypeptide of claim 1, wherein the polypeptide comprises the sequence of SEQ ID NO: 45.

51. The polypeptide of claim 1, wherein the polypeptide comprises the sequence of SEQ ID NO: 46.

52. The polypeptide of claim 1, wherein the polypeptide comprises the sequence of SEQ ID NO: 47.

53. The polypeptide of claim 1, wherein the polypeptide comprises the sequence of SEQ ID NO: 48.

54. The polypeptide of claim 1, wherein the polypeptide comprises the sequence of SEQ ID NO: 49.

55. The polypeptide of claim 1, wherein the polypeptide comprises the sequence of SEQ ID NO: 50.

56. The polypeptide of claim 1, wherein the polypeptide comprises the sequence of SEQ ID NO: 51.

57. The polypeptide of claim 1, wherein the polypeptide comprises the sequence of SEQ ID NO: 52.

58. The polypeptide of claim 1, wherein the polypeptide comprises the sequence of SEQ ID NO: 53.

59. The polypeptide of claim 1, wherein the polypeptide comprises the sequence of SEQ ID NO: 54.

60. The polypeptide of claim 1, wherein the polypeptide comprises the sequence of SEQ ID NO: 55.

61. The polypeptide of claim 1, wherein the polypeptide comprises the sequence of SEQ ID NO: 56.

62. The polypeptide of claim 1, wherein the polypeptide comprises the sequence of SEQ ID NO: 57.

63. The polypeptide of claim 1, wherein the polypeptide comprises the sequence of SEQ ID NO: 58.

64. The polypeptide of claim 1, wherein the polypeptide comprises the sequence of SEQ ID NO: 59.

65. The polypeptide of claim 1, wherein the polypeptide comprises the sequence of SEQ ID NO: 60.

66. The polypeptide of claim 1, wherein the polypeptide comprises the sequence of SEQ ID NO: 61.

67. The polypeptide of claim 1, wherein the polypeptide comprises the sequence of SEQ ID NO: 62.

68. The polypeptide of claim 1, wherein the polypeptide comprises the sequence of SEQ ID NO: 63.

69. The polypeptide of claim 1, wherein the polypeptide comprises the sequence of SEQ ID NO: 64.

70. The polypeptide of claim 1, wherein the polypeptide comprises the sequence of SEQ ID NO: 65.

71. The polypeptide of claim 1, wherein the polypeptide comprises the sequence of SEQ ID NO: 66.

72. The polypeptide of claim 1, wherein the polypeptide comprises the sequence of SEQ ID NO: 67.

73. The polypeptide of claim 1, wherein the polypeptide comprises the sequence of SEQ ID NO: 68.

74. The polypeptide of claim 1, wherein the polypeptide comprises the sequence of SEQ ID NO: 69.

75. The polypeptide of claim 1, wherein the polypeptide comprises the sequence of SEQ ID NO: 70.

76. The polypeptide of claim 1, wherein the polypeptide comprises the sequence of SEQ ID NO: 71.

77. The polypeptide of claim 1, wherein the polypeptide comprises the sequence of SEQ ID NO: 72.

78. The polypeptide of any one of claims 1 to 6, further comprising one or more additional modifications relative to SEQ ID NO: 1.

79. The polypeptide of claim 30, wherein the one or more additional modifications relative to SEQ ID NO: 1 comprise 1, 2, 3, 4, 5, 6, 7, 8, 9, 10, 11, 12, 13, 14, 15, 16, 17, 18, 19, 20 or more modifications at any amino acid position relative to the amino acid sequence of SEQ ID NO: 1.

80. The polypeptide of claim 30, wherein the one or more additional modifications are selected from the group consisting of:
(a) a Gln at position 2 of SEQ ID NO: 1 (L2Q);
(b) an Ile at position 44 of SEQ ID NO: 1 (A44I);
(c) a Gln at position 49 of SEQ ID NO: 1 (S49Q);
(d) a Gln at position 62 of SEQ ID NO: 1 (L62Q);
(e) a Glu at position 64 of SEQ ID NO: 1 (D64E);
(f) a Thr at position 66 of SEQ ID NO: 1 (K66T);
(g) a Leu at position 69 of SEQ ID NO: 1 (P69L);
(h) a Val at position 70 of SEQ ID NO: 1 (L70V); and
(i) combinations thereof.

81. The polypeptide of any one of claims 1 to 6 or 30 to 32, further comprising one or more modifications at:
(a) position 12 of SEQ ID NO: 1;
(b) position 14 of SEQ ID NO: 1;
(c) position 65 of SEQ ID NO 1;
(d) position 66 of SEQ ID NO: 1;
(e) position 67 of SEQ ID NO: 1; and
(f) combinations thereof.

82. The polypeptide of claim 81, wherein the modification at position 12 of SEQ ID NO: 1 is Tyr (T12Y).

83. The polypeptide of claim 81, wherein the modification at position 12 of SEQ ID NO: 1 is Val (T12V).

84. The polypeptide of any one of claims 81 to 82, wherein the modification at position 14 of SEQ ID NO: 1 is Glu (T14E).

85. The polypeptide of any one of claims 81 to 82, wherein the modification at position 14 of SEQ ID NO: 1 is His (T14H).

86. The polypeptide of any one of claims 81 to 85, wherein the modification at position 65 of SEQ ID NO: 1 is Lys (S65K).

87. The polypeptide of any one of claims 81 to 86, wherein the modification at position 66 of SEQ ID NO: 1 is Gly (K66G).

88. The polypeptide of any one of claims 81 to 87, wherein the modification at position 67 of SEQ ID NO: 1 is His (L67H).

89. The polypeptide of any one of claims 1 to 88, further comprising one or two Gly at the C-terminal end of the polypeptide.

90. The polypeptide of any one of claims 1 to 89, wherein the polypeptide has a binding affinity to the 53BP1 Tudor domain of 0.5 to 500×10⁻⁹ M.

91. A composition comprising the polypeptide of any one of claims 1 to 90 in admixture with a carrier, excipient or diluent.

92. A polynucleotide comprising a nucleic acid sequence encoding the polypeptide of any one of claims 1 to 90.

93. An expression vector comprising the polynucleotide of claim 92.

94. A composition comprising the polypeptide of any one of claims 1 to 90, the composition of claim 91, the polynucleotide of claim 92, or the expression vector of claim 93 and one or more components of a gene editing system.

95. The composition of claim 94, wherein the one or more components of the gene editing system comprise:
(i) a nuclease capable of generating a double-strand break within a gene locus of a cell; and
(ii) a donor polynucleotide.

96. The composition of claim 95, wherein the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

97. The composition of any one of claims 95 to 96, wherein the nuclease comprises a CRISPR nuclease and a single guide RNA (sgRNA) capable of hybridizing to a target sequence within the gene locus, wherein the sgRNA guides the CRISPR nuclease to the target sequence.

98. The composition of claim 97, wherein the CRISPR nuclease is a Cas protein.

99. The composition of claim 98, wherein the Cas protein is Cas9 or a high-fidelity variant thereof.

100. The composition of any one of claims 97 to 99, wherein the sgRNA and the CRISPR nuclease are formed in a ribonucleoprotein (RNP) complex.

101. The composition of any one of claims 97 to 100, wherein the sgRNA comprises one or more chemically modified nucleotides.

102. The composition of claim 101, wherein the modified nucleotide is selected from the group consisting of: a 2′-O-methyl nucleotide, a 2′-O-methyl 3′-phosphorothioate nucleotide, and a 2′-O-methyl 3′-thioPACE nucleotide.

103. The composition of any one of claims 101 to 102, wherein a 5′ end, a 3′ end, or a combination thereof of the modified sgRNA comprises a modified nucleotide.

104. The composition of any one of claims 95 to 103, wherein the donor polynucleotide is comprised in a viral vector, a plasmid, or a single-stranded oligodeoxynucleotide (ssODN).

105. The composition of any one of claims 95 to 104, wherein the donor polynucleotide is comprised in an adeno-associated viral (AAV) vector.

106. The composition of claim 105, wherein the AAV vector is selected from the group consisting of AAV1, AAV2, AAV3, AAV4, AAV3, AAV5, AAV6, AAV7, AAV8, AAV9, AAV10, AAV11, and AAV12.

107. The composition of claim 105, wherein the AAV vector is an AAV6 vector.

108. The composition of any one of claims 94 to 107, further comprising an inhibitor of DNA-dependent protein kinase catalytic subunit (DNA-PKcs).

109. The composition of claim 108, wherein the DNA-PKcs inhibitor is selected from the group consisting of AZD7648, M3814/nedesertib, CC-115 and BAY-8400.

110. The composition of any one of claims 95 to 109, wherein the gene locus is the HBB gene.

111. A host cell comprising the polypeptide of any one of claims 1 to 90 or the composition of claim 91.

112. A host cell comprising the polynucleotide of claim 92 or the expression vector of claim 93.

113. A host cell comprising the composition of any one of claims 94 to 110.

114. The host cell of any one of claims 111 to 113, wherein the host is a mammal.

115. The host cell of claim 114, wherein the mammal is a human.

116. The host cell of any one of claims 11 to 115, wherein the cell is a primary cell.

117. The host cell of claim 116, wherein the primary cell is selected from the group consisting of a primary blood cell and a primary mesenchymal cell.

118. The host cell of claim 116, wherein the primary cell is selected from the group consisting of a primary stem cell, primary progenitor cell, and primary somatic cell.

119. The host cell of claim 118, wherein the primary stem cell is selected from the group consisting of an embryonic stem cell, induced pluripotent stem cell, hematopoietic stem cell, mesenchymal stem cell, neural stem cell, and organ stem cell.

120. The host cell of claim 118, wherein the primary progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell.

121. The host cell of claim 118, wherein the primary somatic cell is selected from the group consisting of a fibroblast, a hepatocyte, a heart cell, a liver cell, a pancreatic cell, a muscle cell, a skin cell, a blood cell, a neural cell, and an immune cell.

122. The host cell of claim 121, wherein the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell.

123. The host cell of claim 116, wherein the primary cell is a CD34+ hematopoietic stem or progenitor cell.

124. The host cell of any one of claims 111 to 123, wherein a gene locus of the cell comprises one or more mutations associated with a disease or encodes an aberrant protein.

125. The host cell of any one of claims 113 to 124, wherein integration of the donor polynucleotide sequence into the host cell is capable of correcting a mutation in the cell that is associated with a disease.

126. The host cell of any one of claims 124 to 125, wherein the disease is selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders.

127. The host cell of claim 126, wherein the hemoglobinopathy is sickle cell disease, α-thalassemia, β-thalassemia, or δ-thalassemia.

128. The host cell of any one of claims 113 to 124, wherein integration of the donor polynucleotide sequence is capable of replacing a mutant allele in the host cell with a wild-type allele.

129. A kit comprising:
the polypeptide of any one of claims 1 to 90, the polynucleotide of claim 92, or the expression vector of claim 93;
one or more components of a gene editing system.

130. The kit of claim 129, wherein the one or more components of a gene editing system comprise:
(i) a nuclease capable of generating a double-strand break within a gene locus of a cell; and
(ii) a donor polynucleotide.

131. The kit of claim 130, wherein the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

132. The kit of any one of claims 130 to 131, wherein the nuclease comprises a CRISPR nuclease and a single guide RNA (sgRNA) capable of hybridizing to a target sequence within the gene locus, wherein the sgRNA guides the CRISPR nuclease to the target sequence.

133. The kit of claim 132, wherein the CRISPR nuclease is a Cas protein.

134. The kit of claim 133, wherein the Cas protein is Cas9 or a high-fidelity variant thereof.

135. The kit of any one of claims 132 to 134, wherein the sgRNA and the CRISPR nuclease are formed in a ribonucleoprotein (RNP) complex.

136. The kit of any one of claims 132 to 135, wherein the sgRNA comprises one or more chemically modified nucleotides.

137. The kit of claim 136, wherein the modified nucleotide is selected from the group consisting of: a 2′-O-methyl nucleotide, a 2′-O-methyl 3′-phosphorothioate nucleotide, and a 2′-O-methyl 3′-thioPACE nucleotide.

138. The kit of any one of claims 136 to 137, wherein a 5′ end, a 3′ end, or a combination thereof of the modified sgRNA comprises a modified nucleotide.

139. The kit of any one of claims 130 to 138, wherein the donor polynucleotide is comprised in a viral vector, a plasmid, or a single-stranded oligodeoxynucleotide (ssODN).

140. The kit of any one of claims 130 to 139, wherein the donor polynucleotide is comprised in an adeno-associated viral (AAV) vector.

141. The kit of claim 140, wherein the AAV vector is selected from the group consisting of AAV1, AAV2, AAV3, AAV4, AAV3, AAV5, AAV6, AAV7, AAV8, AAV9, AAV10, AAV11, and AAV12.

142. The kit of claim 140, wherein the AAV vector is an AAV6 vector.

143. The kit of any one of claims 129 to 142, further comprising an inhibitor of DNA-dependent protein kinase catalytic subunit (DNA-PKcs).

144. The kit of claim 143, wherein the DNA-PKcs inhibitor is selected from the group consisting of AZD7648, M3814/nedesertib, CC-115 and BAY-8400.

145. The kit of any one of claims 130 to 144, wherein the gene locus is the HBB gene.

146. A method of increasing homologous recombination in a cell comprising administering the polypeptide of any one of claims 1-90 to the cell.

147. The method of claim 146, further comprising introducing to the cell one or more components of a gene editing system.

148. The method of claim 147, wherein the one or more components of the gene editing system comprise:
(i) a nuclease capable of generating a double-strand break within a gene locus of a cell; and
(ii) a donor polynucleotide.

149. A method of stably integrating an exogenous polynucleotide sequence into the genome of a cell, the method comprising introducing into the cell:
(a) a nuclease capable of generating a double-strand break within a gene locus of the cell;
(b) a donor polynucleotide; and
(c) the polypeptide of any one of claims 1 to 90;
whereupon generation of a double-strand break within the gene locus by the nuclease, the donor polynucleotide sequence is integrated into the gene locus by homology directed repair (HDR) to yield gene-edited cells.

150. The method of claim 149, wherein the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

151. The method of any one of claims 149 to 150, wherein the nuclease comprises a CRISPR nuclease and a single guide RNA (sgRNA) capable of hybridizing to a target sequence within the gene locus, wherein the sgRNA guides the CRISPR nuclease to the target sequence.

152. The method of claim 151, wherein the CRISPR nuclease is a Cas protein.

153. The method of claim 152, wherein the Cas protein is Cas9 or a high-fidelity variant thereof.

154. The method of any one of claims 151 to 153, wherein the sgRNA and the CRISPR nuclease are formed in a ribonucleoprotein (RNP) complex prior to introducing into the cell.

155. The method of any one of claims 151 to 154, wherein the sgRNA comprises one or more chemically modified nucleotides.

156. The method of claim 155, wherein the modified nucleotide is selected from the group consisting of: a 2′-O-methyl nucleotide, a 2′-O-methyl 3′-phosphorothioate nucleotide, and a 2′-O-methyl 3′-thioPACE nucleotide.

157. The method of claim 155 or 156, wherein a 5′ end, a 3′ end, or a combination thereof of the modified sgRNA comprises a modified nucleotide.

158. The method of any one of claims 149 to 157, wherein the donor polynucleotide is comprised in a viral vector, a plasmid, or a single-stranded oligodeoxynucleotide (ssODN).

159. The method of any one of claims 149 to 158, wherein the donor polynucleotide vector is comprised in an adeno-associated viral (AAV) vector.

160. The method of claim 159, wherein the AAV vector is selected from the group consisting of:
AAV1, AAV2, AAV3, AAV4, AAV3, AAV5, AAV6, AAV7, AAV8, AAV9, AAV10, AAV11
and AAV12.

161. The method of claim 159, wherein the AAV vector is an AAV6 vector.

162. The method of any one of claims 149 to 161, further comprising introducing to the cell an inhibitor of DNA-dependent protein kinase catalytic subunit (DNA-PKcs).

163. The method of claim 162, wherein the DNA-PKcs inhibitor is selected from the group consisting of AZD7648, M3814/nedesertib, CC-115 and BAY-8400.

164. The method of any one of claims 149 to 163, wherein the gene locus is the HBB gene.

165. The method of any one of claims 149 to 164, wherein the host is a mammal.

166. The method of claim 165, wherein the mammal is a human.

167. The method of any one of claims 149 to 166, wherein the cell is a primary cell.

168. The method of claim 167, wherein the primary cell is selected from the group consisting of a primary blood cell and a primary mesenchymal cell.

169. The method of claim 167, wherein the primary cell is selected from the group consisting of a primary stem cell, primary progenitor cell, and primary somatic cell.

170. The method of claim 169, wherein the stem cell selected from the group consisting of an embryonic stem cell, induced pluripotent stem cell, hematopoietic stem cell, mesenchymal stem cell, neural stem cell, and organ stem cell.

171. The method of claim 169, wherein the progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell.

172. The method of claim 169, wherein the somatic cell is selected from the group consisting of a fibroblast, a hepatocyte, a heart cell, a liver cell, a pancreatic cell, a muscle cell, a skin cell, a blood cell, a neural cell, and an immune cell.

173. The method of claim 169, wherein the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell.

174. The method of claim 167, wherein the primary cell is a CD34+ hematopoietic stem or progenitor cell.

175. The method of any one of claims 149 to 174, wherein a gene locus of the cell comprises one or more mutations associated with a disease or encodes an aberrant protein.

176. The method of any one of claims 149 to 174, wherein integration of the donor polynucleotide sequence into the host cell genome corrects a mutation in the cell that is associated with a disease.

177. The method of any one of claims 149 to 174, wherein integration of the donor polynucleotide sequence replaces a mutant allele in the cell with a wild-type allele.

178. The method of claim 175 or 176, wherein the disease is selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders.

179. The method of claim 178, wherein the hemoglobinopathy is sickle cell disease, α-thalassemia, β-thalassemia, or δ-thalassemia.

180. The method of any one of claims 149 to 179, further comprising administering the gene-edited cells to a patient in need thereof.

181. A method of identifying a variant polypeptide of a 53BP1-inhibiting polypeptide having the amino acid sequence of SEQ ID NO:1, wherein the variant polypeptide is capable of mediating improved homology-directed repair (HDR)-mediated integration of a donor polynucleotide, the method comprising the steps of:
a. contacting a CD34+ hematopoietic stem and progenitor cell (HSPC) population with:
i. a control polypeptide comprising the amino acid sequence of SEQ ID NO: 1;
ii. one or more variant polypeptides, wherein each variant polypeptide comprises an amino acid sequence of SEQ ID NO:1 having one or more modifications relative to SEQ ID NO: 1, wherein the one or more modifications comprise a modification at position 67 of SEQ ID NO: 1, a modification at position 68 of SEQ ID NO: 1, or a combination thereof;
iii. a nuclease capable of generating a double-strand break within a gene locus of the HSPCs; and
iv. a donor polynucleotide comprising a reporter gene;
b. isolating:
i. a first subpopulation of contacted HSPCs that expresses at least a threshold amount of the reporter gene; and
ii. a second subpopulation of contacted HSPCs that does not express the threshold amount of the reporter gene; and
c. measuring the relative abundance of control and variant polypeptides in both the first and second HSPC subpopulations, respectively.

182. The method of claim 181, wherein a variant polypeptide is identified as capable of mediating improved homology-directed repair (HDR)-mediated integration relative to the control polypeptide if: (i) the variant polypeptide is more abundant than the control polypeptide in the first subpopulation; and/or (ii) the variant:control polypeptide ratio of the first subpopulation is higher than the variant:control polypeptide ratio of the second subpopulation.

183. The method of claim 181 or 182, wherein each of the one or more variant polypeptides further comprises a modification at position 12 of SEQ ID NO:1, a modification at position 14 of SEQ ID NO:1, or a combination thereof.

184. The method of any one of claims 181 to 183, wherein each of the one or more variant polypeptides further comprises a modification at position 65 of SEQ ID NO:1, a modification at position 66 of SEQ ID NO: 1, or a combination thereof.

185. The method of any one of claims 183 to 184, wherein contacting of the first and second HSPC populations with the control and variant polypeptides, respectively, comprises contacting the first and second HSPC populations with lentiviral vectors encoding the control and variant polypeptides, respectively.

186. The method of any one of claim 185, wherein measuring the relative abundance of control and variant polypeptides comprises sequencing of the lentiviral vectors encoding the control and variant polypeptides.

187. The method of any one of claims 181 to 186, wherein each modification of the variant polypeptide comprises a single amino acid substitution.

188. The method of claim 185, wherein each lentiviral vector encoding a variant polypeptide comprises a polynucleotide comprising an NNK codon encoding a modification of the variant polypeptide.

189. The method of any one of claims 181 to 188, wherein the donor polynucleotide comprises non-overlapping 5′ and 3′ homology arms, wherein each homology arm is homologous to a portion of the gene locus, whereupon generation of the double-strand break within the gene locus by the nuclease, the donor polynucleotide sequence is integrated into the gene locus by homology directed repair (HDR).

190. The method of any one of claims 181 to 189, wherein the nuclease comprises a CRISPR nuclease and a single guide RNA (sgRNA) capable of hybridizing to a target sequence within the gene locus, wherein the sgRNA guides the CRISPR nuclease to the target sequence.

191. The method of claim 190, wherein the sgRNA and the CRISPR nuclease are formed in a ribonucleoprotein (RNP) complex.

192. The method of any one of claims 181 to 191, wherein the donor polynucleotide is comprised in a viral vector, a plasmid, or a single-stranded oligodeoxynucleotide (ssODN).

193. The method of claim 192, wherein the donor polynucleotide is comprised in an adeno-associated viral (AAV) vector.

194. The method of any one of claims 181 to 193, further comprising isolating a variant polypeptide having improved HDR activity from the second HSPC population.
